SEQUENCE LISTING (7 sequences)

SEQ ID NO: 1 - RNA, 75 nt, Abelia Sanguinea
  source 1..75: mol_type = mRNA
atcaattcggtattcgtcaaagtttcatttattcaatgaccagaactaaacggggatatacagctcggagacgta

SEQ ID NO: 2 - RNA, 35 nt, Abelia Sanguinea
  source 1..35: mol_type = rRNA
gccgtactgcaaggtgcggctggatcacctccttt

SEQ ID NO: 3 - RNA, 75 nt, Abelia Sanguinea
  source 1..75: mol_type = mRNA
tatgaaaaatgtaaccgattccttcgtttctttgggccactggccatccgccgggagtttcgggtttaataccga

SEQ ID NO: 4 - RNA, 77 nt, synthetic construct
  source 1..77: mol_type = other RNA
tgtggttttgatccacgtttcttggttggtctgactggatcacctatttagtccttatgtctttgtttttgtcgtta

SEQ ID NO: 5 - RNA, 135 nt, synthetic construct
  source 1..135: mol_type = other RNA
tgatgcagcctagaannnnnnaggactttatattggatcagtttagaagtaannnnnnnnnnnnnnnnnnnnnnnnnnnnnnnnnnnntagattnnntgcnnnnnnnnnttgggccttgtatggtaaccctttgc

SEQ ID NO: 6 - RNA, 86 nt, synthetic construct
  source 1..86: mol_type = other RNA
nnnagtaaagaagtttaacgagaggtaaagattcaagtatcggaagcgccatcgaagtagctacaatggatgatttattttccgga

SEQ ID NO: 7 - RNA, 94 nt, synthetic construct
  source 1..94: mol_type = other RNA
tatttctttatcacctaaggtagcaaagataccnnnnnnaatgnnnnnnnnnaagaatttgccactccagaagagatnatgtggctcggaaatg

(19) United States
(12) Patent Application Publication
(10) Pub. No.: US 2025/0316326 A1
(43) Pub. Date: Oct. 9, 2025

(54) OPTIMIZATION OF TRANSLATION IN CHLOROPLASTS

(71) Applicant: RAMOT AT TEL-AVIV UNIVERSITY LTD., Tel Aviv (IL)

(72) Inventors: Tamir TULLER, Tel Aviv (IL); Stav CARMEL EZRA, Tel Aviv (IL)

(21) Appl. No.: 18/865,757

(22) PCT Filed: May 17, 2023
(86) PCT No.: PCT/IL2023/050510

§ 371 Date: Nov. 14, 2024

Related U.S. Application Data
(60) Provisional application No. 63/342,807, filed on May 17, 2022.

(51) Int. Cl. G16B 15/10; G16B 25/10; G16B 30/10

(52) U.S. Cl. CPC G16B 15/10; G16B 25/10; G16B 30/10

(57) ABSTRACT

Methods of predicting translation initiation efficiency in chloroplasts comprising calculating free folding energy of a region in a 5′ UTR, of a region in a16S rRNA, and of 5′ UTR region hybridized to the 16S rRNA region are provided. Methods of determining a region regulating translation of an mRNA in chloroplasts as well as methods of modulating translation of a target mRNA are also provided. mRNAs produced by methods of the invention and DNAs encoding those mRNAs are also provided.

### CROSS REFERENCE TO RELATED APPLICATIONS

This application claims the benefit of priority of U.S. Provisional Patent Application No. 63/342,807 filed on May 17, 2022, the contents of which are all incorporated herein by reference in their entirety.

### REFERENCE TO AN ELECTRONIC SEQUENCE LISTING

The contents of the electronic sequence listing (RMT-P-019-PCT SQL.xml; Size: 7,414 bytes; and Date of Creation: May 17, 2023) is herein incorporated by reference in its entirety.

### FIELD OF INVENTION

The present invention is in the field of protein expression in chloroplasts.

### BACKGROUND OF THE INVENTION

Chloroplasts are intracellular organelles in plants that are responsible for photosynthesis and carbon fixation; they have been generated as a result of an endosymbiosis process in which a eubacterium was engulfed by a common eukaryotic ancestor. Chloroplasts contain their own genetic system with a circular double-stranded DNA molecule, some endosymbiont genes were lost, while others were transferred into the host genome as a result of coevolution between the host and the endosymbiont, which led to a significantly smaller genome which its size is usually 120-160 kb in most chloroplasts, containing ˜120 genes on average, most of which are essential for chloroplast viability because they encode essential components of the photosynthesis machinery. The size, structure, and genetic content of chloroplast genomes of land plants appear to be relatively conserved. It is reported that ˜80% of the genes present in chloroplast genomes of land plants and the most ancient algae, which is a green algae species (Mesostigma viride), are shared; this indicates that both gene content and gene order are generally conserved in chloroplast genomes throughout evolution. The majority of chloroplasts translation studies have been carried out on land plants (‘green’ phylogenetic lineage, e.g., tobacco, maize, spinach, and barley), as well as on chlorophyte green algae (e.g., Chlamydomonas reinhardtii), and on Euglena gracilis which is not a chlorophyte but a member of the euglenoid algae. The mechanism of chloroplasts translation developed in one organism may not be conserved throughout all chloroplasts' lineages due to evolution and diversification over the past 1-2 billion years, this is why there is less information regarding chloroplast translation of other algal lineages, and it is poorly understood.

The chloroplast's translational machinery is most closely related to that of eubacteria, but there are some similarities with the nuclear-cytosolic system of eukaryotes. A highly similar composition of the translation machinery between chloroplasts and bacteria indicates the bacterial origin of the chloroplast gene expression mechanism. Chloroplast translation is performed by prokaryotic-type 70S ribosomes, which consist of a small 30S and a large 50S subunits composed of orthologs of Escherichia coli's (E. coli) reference ribosome rRNAs and proteins. Over the years, the similarity between the translation initiation of prokaryotes and chloroplasts was questioned, and the search for differences between the two mechanisms' features has attracted attention. In all systems studied to date, the translation initiation in chloroplasts starts when a complex consisting of the 30S subunit of the ribosome and the initiator tRNA (N formylmethionine), called the preinitiation complex, binds the initiation site in the mRNA. Three protein initiation factors (IF), IF1, IF2, and IF3, which were found as an ortholog of the bacterial IFs, control and accurate initiation process steps.

The translation initiation model in prokaryotes can be described by the Shine-Dalgarno (SD) mechanism. According to this model, the 30S subunit of the ribosome binds the mRNA through base-pairing between the SD sequence (another name for the RBS) of the mRNA, which is located upstream to the start codon, and the anti-SD (aSD), a conserved sequence found at the 3′-edge of the 16S rRNA of the small subunit of the ribosome. The role of the SD motif in chloroplasts has raised questions and was a source of great research. According to research done in this field to answer these questions, it was found out that there does not seem to be any obvious signal indicating conserved sequences in the SD position at chloroplasts' mRNAs. However, the 16S aSD sequence is highly conserved across chloroplasts. It was discovered that 38% (30 out of 79) of tobacco chloroplasts genes contain no SD-like sequences within 20 nucleotides (nt) upstream from the start codon, and 14% of the genes have the SD-like sequence but not in the expected positions of −18-−16 upstream the start codon, therefore there are only 48% genes with SD-like sequence in the expected positions at the 5′ UTRs of their mRNAs. Several studies tried to investigate the role of the SD-aSD interaction in Chlamydomonas reinhardtii, Euglena, and tobacco chloroplasts' genes by site-directed replacement mutations or deletion of the SD-like sequences and revealed that some genes require SD-like sequence for translation while others do not. More specifically, on some genes, the altered SD positions had little or no effect on their translation in vivo, genes such as petD, atpB, atpE, rps4, rps7 (Chlamydomonas reinhardtii), and rbcL (Euglena), furthermore genes rpl2 and rpls16 (tobacco) do not even have such sequences. On the other hand, it did not affect the translation of genes such as psbA, psbD, psbC (Chlamydomonas reinhardtii), atpH (Euglena), rps14 (tobacco). Thus, this SD-like element has a positive role in their translation initiation. It has been suggested that the requirement for SD-like sequences may be more important for the translation of highly expressed mRNAs in Chlamydomonas reinhardtii. The lack of an absolute requirement for the SD-like sequences of several mRNAs indicates that other cis-acting elements recruit ribosomes to the start codon position.

Previous research has also shown that the chloroplasts' ribosomal RNA and ribosomal proteins differ from those of E. coli; the ratio between ribosomal proteins and rRNA significantly shifted during evolution, favoring ribosomal proteins, which led to modifications in the rRNA domains. As a result, ribosomal proteins interact differently with rRNAs or other ribosomal proteins and perform structural changes that compensate for altered rRNA domains. These ribosomal changes may result in new contact sites with the mRNA molecule and therefore are hypothesized to affect translational regulation. Additionally, it was discovered that point mutations are leading to changes in the local structure of ribosomal RNA in chloroplasts; these mutations create significantly folded structures in the positions of the a-SD, which reduce the probability of ribosome binding to the mRNA.

It was discovered that there are protein factors (trans-acting factors) that mediate translation initiation by interacting with the mRNA sequence or secondary structures at the 5′ UTR located in cis, typically upstream of the reading frame in the mRNA. These factors are gene-specific and were discovered for specific chloroplasts' genomes. Some specific cis-elements controlling the translation of individual genes in a particular chloroplast genome were studied and discovered; however, the molecular function of RNA cis-elements and proteinaceous trans-factors in the regulation of chloroplast translation, in general, is mostly unknown. In addition to primary sequence elements, features of mRNA 2D or 3D structure (or lack of structure) can represent cis-elements that influence the translation process; there are some chloroplasts' genes, for example, that their mRNA molecule conducts a secondary structure such that it reveals the ribosome binding site or the start codon that triggers the translation initiation.

In summary, today there are various pieces of evidence regarding the nature of translation initiation in chloroplasts; however, there is no unified model that can predict translation initiation in chloroplasts. A unified system that allows for genetic engineering of improved chloroplasts is greatly needed.

### SUMMARY OF THE INVENTION

The present invention provides methods of predicting translation initiation efficiency in chloroplasts comprising calculating free folding energy of a region in a 5′ UTR, of a region in a16S rRNA, and of the 5′ UTR region hybridized to the 16S rRNA region are provided. Methods of determining a region regulating translation of an mRNA in chloroplasts as well as methods of modulating translation of a target mRNA are also provided. mRNAs produced by methods of the invention and DNAs encoding those mRNAs are also provided.

According to a first aspect, there is provided a method of predicting translation initiation efficiency of an mRNA comprising a 5′ untranslated region (UTR) and a coding region in a chloroplast, the method comprising:
a. calculating a free folding energy for a region of the 5′ UTR to produce a target free folding energy;
b. calculating a free folding energy for a region of a16S rRNA of the chloroplast to produce an rRNA free folding energy; and
c. calculating a free folding energy for the 5′ UTR region hybridized to the 16S rRNA region to produce a combined free folding energy, wherein a lower combined free folding energy as compared to a sum of the target free folding energy and the rRNA free folding energy indicates translation initiation and the magnitude by which the combined free folding energy is lower is proportionate to translation initiation efficiency;
thereby predicting translation initiation efficiency.

According to some embodiments, the method comprises performing steps a-c for a plurality of regions within the 5′ UTR and selecting the region with the lowest combined free folding energy.

According to some embodiments, the method is for predicting protein expression from the mRNA in the chloroplast, wherein the predicted translation initiation efficiency is proportional to the predicted protein expression.

According to some embodiments, the method further comprises confirming the prediction by comparing the predicted translation initiation efficiency to received expression levels of the mRNA in the chloroplast.

According to some embodiments, the method is a method of predicting expression level of a protein encoded by the mRNA in the chloroplast and wherein the greater the difference between the combined free folding energy and the sum of the target free folding energy and the rRNA free folding energy the greater the expression level of the protein in the chloroplast.

According to some embodiments, the method further comprises receiving a measure of protein expression levels in the chloroplast of a protein translated from the mRNA and correlating predicted expression levels to the measure.

According to some embodiments, the received expression levels are approximated by the codon adaptation index (CAI) in the mRNA.

According to some embodiments, the method further comprises optimizing the correlation, wherein the optimizing comprises providing a plurality of mRNAs of proteins expressed in the chloroplast, selecting a subgroup of the plurality as a training set and a subgroup of the plurality as a test set, selecting a parameter that optimizes correlation between the predicted expression levels in the training set to the measure of protein expression and validating the parameter in the test set.

According to some embodiments, the parameter is selected from 5′ UTR region length, 5′ UTR region start position, 16S rRNA region length and a correction factor applied to the sum of the target free folding energy and the rRNA free folding energy.

According to another aspect, there is provided a method of determining a region regulating translation in an mRNA comprising a 5′ UTR, a coding region and a 3′ UTR in a chloroplast, the method comprising:
a. providing a database of sequences of the mRNA in chloroplast in a plurality of species;
b. aligning the sequences based on sequence similarity to produce a multisequence alignment (MSA);
c. calculating a free folding energy for a region of the mRNA and for each sequence aligned with the region in the MSA to produce a target free folding energy;
d. selecting a region in which the target free folding energy is lower than a free folding energy in a null model;
e. performing a method of the invention to predict translation initiation efficiency for the selected region of step (d); and
f. selecting a region as a region initiating translation if the combined free folding energy is lower than a sum of the target free folding energy and the rRNA free folding energy and a region as a region terminating translation is the combined free folding energy is higher than the sum;
thereby determining a region regulating translation.

According to some embodiments, the method is for determining secondary mRNA structures that initiate or terminate translation, wherein the structure is the mRNA structure of the selected region.

According to some embodiments, the database comprises sequences from at least 10 different species.

According to some embodiments, the region is a window of 25-50 nucleotides.

According to some embodiments, the region is within the 5′ untranslated region (UTR) and the regulating translation is initiating translation or the region is within the 3′ UTR and the regulating translation is terminating translation.

According to some embodiments, the method comprises evaluating all possible regions within the mRNA.

According to some embodiments, the method comprises combining any adjacent regions that all initiated translation or terminate translation to produce a complete initiating region, a complete terminating region or both.

According to some embodiments, the lower is lower by more than a predetermined threshold, the higher is higher by more than a predetermined threshold or both.

According to some embodiments, the calculating free folding energy comprises calculating relative free folding energy and comprises calculating free folding energy for a null model of the sequence of the region or aligned sequence, wherein the relative free folding energy is the difference between the free folding energy of the region or aligned sequence and the null model.

According to some embodiments, the region does not comprise a Shine Dalgarno sequence or comprises a Shine Dalgarno sequence at a location that is not between position −1 and −16 with respect to the translational start site of the mRNA.

According to some embodiments, the selecting further comprises selecting a region or combined region with relative free folding energy that is below a predetermined threshold, thus selecting a region with a conserved structure.

According to some embodiments, the conserved structure is conserved in all species of the plurality of species.

According to some embodiments, the selecting comprises selecting a region or combined region comprising a relative free folding energy that is significantly lower than the relative free folding energy of both the adjacent upstream and downstream regions.

According to another aspect, there is provided a method of modulating translation of a target mRNA in a chloroplast, the method comprising determining a region regulating translation in the chloroplast by a method of the invention; and
i. generating the determined region in the target mRNA which is not an mRNA that naturally comprises the determined region; or
ii. abolishing the determined region in the target mRNA which is an mRNA that naturally comprises the determined region;
thereby modulating translation of a target mRNA.

According to another aspect, there is provided a method of modulating translation of a target mRNA in a chloroplast, the method comprising generating in the target mRNA a region folding into a secondary structure selected from those provided in Table 3 or abolishing in the target mRNA a secondary structure selected from those provided in Table 3; thereby modulating translation of a target mRNA.

According to some embodiments, the generating comprises:
a. insertion of the determined region or a region folding into the secondary structure into the mRNA or mutation of a region of the target mRNA to produce the determined region; or
b. insertion of the determined region or a region folding into the secondary structure into a DNA encoding the target mRNA or mutation of a region of a DNA encoding the target mRNA to produce the determined region.

According to some embodiments, the abolishing comprises deleting the determined region or region folding into the secondary structure or mutating the determined region or region folding into the secondary structure.

According to some embodiments, the mutating changes the local folding energy of the determined region in the mRNA by at least a predetermined threshold.

According to some embodiments, the generated is at a location in the target mRNA that corresponds to the location of the determined region or region folding into the secondary structure in the mRNA from which it was determined; optionally wherein the determined region or region folding into the secondary structure is located in its original mRNA in a 5′ UTR and is generated in a 5′ UTR of the target mRNA or is located in its original mRNA in a 3′ UTR and is generated in a 3′ UTR of the target mRNA.

According to another aspect, there is provided an mRNA molecule produced by a method of the invention.

According to another aspect, there is provided a DNA molecule comprising an open reading frame encoding an mRNA molecule of the invention.

According to some embodiments, the DNA molecule further comprises at least one regulatory element operatively linked to the open reading frame, optionally wherein the at least one regulatory element induces transcription in the chloroplast.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: The general flow diagram of the study.

FIG. 2: Illustrations of the local self-folding and co-folding of the mRNA and rRNA. 2.1) The local self-folding of the mRNA. 2.2) The local self-folding of the 16S rRNA from the 3′ edge. 2.3) The local interaction between the mRNA and the 16S rRNA, with the four typical properties of the local structures and hybridization: 1. mRNA window size—the length of the mRNA portion that interacts with the 16S sequence of the ribosome, 2. The start position at the mRNA—the position, relative to the start codon, of the first nucleotide of the mRNA window sequence, 3. 16S rRNA length—the length of the 16S portion from its 3′end that interacts with the mRNA, 4. The model's constant that determines the subtraction of the self-foldings from the co-folding energy and corrects second-order aspects of translation regulation.

FIGS. 3A-F: Examples of the local self-folding and co-folding of the mRNA (length of 75 nt, one nt upstream of the start codon) and 16S rRNA (length of 35 nt from the 3′ end), of Abelia Sanguinea's chloroplast, and the flow diagram of the energy-based gene expression prediction. (3A) mRNA folding of rpl20 gene (ribosomal protein L20). (3B) 16S rRNA folding. (3C) Co-folding of 3A and 3B. (3D) mRNA folding of atpF gene (ATP synthase CFO subunit I). (3E) Co-folding of 3D and 3B. (3F) Flow diagram of the energy-based gene expression prediction.

FIGS. 4A-D: Optimal Spearman correlations of ETIP model. (4A) Optimal Spearman correlations of real genomes, and for every X (number of parameters that limits the degree of freedom). (4B) Distances between optimal correlations of real and random genomes for every X. (4C) Optimal correlation between ETIP energies (x-axis) and Z-scored CAI scores (y-axis) when the dots correspond to the energy and Z-scored CAI of all the genes in the test set. (4D) All genomes optimal correlations, Pv of the genome with the median correlation.

FIGS. 5A-D: All the groups' optimal energy parameters of ETIP model. (5A) Window length of mRNA. (5B) Window length of 16S. (5C) Position upon 5′ UTR. (5D) ETIP Constant.

FIGS. 6A-C: Histograms of Chlamydomonas reinhardti's genes divided into typical and non-typical groups showing (6A) PA, (6B) scores of aSD-SD interaction, and (6C) positions of aSD-SD interaction.

FIGS. 7A-D: Spearman Correlations of predicted PA values of Chlamydomonas reinhardtii (7A) predicted PA by CAI scores, and (7B) predicted PA based on CAI scores and ETIP energy values. (7C) Predicted ribo-seq values by CAI scores. (7D) Predicted ribo-seq values based on CAI scores and ETIP energy values.

FIGS. 8A-F: P values and Z-scores related to a selection for/against strong energy (i.e., stronger/weaker folding in comparison to the null model) according to positions along the mRNA. (8A-B) Heat maps of the significant P values for selection for (red)/against (green) strong energy in different positions along the mRNA with respect to the (8A) start codon and (8B) the stop codon. (8C-F) Number of groups in percent of orthologous groups (y-axis) that have significant Z-score values in different positions (x-axis) along the mRNA of the real groups (blue) compared to the null model (orange). (8C) Significant positive Z-score values; alignment to the 5′ UTR, (8D) Significant positive Z-score values; alignment to the 3′ UTR. (8E) Significant negative Z-score; alignment to the 5′ UTR. (8F) Significant negative Z-score; alignment to the 3′ UTR.

FIGS. 9A-J: The general statistics related to the detected conserved structures. (9A) Number of structures at the 5′UTR. (9B) Number of structures at the 3′UTR. (9C) Structures' lengths at the 5′UTR. (9D) Structures' lengths at 3′UTR. (9E) Structures' frequencies at the 5′UTR. (9F) Structures' frequencies at the 3′UTR. (9G) Structures' diversities at the 5′UTR. (9H) Structures' diversities at the 3′UTR. (9I) Structures' energies at the 5′UTR. (9J) Structures' energies at the 3′UTR.

FIGS. 10A-D: Examples of consensus secondary structures. Consensus secondary structure in the (10A) 5′ UTR of psbC; its length is 75 nt and it is located 806 nt upstream the start codon (10B) 5′ UTR of infA; its length is 288 nt and it is located, 244 nt upstream the start codon, (10C) 3′ UTR of atpB; its length is 85 nt and it is located, 82 nt upstream the stop codon and (10D) 5′ UTR of rpl20; its length is of 94 nt and it is located 880 nt upstream the start codon.

FIGS. 11A-B: Number of groups in precents that contribute a nucleotide in a position upon the mRNA of the orthologue group, the x axis refers to the position upon the mRNA and the y axis is the groups precent (11A) at the 5′UTR, when zero refers to the start codon, and (11B) at the 3′UTR, when zero refers to the stop codon.

FIGS. 12A-F: Optimal Spearman correlations of the real model, and for every X (number of parameters that limits the degree of freedom) and for every energy type for (12A) Fold, (12B) Cofold, and (12C) ETIP. Distances between the optimal correlations of the real and the null models for every X and, for every energy model for (12D) Fold, (12E) Cofold, and (12F) ETIP.

FIGS. 13A-C: Dot plot of optimal energies and Z-scored CAI for every energy type for (13A) Fold, (13B) Co-fold, and (13C) ETIP.

FIGS. 14A-I: All the groups' optimal energy parameters for every model. (14A) mRNA window length fold; (14B) Position upon 5′UTR fold; (14C) mRNA window length cofold; (14D) 16S window length cofold; (14E) Position upon 5′UTR cofold; (14F) mRNA length ETIP; (14G) 16S window length ETIP; (14H) position upon 5′UTR ETIP; and (14I) Constant of ETIP.

FIGS. 15A-C: Histogram of all the genomes' spearman correlations of Z-scored CAI index for every model with the Pv of the genome with the median correlation for (15A) Fold; (15B) Cofold; and (15C) ETIP.

### DETAILED DESCRIPTION OF THE INVENTION

The present invention, in some embodiments, provides methods of determining a region regulating translation of an mRNA in chloroplasts as well as methods of modulating translation of a target mRNA are also provided. mRNAs produced by methods of the invention and DNAs encoding those mRNAs are also provided.

In this study we conducted a predictive energy-based model of translation initiation (ETIP) in chloroplasts that consider the local folding and co-folding energy of the rRNA and the mRNA. A model which combines the ETIP with measures of codon usage is expected to yield a correlation up to 0.71 with protein levels and 0.66 with ribosomal profiling measures. This model is used to engineer genes in the chloroplast with desired expression levels.

We were surprisingly able to find the local energy parameters related to our model that influence the translation regulation for every ortholog group and demonstrated that different gene families in chloroplasts use different parameters and thus probably have different translation mechanism. Our model predicts that in most of the genes in the chloroplasts, translation initiation does not rely only on aSD-SD interaction; and we provide details related to the alternative translation initiation models.

We observed novel patterns of selection for strong mRNA folding at the ends of the transcripts that may be related to unique chloroplast regulatory aspects. In addition, we created a database of 166 predicted functional mRNA structures that are specific to different orthologous groups in chloroplasts that can be also used for modeling and engineering gene expressions in chloroplasts.

By a first aspect, there is provided a method of predicting translation initiation of an mRNA, the method comprising:
a. calculating a free folding energy for a region of the mRNA to produce a target free folding energy;
b. calculating a free folding energy for a region of a 16S ribosomal RNA (rRNA) to produce an rRNA free folding energy; and
c. calculating a free folding energy for the mRNA region hybridized to the 16S rRNA region to produce a combined free folding energy;
thereby predicting translation initiation.

By another aspect, there is provided a method of determining a region regulating translation in an mRNA, the method comprising:
a. providing a database of sequences of the mRNA in a plurality of species;
b. aligning the sequences based on sequence similarity to produce a multisequence alignment (MSA);
c. calculating a free folding energy for a region of the mRNA in the MSA to produce a target free folding energy;
d. selecting a region in which the target free folding energy is lower than a free folding energy in a null model;
c. performing a method of the invention to predict translation initiation efficiency for the selected region of step (d); and
thereby determining a region regulating translation.

In some embodiments, the method is an in vitro method. In some embodiments, the method is a computerized method. In some embodiments, the method is a diagnostic method. In some embodiments, the method is a method of predicting the site of translation initiation. In some embodiments, the method is a method of predicting translation initiation efficiency.

In some embodiments, the region is within the mRNA. In some embodiments, the mRNA comprises a 5′ untranslated region (UTR). In some embodiments, the mRNA comprises a 3′ UTR. In some embodiments, the mRNA comprises an open reading frame. In some embodiments, the mRNA comprises a coding region. In some embodiments, the mRNA is in a cell. In some embodiments, the mRNA is in an organelle of a cell. In some embodiments, translation is translation in a cell. In some embodiments, translation is translation in an organelle. In some embodiments, translation is in vitro translation. In some embodiments, the predicting is predicting translation initiation in a cell. In some embodiments, the predicting is predicting translation initiation in an organelle. In some embodiments, translation initiation is the site of translation initiation. In some embodiments, translation initiation is translation initiation efficiency. In some embodiments, the cell is a eukaryotic cell. In some embodiments, the cell is a plant cell. In some embodiments, the organelle is an organelle with its own rRNA. In some embodiments, the rRNA is a 16S rRNA. In some embodiments, the organelle is a chloroplast. In some embodiments, the organelle is prokaryotic. In some embodiments, the organelle is a mix of prokaryotic and eukaryotic. In some embodiments, the organelle comprises a membrane. In some embodiments, the membrane is a double membrane.

Chloroplasts are plant cells that convert light energy into stable chemical energy via photosynthesis. Chloroplasts have a prokaryotic origin as at some point the eukaryotic plant cell incorporated a prokaryotic chloroplast cell into it. Chloroplast therefore are a unique mix of prokaryotic and eukaryotic and thus basic functions (e.g., translation and transcription) in this organelle is different than other cells/organelles.

In some embodiments, the region is within the mRNA. In some embodiments, the region is a region in the 5′ UTR. In some embodiments, the region is a region in the 5′ UTR of the mRNA. In some embodiments, the region comprises a Shine-Dalgarno sequence. In some embodiments, the region does not comprise a Shine Dalgarno sequence. In some embodiments, the region comprises a Shine-Dalgarno sequence but at a location that is not the canonical Shine-Dalgarno location. In some embodiments, the canonical Shine-Dalgarno location is between positions −18 and −1. In some embodiments, the canonical Shine-Dalgarno location is between positions −16 and −1. In some embodiments, the canonical Shine-Dalgarno location is between positions −12 and −1. In some embodiments, the canonical Shine-Dalgarno location is between positions −9 and −1. In some embodiments, the canonical Shine-Dalgarno location is between positions −8 and −1. In some embodiments, the region does not comprise positions −50 to −1, −40 to −1, −30 to −1, −25 to −1, −20 to −1, −18 to −1, −16 to −1, −12 to −1, −9 to −1 or −8 to −1 within the 5′ UTR. Each possibility represents a separate embodiment of the invention. In some embodiments, the region does not comprise positions −16 to −1 within the 5′ UTR of the mRNA. In some embodiments, the region does not comprise positions −50 to −1 within the 5′ UTR of the mRNA. In some embodiments, the region does not comprise positions −25 to −1 within the 5′ UTR of the mRNA. In some embodiments, the region does not comprise positions −18 to −1 within the 5′ UTR of the mRNA. In some embodiments, the region does not comprise positions −16 to −1 within the 5′ UTR of the mRNA. In some embodiments, the region does not comprise positions −8 to −1 within the 5′ UTR of the mRNA. In some embodiments, the numbering of positions is with respect to the translational start site of the mRNA. In some embodiments, the region is a window of 5-200, 5-150, 5-100, 5-95, 5-90, 5-85, 5-80, 5-75, 5-70, 5-65, 5-60, 5-55, 5-50, 5-45, 5-40, 5-35, 5-30, 10-200, 10-150, 10-100, 10-95, 10-90, 10-85, 10-80, 10-75, 10-70, 10-65, 10-60, 10-55, 10-50, 10-45, 10-40, 10-35, 10-30, 15-200, 15-150, 15-100, 15-95, 15-90, 15-85, 15-80, 15-75, 15-70, 15-65, 15-60, 15-55, 15-50, 15-45, 15-40, 15-35, 15-30, 20-200, 20-150, 20-100, 20-95, 20-90, 20-85, 20-80, 20-75, 20-70, 20-65, 20-60, 20-55, 20-50, 20-45, 20-40, 20-35, 20-30, 25-200, 25-150, 25-100, 25-95, 25-90, 25-85, 25-80, 25-75, 25-70, 25-65, 25-60, 25-55, 25-50, 25-45, 25-40, 25-35, 25-30, 30-200, 30-150, 30-100, 30-95, 30-90, 30-85, 30-80, 30-75, 30-70, 30-65, 30-60, 30-55, 30-50, 30-45, 30-40, 30-35, 35-200, 35-150, 35-100, 35-95, 35-90, 35-85, 35-80, 35-75, 35-70, 35-65, 35-60, 35-55, 35-50, 35-45, or 35-40 nucleotides. Each possibility represents a separate embodiment of the invention. In some embodiments, the region is a window of 25-50 nucleotides. In some embodiments, the region is a window of 25-90 nucleotides. In some embodiments, the region is a window of 85 nucleotides. In some embodiments, the region is a window of 35 nucleotides.

In some embodiments, the region is a region in the rRNA. In some embodiments, the region of the 16S rRNA is a region in the 3′ terminus of the 16S rRNA. In some embodiments, the region of the 16S rRNA comprises an anti-Shine Dalgarno sequence. In some embodiments, the region of the 16S rRNA does not comprise an anti-Shine Dalgarno sequence. In some embodiments, the region is a window of 5-200, 5-150, 5-100, 5-95, 5-90, 5-85, 5-80, 5-75, 5-70, 5-65, 5-60, 5-55, 5-50, 5-45, 5-40, 5-35, 5-30, 10-200, 10-150, 10-100, 10-95, 10-90, 10-85, 10-80, 10-75, 10-70, 10-65, 10-60, 10-55, 10-50, 10-45, 10-40, 10-35, 10-30, 15-200, 15-150, 15-100, 15-95, 15-90, 15-85, 15-80, 15-75, 15-70, 15-65, 15-60, 15-55, 15-50, 15-45, 15-40, 15-35, 15-30, 20-200, 20-150, 20-100, 20-95, 20-90, 20-85, 20-80, 20-75, 20-70, 20-65, 20-60, 20-55, 20-50, 20-45, 20-40, 20-35, 20-30, 25-200, 25-150, 25-100, 25-95, 25-90, 25-85, 25-80, 25-75, 25-70, 25-65, 25-60, 25-55, 25-50, 25-45, 25-40, 25-35, 25-30, 30-200, 30-150, 30-100, 30-95, 30-90, 30-85, 30-80, 30-75, 30-70, 30-65, 30-60, 30-55, 30-50, 30-45, 30-40, 30-35, 35-200, 35-150, 35-100, 35-95, 35-90, 35-85, 35-80, 35-75, 35-70, 35-65, 35-60, 35-55, 35-50, 35-45, or 35-40 nucleotides. Each possibility represents a separate embodiment of the invention. In some embodiments, the region is a window of 25-50 nucleotides. In some embodiments, the region is a window of 25-45 nucleotides. In some embodiments, the region is a window of 20-50 nucleotides. In some embodiments, the region is a window of 20-45 nucleotides. In some embodiments, the region is a window of 22 nucleotides.

The 16S rRNA is a component of the prokaryotic ribosome 30S subunit. Eukaryotic cells do not have 16S rRNAs, but as the chloroplast organelle is or prokaryotic origin it does contain a 16S rRNA. In some embodiments, the 16S rRNA is a chloroplast 16S rRNA. In some embodiments, the 16S rRNA is the 16S rRNA of a chloroplast of a specific plant cell. It will be understood that the sequence of the 16S rRNA is unique to each plant cell's chloroplasts. 16S rRNA sequences can be found, for example, at ncbi.nlm.nih.gov/refseq/targetedloci/16S_process/. The method described herein can be performed with any 16S rRNA sequence.

In some embodiments, the sum is multiplied by a constant. In some embodiments, the constant is a correction. In some embodiments, the constant is a parameter that can be optimized. In some embodiments, the constant is selected from 0, 1, 2, 3, 4, 5, 6, 7, 8, 9 and 10. Each possibility represents a separate embodiment of the invention. In some embodiments, the constant is 7. In some embodiments, the constant is 9. In some embodiments, the constant is 0.

Methods and programs for predicting hybridization are well known in the art and any such method or program can be used. In some embodiments, the prediction of hybridization is performed as described hereinbelow.

Methods, programs and websites for calculating free folding energy are well known in the art and any may be used. In some embodiments, the free folding energy of the mRNA region is calculated with mRNAfold. In some embodiments, the free folding energy of the rRNA region is calculated with mRANfold. In some embodiments, the free folding energy of the hybridized regions is calculated with RNAcofold. In some embodiments, calculating free folding energy is performed as described hereinbelow. In some embodiments, calculating free folding energy comprises calculating relative free folding energy. In some embodiments, relative free folding energy comprises calculating free folding energy for a null model. In some embodiments, the null model is for the region. In some embodiments, the null model is for a sequence of the region. In some embodiments, the null model is for a sequence aligned with the region. In some embodiments, the relative free folding energy is the difference between the free folding energy of the sequence and the null model. In some embodiments, the relative free folding energy is the difference between the free folding energy of the region and the null model.

In some embodiments, a lower combined free folding energy as compared to a sum of the target free folding energy and the rRNA free folding energy indicates translational initiation. In some embodiments, lower is more negative. It will be understood that the skilled artisan that the lower or more negative a free folding energy is the more stable the molecule is. Thus, a lower combined energy indicates the combined configuration is more stable, or energetically favorable, that the configuration of the two molecules separate. In some embodiments, indicating translational initiation is indicated the location of translational initiation. In some embodiments, the location is the site. In some embodiments, translational initiation is translation initiation. In some embodiments, a combined free folding energy lower by more than a predetermined threshold indicates translational initiation. In some embodiments, the magnitude by which the combined free folding energy is lower is proportionate to the efficiency of translation initiation. In some embodiments, the lower the combined energy the more efficient the initiation. In some embodiments, the greater the difference between the combined free folding energy and the sum of the target and rRNA free folding energy the more efficient the initiation.

In some embodiments, the method comprises calculating for a plurality of regions in the UTR. In some embodiments, the method comprises performing steps a-c for a plurality of regions within the 5′ UTR. In some embodiments, the method comprises calculating for a plurality of regions in the rRNA. In some embodiments, the method comprises performing steps a-c for a plurality of regions within the rRNA. In some embodiments, a plurality is at least 2, 3, 4, 5, 6, 7, 8, 9, 10, 20, 25, 30, 40, 50, 60, 70, 75, 80, 90, 100, 150, 200, 250, 300, 400, 500, 600, 700, 750, 800, 900 or 1000 regions. Each possibility represents a separate embodiment of the invention. In some embodiments, the plurality of regions comprises enough regions to examine all of the UTR. In some embodiments, all of the UTR is all of the 5′ UTR. In embodiments, examining all of the UTR comprises regions that cover all of the UTR. In some embodiments, the method of examining the UTR is as described hereinbelow. In some embodiments, the plurality of regions is determined by shifting the window corresponding to the region by 10, 9, 8, 7, 6, 5, 4, 3, 2, or 1 nucleotide and examining a new window/region. Each possibility represents a separate embodiment of the invention. In some embodiments, the plurality of regions is determined by shifting the window corresponding to the region by 5 nucleotides and examining a new window/region. In some embodiments, the plurality of region is determined by shifting the start of the first region by 10, 9, 8, 7, 6, 5, 4, 3, 2, or 1 nucleotide and reperforming the determination of windows/region. Each possibility represents a separate embodiment of the invention. In some embodiments, the plurality of region is determined by shifting the start of the first region by 1 nucleotide and reperforming the determination of windows/region. In some embodiments, the start of the windows is shifted through the first 100, 90, 80, 70, 60, 50, 40, 30, 25, 20, 15, 10 or 5 nucleotides. Each possibility represents a separate embodiment of the invention. In some embodiments, the start of the windows is shifted through the first 50 nucleotides. In some embodiments, the shift is upstream. As all of the positioning is calculated from the translational start site (TSS, e.g., the start codon) the shifting is done upstream, that is the 5′ direction, away from the TSS.

In some embodiments, the method comprises selecting from the plurality of regions in the mRNA the region with the lowest combined free folding energy. In some embodiments, the method comprises selecting from the plurality of regions in the mRNA the region with the greatest difference between the combined free folding energy and the sum of the target free folding energy and the rRNA free folding energy. In some embodiments, greatest difference is greatest negative difference (i.e., the combined free folding energy is lower). In some embodiments, selecting a region comprises selecting more than one region. In some embodiments, the more than one region is more than one adjacent regions. In some embodiments, the more than one region are all regions with combined free folding energy below a predetermined cutoff. In some embodiments, the more than one region are all regions with combined free folding energy that varies by less than a predetermined amount or percentage. In some embodiments, the more than one region are adjacent or contiguous with the selected region. It will be understood by a skilled artisan that translation initiation may occur over an area of sequence that spans a region, thus if contiguous regions all show similar combined free folding energy (or difference in combined free folding energy as compared to the sum of the target and rRNA) or all have sufficiently low combined free folding energy (e.g., all are efficient for translation initiation) the region of initiation may be larger than just one window.

In some embodiments, the selecting further comprises selecting a region with a free folding energy below a predetermined threshold. In some embodiments, a region is a combined region. In some embodiments, the free folding energy is relative free folding energy. In some embodiments, relative is relative to the null model. In some embodiments, the selecting comprises selecting a region comprising a relative free folding energy that is significantly lower than the relative free folding energy of an adjacent region. In some embodiments, the selecting comprises selecting a region comprising a relative free folding energy that is significantly higher than the relative free folding energy of an adjacent region. It will be understood that for selecting regions of translation initiation the region will have a lower relative free folding energy and that for regions of translation termination the region will have a higher relative free folding energy. In some embodiments, significantly is statistically significantly. In some embodiments, significantly is by more than a predetermined threshold. In some embodiments, adjacent is adjacent upstream of the region. In some embodiments, adjacent is adjacent downstream of the region. In some embodiments, an adjacent region is both regions adjacent to the region (i.e., both the upstream adjacent and downstream adjacent regions). In some embodiments, the selecting is thus selecting a region with a conserved structure. In some embodiments, the conserved structure is conserved in the plurality of species. In some embodiments, the conserved structure is conserved in all species of the plurality of species.

In some embodiments, the method is a method of predicting protein expression from the mRNA. In some embodiments, protein expression is in the cell. In some embodiments, protein expression is in the organelle. In some embodiments, protein expression is in a chloroplast. In some embodiments, the predicted translation initiation efficiency is proportional to the predicted protein expression. It will be understood by a skilled artisan that while there are other factors that will impact protein levels the more efficient the initiation of translation the more abundant the protein will be. Thus, by predicting initiation efficiency protein expression can also be predicted. In some embodiments, the greater the difference between the combined free folding energy and the sum of the target free folding energy and the rRNA free folding energy the greater the expression of the protein.

In some embodiments, the method further comprises confirming the prediction. In some embodiments, the confirming is by comparing the predicted translation initiation efficiency to received levels of the mRNA. In some embodiments, the confirming is by comparing the predicted translation initiation efficiency to received levels of the protein. In some embodiments, the received levels of the protein are receiving a measure of protein expression levels. In some embodiments, the protein expression levels are of a protein encoded by the mRNA. In some embodiments, the protein expression levels are of a protein translated form the mRNA. In some embodiments, the protein expression levels are in the cell. In some embodiments, the protein expression levels are in the organelle. In some embodiments, the protein expression levels are in the chloroplast. In some embodiments, the confirming is by comparing the predicted protein expression to received levels of the mRNA. In some embodiments, the confirming is by comparing the predicted protein expression to received levels of the protein. In some embodiments, the confirming is by comparing the predicted protein expression to the received measure. In some embodiments, the received levels are from the cell. In some embodiments, the received levels are from the organelle. In some embodiments, the received levels are from the chloroplasts.

In some embodiments, the received expression levels are approximated levels. In some embodiments, the approximation is by an estimate based on the mRNA sequence. Methods of approximating protein expression based on mRNA sequence are well known and any such method may be used. It is understood that the use of rare codons and slowly translating codons will lead to reduced protein expression and that common/quickly translating codons will lead to increased protein expression. Any measure that captures the sequence effect on translation may be used. In some embodiments, the approximating is by codon adaptation index (CAI) in the mRNA. In some embodiments, the received expression levels are approximated by the CAI of the mRNA. In some embodiments, the approximating comprises a method employing CAI. In some embodiments, the CAI is used to approximate the protein expression from the mRNA.

In some embodiments, the method further comprises correlating the predicted expression levels to the measure. In some embodiments, the method further comprises correlating the predicted expression levels to the received levels. In some embodiments, the method further comprises optimizing the correlation. In some embodiments, the optimizing comprises providing a parameter from the method and altering that parameter. In some embodiments, the optimizing comprises altering a parameter from the method of the invention such that the predicted levels more closely correlate with the received levels. In some embodiments, the optimizing comprises altering a parameter from the method of the invention such that the predicted initiation efficiency more closely correlates with the received levels. In some embodiments, the optimizing comprises providing a plurality of mRNAs of proteins expressed in the chloroplast, selecting a subgroup of the plurality as a training set and a subgroup of the plurality as a test set, selecting a parameter that optimizes correlation between the predicted expression levels in the training set to the measure of protein expression and validating the parameter in the test set. In some embodiments, the parameter is region size. In some embodiments, size is length. In some embodiments, region size is window size. In some embodiments, the parameter is 5′ UTR region size. In some embodiments, the parameter is 16S rRNA region size. In some embodiments, the parameter is region start position. In some embodiments, start position is start position in the UTR. In some embodiments, start position is start position in the rRNA. In some embodiments, the parameter is a correction factor. In some embodiments, the correction factor is a correction in the calculating. In some embodiments, the correction factor is a correction the approximating. In some embodiments, the correction factor is a correction in the correlating. In some embodiments, the correction factor is a coefficient. In some embodiments, the correction factor corrects the second-order aspects of translation regulation. In some embodiments, the correction factor is applied to the sum of the target and rRNA free folding energy. In some embodiments, the correction factor is a coefficient by which the sum is multiplied. In some embodiments, the method further comprises multiplying the sum of the target and rRNA free folding energy by a correction factor. In some embodiments, optimized parameters are selected from those provided in Table 1.

In some embodiments, the plurality of regions comprises enough regions to examine all of the 16S rRNA. In some embodiments, the plurality of regions comprises enough regions to examine all of the 16S rRNA tail. In some embodiments, the tail is the 3′ terminal 50, 100, 150, 200, 250, 300, 350, 400, 450, 500, 550, 600, 650, 700 or 750 nucleotides. Each possibility represents a separate embodiment of the invention. In embodiments, examining all of the 16S RNA comprises regions that cover all of the 16S RNA. In embodiments, examining all of the 16S RNA tail comprises regions that cover all of the 16S RNA tail. In some embodiments, the method of examining the UTR is as described hereinbelow. In some embodiments, the plurality of regions is determined by shifting the window corresponding to the region by 1 nucleotide and examining a new window/region.

In some embodiments, the database is a database of sequences. In some embodiments, the sequences are sequences of the mRNA. In some embodiments, the mRNA is the mRNA in chloroplasts. In some embodiments, the sequences are of the mRNA from different species of chloroplasts. In some embodiments, different species of chloroplasts are chloroplast from different species of plants. In some embodiments, different species are a plurality of species. In some embodiments, the database comprises at least 2, 3, 5, 7, 10, 15, 20, 30, 40, 50, 100, 200, 300, 400, 500, 1000, 2000, 3000, 4000, 4100, 4200, 4300, 4400, 4500, 4600, or 5000 sequences. Each possibility represents a separate embodiment of the invention. In some embodiments, a plurality of species comprises at least 2, 3, 5, 7, 10, 15, 20, 30, 40, 50, 100, 200, 300, 400, 500, 1000, 2000, 3000, 4000, 4100, 4200, 4300, 4400, 4500, 4600, or 5000 species. Each possibility represents a separate embodiment of the invention. In some embodiments, a plurality of species is at least 10 species. In some embodiments, a plurality of species is at least 4600 species. In some embodiments, the database comprises at least 4600 mRNAs. In some embodiments, the database comprises at least 10 mRNAs.

Aligning sequences based on similarity is well known in the art and numerous computer programs, websites and applications can perform such an alignment and produce an MSA. In some embodiments, the alignment is based on sequence similarity. In some embodiments, the alignment is based on sequence homology. In some embodiments, the MSA produces a consensus sequence. In some embodiments, the consensus sequence is the consensus sequence of the mRNA. In some embodiments, the free folding energy for a region in the consensus sequence of the mRNA is calculated to produce a target free folding energy. In some embodiments, the free folding energy for a region of the mRNA and each sequence aligned with the region is calculated to produce target free folding energies. In some embodiments, the aligned sequences are as aligned in the MSA. In some embodiments, the target free folding energies are averaged to produce a target free folding energy.

In some embodiments, the free folding energy is calculated for a region. In some embodiments, the free folding energy is calculated for a plurality of regions. In some embodiments, the free folding energy is calculated for all regions. In some embodiments, all regions are all regions in the mRNA. In some embodiments, all regions are all regions in the UTRs of the mRNA. In some embodiments, all regions are all regions in the 5′ UTR of the mRNA. In some embodiments, all regions are all regions in the 3′ UTR of the mRNA. In some embodiments, all regions are all regions in the coding region of the mRNA.

In some embodiments, a region in which the target free folding energy is lower than a free folding energy in a null model is selected. In some embodiments, regions in which the target free folding energy is lower than a free folding energy in a null model are selected. In some embodiments, the average target free folding energy is lower than the null model. In some embodiments, the consensus target free folding energy is lower than the null model. In some embodiments, a plurality of the free folding energies are lower than the null model. In some embodiments, the plurality is at least 50, 55, 60, 65, 70, 75, 80, 85, 90, 95, 97, 99 or 100% of the free folding energies. Each possibility represents a separate embodiment of the invention. In some embodiments, the selected region is a region of conserved mRNA structure. In some embodiments, all regions that are lower than the null model are selected. In some embodiments, all regions that are lower than the null model by at least a predetermined threshold are selected.

Calculation of a null model is well known in the art and any such method may be used. In some embodiments, the null model is calculated as described hereinbelow. In some embodiments, the null model maintains the amino acid sequence of the protein encoded by the mRNA. In some embodiments, the null model maintains the codon usage in the mRNA. In some embodiments, the null model maintains the GC content in the mRNA. In some embodiments, the null model maintains evolutionary conservation in the mRNA. In some embodiments, the null model comprises shuffling the CAI between the mRNAs of the database. In some embodiments, CAI is Z-scored CAI.

In some embodiments, translation initiation efficiency in the selected region is predicted. In some embodiments, translation initiation efficiency is predicted by a method of the invention. In some embodiments, translation initiation efficiency is predicted by a method comprising:
a. calculating a free folding energy for the selected region to produce a target free folding energy;
b. calculating a free folding energy for a region of a 16S ribosomal RNA (rRNA) to produce an rRNA free folding energy; and
c. calculating a free folding energy for the selected region hybridized to the 16S rRNA region to produce a combined free folding energy;
thereby predicting translation initiation.

In some embodiments, the method further comprises selecting a region as a region initiating translation if the combined free folding energy is lower than a sum of the target free folding energy and the rRNA free folding energy. In some embodiments, regions in the 5′ UTR are selected as initiating translation. In some embodiments, only regions from the 5′ UTR are selected as initiating translation. In some embodiments, regions from the 3′ UTR are not selected as initiating translation. In some embodiments, the region initiating translation is the determine region regulating translation. In some embodiments, the method further comprises selecting a region as a region terminating translation if the combined free folding energy is higher than a sum of the target free folding energy and the rRNA free folding energy. In some embodiments, the region terminating translation is the determine region regulating translation. In some embodiments, regions in the 3′ UTR are selected as terminating translation. In some embodiments, only regions from the 3′ UTR are selected as terminating translation. In some embodiments, regions from the 5′ UTR are not selected as terminating translation. In some embodiments, lower than is lower than by a predetermined threshold. In some embodiments, higher than is higher than by a predetermined threshold.

In some embodiments, all possible regions are evaluated. In some embodiments, all regions are all regions in the UTRs. In some embodiments, all regions are all regions in the 5′ UTR. In some embodiments, all regions are all regions in the 3′ UTR. In some embodiments, all regions does not comprise a region comprising the Shine-Dalgarno sequence. In some embodiments, all regions that initiate translation are selected. In some embodiments, all regions that terminate translation are selected. In some embodiments, adjacent regions that initiate translation are combined to produce a complete initiating region. In some embodiments, adjacent regions that terminate translation are combined to produce a complete terminating region. In some embodiments, adjacent regions are contiguous regions. In some embodiments, all contiguous regions that initiate translation are combined. In some embodiments, all contiguous regions that terminate translation are combined.

In some embodiments, selected regions initiating or terminating translation are conserved functional mRNA structures. In some embodiments, the method is a method for determining secondary mRNA structure. In some embodiments, the secondary mRNA structure is conserved secondary mRNA structure. In some embodiments, the secondary mRNA structure is structures that initiate translation. In some embodiments, the secondary mRNA structure is structures that terminate translation. In some embodiments, the structure is the structure of the selected region. In some embodiments, structure is mRNA structure. In some embodiments, structure is secondary structure. In some embodiments, structure is folding. In some embodiments, folding is mRNA folding. Methods, programs and websites for determining/predicting/approximating mRNA folding are well known and any may be employed for determining the structure from the sequence of the region. In some embodiments, RNAfold is used for determining the structure. In some embodiments, RNAfold is RNAalifold. In some embodiments, RNAfold is Viennafold.

By another aspect, there is provided a method of modulating translation of a target mRNA, the method comprising determining a region regulating translation and
a. generating the determined region in the target mRNA; or
b. abolishing the determined region in the target mRNA;
thereby modulating translation of a target mRNA

By another aspect, there is provided a method of modulating translation of a target mRNA, the method comprising generating in the target mRNA a region folding into a secondary structure selected from those provided in Table 3 or abolishing in the target mRNA a secondary structure selected from those provided in Table 3, thereby modulating translation of a target mRNA.

By another aspect, there is provided a method of modulating translation of a target mRNA, the method comprising generating in the target mRNA a region folding into a secondary structure selected from those provided in Table 3 or abolishing in the target mRNA a secondary structure selected from those provided in Table 3, thereby modulating translation of a target mRNA.

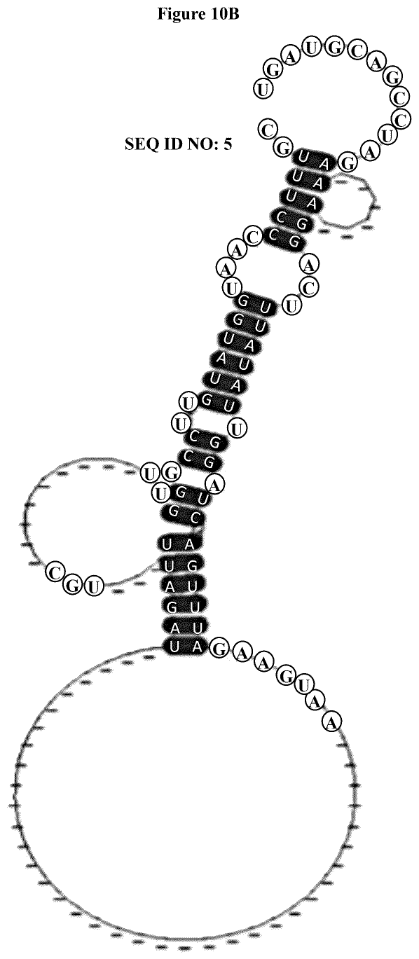

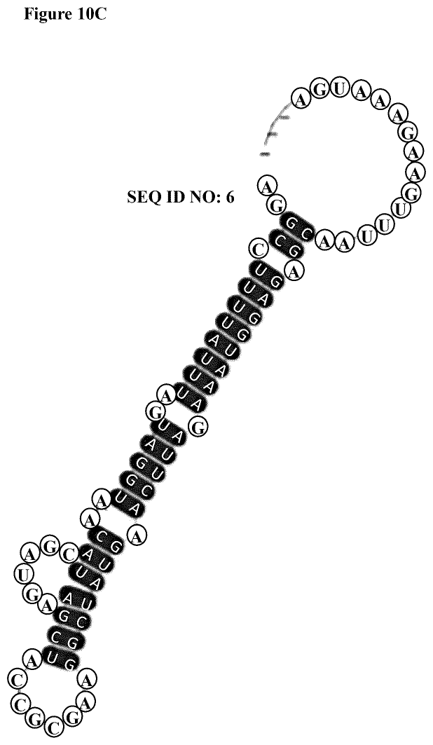

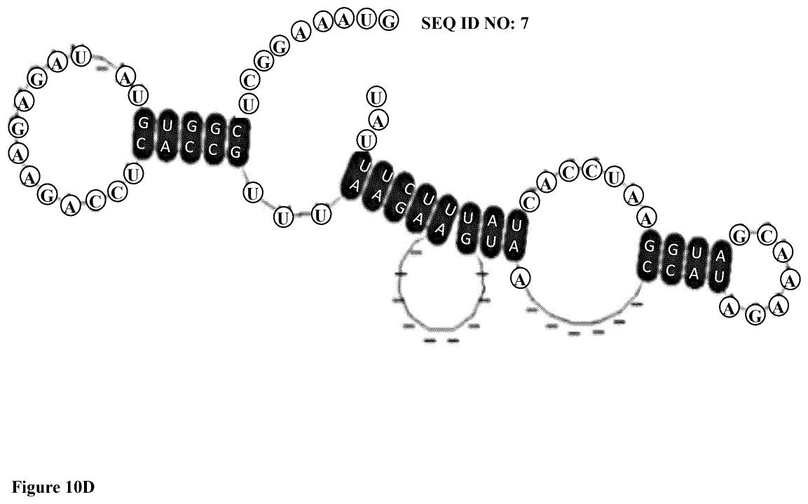

By another aspect, there is provided a method of modulating translation of a target mRNA, the method comprising generating in the target mRNA a region folding into a secondary structure selected from those provided in FIG. 10A-D or abolishing in the target mRNA a secondary structure selected from those provided in FIG. 10A-D, thereby modulating translation of a target mRNA.

By another aspect, there is provided a method of modulating translation of a target mRNA, the method comprising generating in the target mRNA a region comprising or consisting of a sequence selected from SEQ ID NO: 1 and 3-7.

By another aspect, there is provided a method of modulating translation of a target mRNA, the method comprising generating in the target mRNA a region folding into a secondary structure produced by a sequence selected from SEQ ID NO: 1 and 3-7.

In some embodiments, the sequence is SEQ ID NO: 1. In some embodiments, the sequence is SEQ ID NO: 3. In some embodiments, the sequence is selected from SEQ ID NO: 4-7. In some embodiments, the sequence is SEQ ID NO: 4. In some embodiments, the sequence is SEQ ID NO: 5. In some embodiments, the sequence is SEQ ID NO: 6. In some embodiments, the sequence is SEQ ID NO: 7. In some embodiments, the generating is within the 3′ UTR. In some embodiments, the generating is within the 5′ UTR. In some embodiments, the generating is proximal to the translational stop site. In some embodiments, the generating is proximal to the translational start site. In some embodiments, proximal to is adjacent to.

In some embodiments, the target mRNA is a chloroplast mRNA. In some embodiments, the target mRNA is an mRNA translated in chloroplasts. In some embodiments, the target mRNA is an mRNA which does not naturally comprise the determined region. In some embodiments, the target mRNA is not the mRNA from which the determined region was determined. In some embodiments, the target mRNA is not the mRNA from which the determined region was selected. In some embodiments, the target mRNA is the mRNA from which the determined region was determined. In some embodiments, the target mRNA is the mRNA from which the determined region was selected. In some embodiments, the generating occurs in an mRNA that is not the mRNA from which the region was determined. In some embodiments, the abolishing occurs in an mRNA that is the mRNA from which the region was determined. In some embodiments, the abolishing occurs in an mRNA that is not the mRNA from which the region was determined but which also comprises the structure. In some embodiments, comprising the structure is comprising the region.

In some embodiments, the secondary structure is provided in Table 3. A skilled artisan provided with Table 3 and the RNAalifold program can reproduce the structures provided in this table. In Table 3 sequences are provided with Ns that can be any nucleic acid base. Further, the Ns are given as Nx, where “x” represents the number of bases. In some embodiments, x is at least 1. In some embodiments, x is 1-20. In some embodiments, x is 1-50. In some embodiments, x is 1-100. In some embodiments, x is 1-200. In some embodiments, the RNAalifold output provides the exact number of Xs at each position. It will be understood that the Xs need not be all the same, but can be different and indeed any combination of bases. This can be seen in FIG. 10B. FIG. 10B shows the structure produced by the folding of SEQ ID NO: 2. However, SEQ ID NO: 2 could also be provided as UGAUGCAGCCTAGAA(X)nAGGACUUUAUAUUGGAUCAGUUUAGAAGUAA(X)n UAGAUU(X)nUGC(X)nUUGGGCCUUGUAUGGUAACCCUUUGC as it will be understood that the exact number of basepairs is not essential in the loops that are not sequence specific. Further, it will be understood that regions of basepairing (e.g., stem regions) are not sequence specific but rather any basepairs can be used. That is, the creation of the specific secondary structure is what is important for regulating translation and not the specific sequence. As such, other sequences that produce the same secondary structure can be used. These structures are also provided, and visualized, in Ezra and Tuller, “Modeling the effect of rRNA-mRNA interactions and mRNA folding on mRNA translation in chloroplasts”, Comput. Struct. Biotechnol. J., 2022 May 18; 20:2521-2538, in Supplementary Data 3. Further, the structures are provided in FIG. 16 of US Provisional Patent Application U.S. 63/337,113.

In some embodiments, generating comprises insertion of the determined region in the mRNA. In some embodiments, generating comprises insertion of the region folding into the secondary structure in the mRNA. In some embodiments, generating comprises mutation of a region of the target mRNA to produce the determined region. In some embodiments, generating comprises mutation of a region of the target mRNA to produce the region folding into the secondary structure. In some embodiments, generating comprises insertion of the determined region into a DNA encoding the mRNA. In some embodiments, generating comprises insertion of the region folding into the secondary structure into a DNA encoding the mRNA. In some embodiments, generating comprises mutation of a region of a DNA encoding the target mRNA to produce the determined region. In some embodiments, generating comprises mutation of a region of a DNA encoding the target mRNA to produce the region folding into the secondary structure.

In some embodiments, the generating is at a location in the target mRNA that corresponds to the location of the determined region in the mRNA from which it was determined. In some embodiments, the generating is at a location in the target mRNA that corresponds to the location of the region folding into secondary structure in the mRNA from which it was identified. In some embodiments, the location in the mRNA from which it was identified and the location from which it was determined is selected from a location provided in Table 2. In some embodiments, a corresponding region is a 5′ UTR to a 5′ UTR. In some embodiments, the determined region is located in its original mRNA in a 5′ UTR and is generated in a 5′ UTR of the target mRNA. In some embodiments, the determined region is located in its original mRNA in a 3′ UTR and is generated in a 3′ UTR of the target mRNA. In some embodiments, the original mRNA is the mRNA in which the region was determined. In some embodiments, the original mRNA is the mRNA in which the region folding into secondary structure was identified. In some embodiments, a corresponding region is a 3′ UTR to a ‘3 UTR. In some embodiments, a corresponding region is approximately the same distance from the TSS. In some embodiments, a corresponding region is approximately the same distance from the translational termination site (TTS, e.g., the stop codon). In some embodiments, approximately is within 1, 5, 10, 15, 20, 25, 30, 35, 40, 45, 50, 60, 70, 75, 80, 90, 100, 150, 200, 250, 300, 350, 400, 450, 500 or 1000 nucleotides. Each possibility represents a separate embodiment of the invention.

In some embodiments, abolishing comprises deleting the determined region. In some embodiments, abolishing comprises deleting the region folding into the secondary structure. In some embodiments, abolishing comprises mutating the determined region. In some embodiments, abolishing comprises mutating the region folding into the secondary structure. In some embodiments, mutating is mutating at least one nucleotide. In some embodiments, mutating is mutating at least 1, 2, 3, 4, 5, 6, 7, 8, 9, 10, 11, 12, 13, 14, 15, 20, 25, 30, 35, 40, 45 or 50 nucleotides. Each possibility represents a separate embodiment of the invention. IN some embodiments, mutating is mutating a sufficient number of nucleotides so as to abolish the secondary structure. In some embodiments, secondary structure is secondary structure in the determined region. In some embodiments, the mutating changes the local folding energy of the determined region in the mRNA by at least a predetermined threshold. In some embodiments, the mutating changes the local folding energy of the determined region folding into the secondary structure by at least a predetermined threshold.

In some embodiments, the determining a region regulating translating is by a method of the invention. In some embodiments, the determining a region regulating translating is as described hereinabove.

By another aspect, there is provided a nucleic acid molecule comprising the determined or selected region.

By another aspect, there is provided an mRNA molecule produced by a method of the invention.

By another aspect, there is provided a DNA molecule encoding the mRNA molecule of the invention.

In some embodiments, the nucleic acid molecule is an mRNA. In some embodiments, the mRNA molecule comprises a heterologous mRNA sequence. In some embodiments, the mRNA molecule is not an mRNA that naturally comprises the selected or determined region. In some embodiments, the mRNA comprises an open reading frame. In some embodiments, the open reading frame does not encode the same protein as the open reading from of the mRNA from which the region was determined/selected.

As used herein, the term “mRNA” is used to refer to modified and unmodified RNA including both a coding region and a noncoding region. As used herein, the phrases “coding sequence” and “coding region” of an mRNA are interchangeably used herein to refer to the region that when translated results in the production of an expression product, such as a polypeptide, protein, or enzyme.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

In some embodiments, the DNA molecule comprises an open reading frame encoding the nucleic acid molecule of the invention. In some embodiments, the DNA molecule comprises an open reading frame encoding the mRNA of the invention. In some embodiments, the DNA molecule is a vector. In some embodiments, the vector is an expression vector. In some embodiments, the DNA molecule comprises at least one regulatory element. In some embodiments, the regulatory element is a transcriptional regulatory element. In some embodiments, the vector is configured to express in chloroplasts. In some embodiments, the regulatory element is active in chloroplasts. In some embodiments, active is induces transcription. In some embodiments, the regulatory element is operatively linked to the open reading frame. In some embodiments, the regulatory element is a promoter.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZcoSV2 (±), pSccTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

The analyzed organisms: All the chloroplasts' genomes (4603) were downloaded from NCBI according to their accession number which were extracted from NCBI website (ncbi.nlm.nih.gov/).

### Genes' Regions, 5′UTR, ORF, 3′UTR:

The ORF for every gene was taken according to the positions in the genome listed in the information from NCBI. The 5′UTR was determined from the end of the previous gene's ORF positions in the genome until the start of the ORF of the gene, and 3′UTR determined from the end of the ORF of the gene until the start of the following gene's ORF positions in the genome. The UTRs in almost all the genomes that were analyzed herein are not annotated. Since it is not clear where the 5′UTR of every gene begins we took the nucleotides from the end of the ORF of the previous gene in the chromosome until the last nucleotide before the start codon as the 5′UTR raw data, and the nucleotides of the 3′UTR of every gene was taken from end of the ORF until the start codon of the following gene in the chromosome (with median predicted 5′UTR length which is 334 nucleotides and median 3′UTR which is 262). However, we performed a procedure to infer the relevant features of the model (i.e., the length and position of the window upstream of the ORF) via fitting the model to gene expression (as elaborated in Materials and Methods section 5.7). Indeed, the inferred relevant UTR was usually short (a few dozen nucleotides) according to the results. The fact that the model has high predictive power supports the conjecture that our inference is meaningful.

### Protein Abundance and Ribosomal Profiling Values:

Chlamydomonas reinhardtii PA values information was downloaded from Strenkert et al., 2019, “Multiomics resolution of molecular events during a day in the life of Chlamydomonas” herein incorporated by reference in its entirety, for every gene the PA was calculated by the average of the PAs measured at all hours of the day, in addition PA values were downloaded from PaxDB out of C. reinhardtii—Whole organism (from Wang et al., 2014, “The global phosphoproteome of Chlamydomonas reinhardtii reveals complex organellar phosphorylation in the flagella and thylakoid membrane”, herein incorporated by reference in its entirety). The PA values from every source were normalized such that both sources have the same average of 1 by dividing the average PA. The PA values that were taken for calculations in this study were the average PA from the two normalized sources for every gene.

### CAI Calculation:

CAI, codon adaptation index, is a computational method of predicting the expression level of a gene based on its codon sequence.

The steps for calculating this index are:
a) Calculating weights for every codon i according to the frequency of this codon in a reference set (the highly expressed genes of the current genome) divided by the maximum frequency of the codon that encodes for the same amino acid, according to equation (1):

$${w}_{i} = \frac{{x}_{i}}{max ( x )} \qquad ( 1 )$$

Where wᵢ refers to the weight of codon i, xᵢ refers to the repetitions amount in the reference set, max (x) refers to the maximal synonymous codon's repetitions.
b) Calculating CAI for every gene in length of L codons is by the geometrical mean of all the codons' weights in the sequence according to equation (2):

$${CAI}_{g e n e} = {( {\prod }_{i = 1}^{L} {w}_{i} )}^{\frac{1}{L}} \qquad ( 2 )$$

DCBS—is a measure of the strength of the codon usage bias (CUB) of the gene, and is calculated based on the following equations:
the directional codon bias (DCB) of a codon triplet xyz:

$${d}_{x y z} = max ( \frac{f ( x , y , z )}{{f}_{1} ( x ) \cdot {f}_{2} ( y ) \cdot {f}_{3} ( z )} , \frac{{f}_{1} ( x ) \cdot {f}_{2} ( y ) \cdot {f}_{3} ( z )}{f ( x , y , z )} ) \qquad ( 3 )$$

The DCBS of a gene of length L codons:

$$DCBS = \frac{{\sum }_{i = 1}^{L} {d}_{x y z}}{L} \qquad ( 4 )$$

CAI was calculated for every gene in every chloroplast's genome according to the following steps:
a) DCBS was calculated for every gene, 20% of the genes with the highest DCBS were taken as the reference set for CAI calculations.
b) CAI scores were calculated for every gene.
c) The CAI scores were normalized for every chloroplast by replacing them with a Z-score according to equation (3):

$$Zscore = \frac{{x}_{i} - mean ( x )}{std ( x )} \qquad ( 3 )$$

Where x represents the entire group of values (i.e., CAI values related to all the genes in the genome), and xᵢ is one value from the group (i.e., the CAI of one gene).

### Orthologous Groups:

In order to create orthologous groups of chloroplast's genes we first grouped together genes from different chloroplasts with the same gene product according to the chloroplasts genes names proposal. The homology between every couple of genes in every group was estimated by BLAST, and pairs of genes that did not meet with the following conditions were eliminated from the group: E value higher than 10⁻⁵ (considered as significant enough based on empirical studies) and identity percent lower than 50%.

After this elimination step, a graph was calculated for every group by the remaining couples of genes that are considered to be similar enough. In this graph, nodes are genes in the group and an edge between a couple of genes means that these genes are similar enough according to the conditions explained above. The number of edges that are related to each gene in the graph was calculated, and genes that are connected to less than 40% of the rest of the group members were eliminated from the group. At last step, genes with lengths significantly different than the mean gene length of the group (probably because of false sequence annotations) were eliminated. This included gene length shorter than 65% of the group's mean gene length and gene length longer than 140% of the group's mean gene length.

As a result, we generated 77 orthologous groups from the genes in the database with an average of 4,325 genes per group. Based on this procedure, a gene is added to an orthologous group if its similarity to the rest of the group is above a certain threshold. This threshold (an edge in the graph) is sensitive enough to detect homology even for a pair of organisms that are not evolutionary close. Thus, since the threshold is absolute and not relative, we do not expect to miss orthologs due to the distribution of organisms in the databases.

Positions with a Sufficient Number of Nucleotides in the MSA:

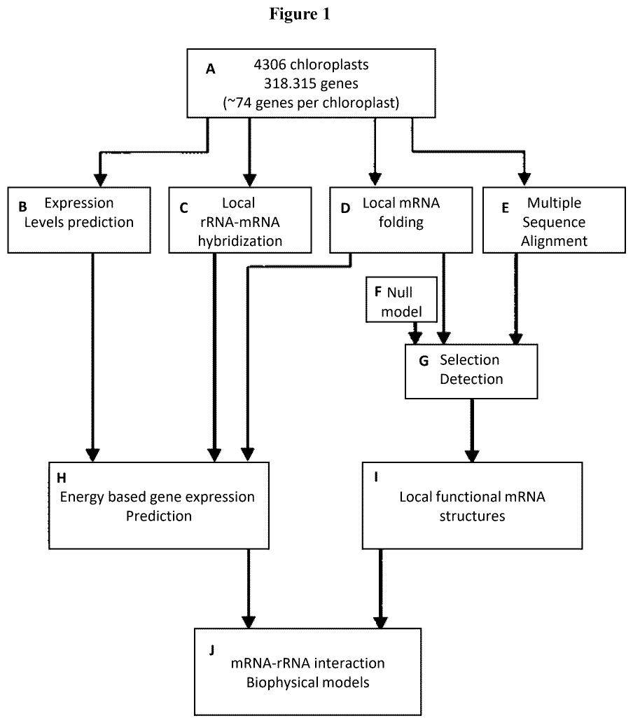

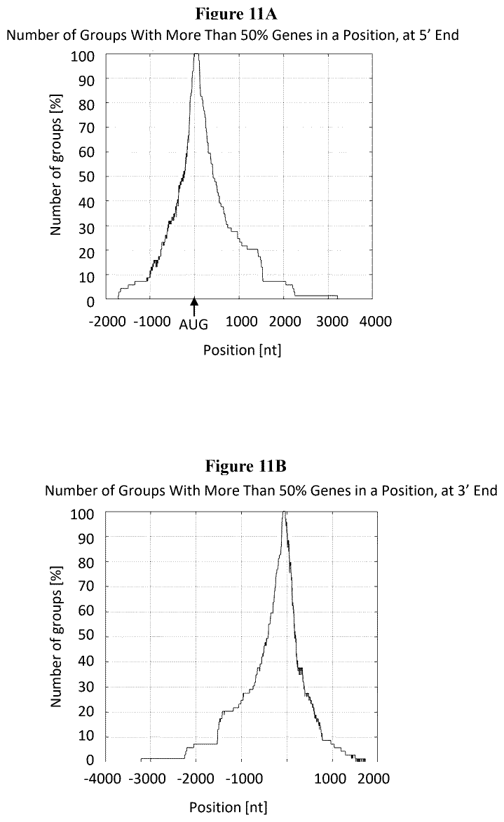

In This study we investigated the consensus functional secondary structures for different orthologous groups, the genes in the database vary in their length of the 5′UTR, ORF, and 3′UTR, therefore in order to calculate statistic for the results of all the orthologues groups' secondary structures according to position upon the mRNA we took into account only positions upon the mRNA's MSA that most of the groups have more than 50% genes that contribute a nucleotide in a position. In FIG. 11 there is the number of groups with more than 50% genes with a nucleotide for every position upon the mRNA′ MSA. FIG. 11A refers to the MSA of 5′UTR and ORF aligned to the start codon, and FIG. 1B to the MSA of ORF and 3′UTR aligned to the stop codon. From this figure, the positions to take into consideration for the folding statistics are 500 nucleotides upstream and downstream of the start codon, which there are above 50% groups with the condition mentioned, and 500 upstream and downstream of the stop codon.

### Minimum Free Energy Calculations:

The minimum free energies of different sequences (of mRNA and 16S rRNA) were calculated using ViennaRNA package which predicts the secondary structure of RNA sequences and provides the minimum free energy of the thermodynamic ensemble.

The analyses along this study used two types of energy calculations by ViennaRNA:
a) RNAfold-calculates the minimum free energy of an RNA sequence, used to calculate the following energies:
   I. mRNA fold-minimum free energy and secondary structure of an mRNA sequence.
   II. 16S fold-minimum free energy and secondary structure of a 16S rRNA sequence.
b) RNAcofold-predicts the secondary structure upon a dimer formation, used to calculate the following energy:
   III. Cofold of mRNA and 16S-minimum free energy of the hybridization between the mRNA sequence and the 16S rRNA sequence.
The lower and negative the minimum free energy is, the stronger the structure is folded.

### Energy-Based Model:

We conducted a minimum free energy-based model related to energies of the local mRNA-rRNA hybridization. The model is based on the biophysical model in which at first the mRNA and the 16S are folded in a self-folding structures with energy calculated by RNAfold, and the second stage is that the two sequences, the mRNA and the 16S rRNA, bind together and create a new folded structure, the hybridization energy is calculated by RNAcofold. The mRNA window inferred in the model is the portion of the 5′ of the mRNA (which can include both parts of the 5′ UTR and parts of the ORF) representing a fragment that (most) effects mRNA translation. This window is described by its length in nucleotides and the position in the transcript where this window starts; thus, the “start position” refers to the position, relative to the start codon, of the first nucleotide of the mRNA window sequence that interacts with the 16S sequence of the ribosome. For example, if the start position of the mRNA window is-15 with a window size of 35 nt, it means that the mRNA window that interacts with the 16S window according to the model is 35 nucleotides long, and it starts at the position of 15 nucleotides upstream of the start codon. The model, which is called “Energy based Translation Initiation Predictor” (ETIP), estimates whether the hybridization energy between the local sequence of the mRNA and the 16S rRNA is stronger than the folding energies of the mRNA and the 16S rRNA separately, if so then the probability that the sequences will bind together in order to initiate translation will be higher and the translation will be more efficient. The model actually estimates how much the stability of the mRNA and the 16S rRNA was improved by their hybridization. The energy model is calculated according to equation (4):

$${ETIP}_{i} = {cofold}_{{mRNA}_{i} \& 16 {S}_{j}} - C * ( {fold}_{16 {S}_{j}} + {fold}_{{mRNA}_{i}} ) \qquad ( 4 )$$

When i refers to the i'th gene in the database, 16S; is the 16S rRNA sequence of chloroplast j.

The aim of the correction factor C is to deal with and correct second-order aspects of translation regulation that are not directly considered in the model. The purpose of this energy-based model is to predict the gene translation initiation efficiency that is expected to be highly correlated with the PA value and therefore with the CAI scores of a gene.

In order to predict the Z-scores of the CAI scores we investigated what are the typical properties of the local structures of the mRNA and its interactions with the 16S rRNA, which optimizes the correlation between the energy value of a gene's local structures and its CAI score, hence we characterized the structures by four parameters:
1) mRNA window length.
2) Start position of the mRNA window on the 5′UTR of the gene.
3) 16S rRNA window length from the 3′ end.
4) The correction factor, C, of ETIP model

It was expected that genes with different products will have different translation initiation mechanisms and it will tend to be conserved among the different genes in a group; thus, the model constraint all genes from a certain group to have identical parameters.

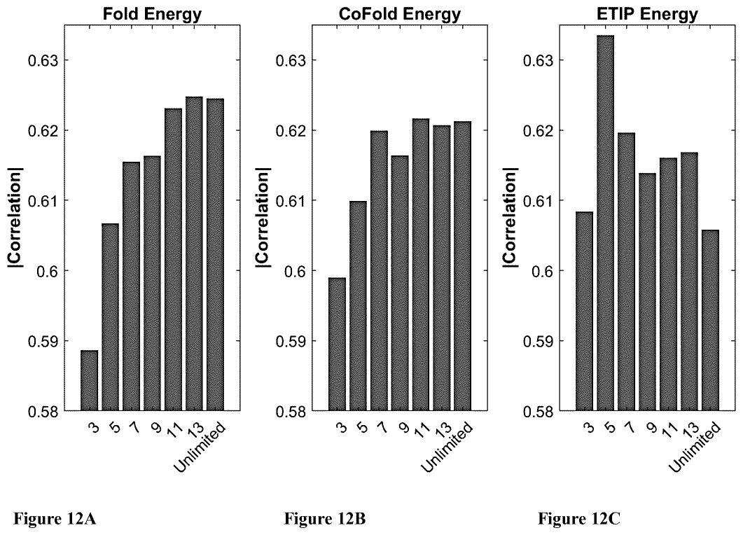

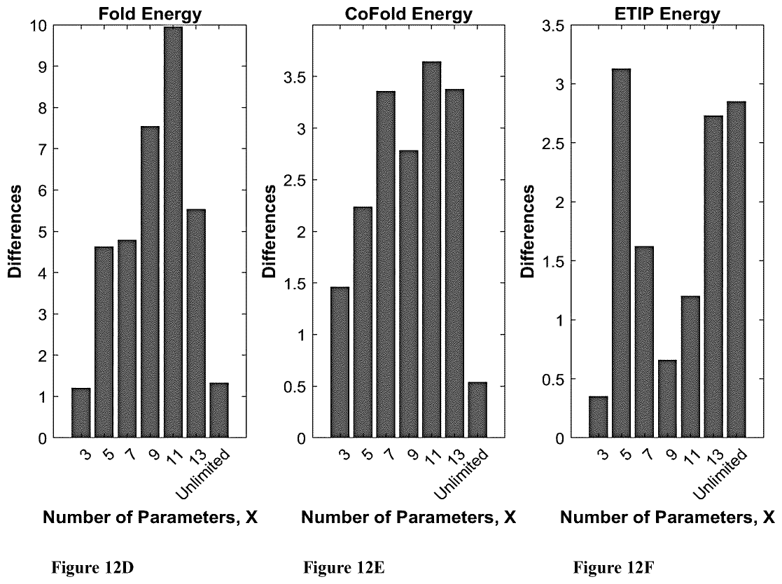

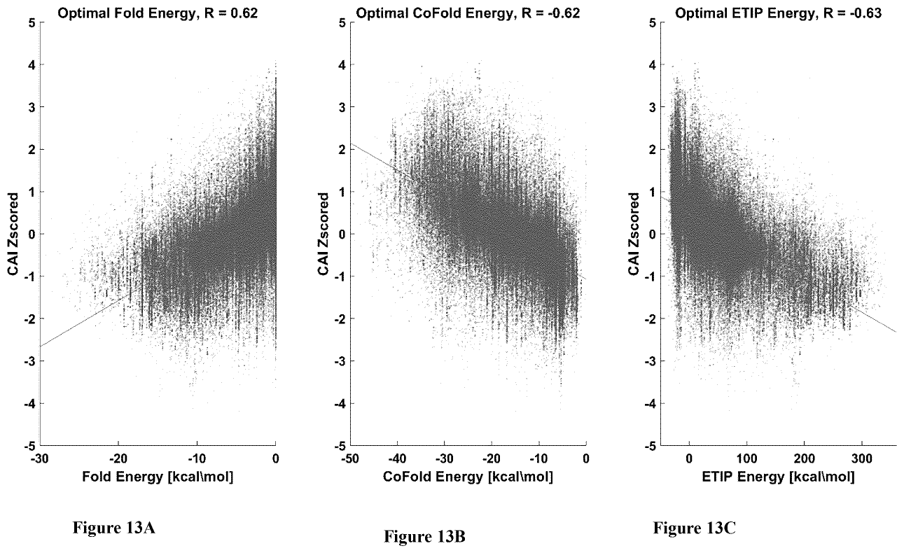

For comparison, we also conducted simpler energy-based models: one is based solely on the local folding energy of the mRNA which is predicted by ViennaRNA RNAfold algorithm, and the second one is based on the cofold energy of the hybridization between the local mRNA sequence and the local 16S rRNA sequence predicted by ViennaRNA RNAcofold algorithm. The mRNA folding model is based on the optimization of parameters 1 and 2, and the cofold model is based on the optimization of parameters 1, 2, and 3. We show that ETIP outperformed the simpler models in terms of the correlation with the Z-scored CAI. The optimal correlations and the distance from the null model of all the three energy models (mRNA fold, cofold, ETIP) can be seen in FIGS. 12 and 13.

In this study three energy-based models were conducted that predict gene expressions levels in different chloroplasts' orthologues groups. By every energy-based model we were able to investigate the local folding of the mRNA and the local interaction between the mRNA and the 16S rRNA of the small ribosomal subunit for every group. We calculated the correlations between the energy values according to the optimized local parameters and the CAI scores for all the genes in the database, for every X, for the real and the null model. FIGS. 12A-C show the optimal correlations between the energy according to the model and CAI scores for all the genes in the database, for every X, and every model. For fold energy model the correlations are all positive, and co-fold and ETIP the correlations are all negative, although presented in the figure in absolute value, hence all the optimal correlations for all models are in the expected direction and are significant (above 0.62 with Pv<10\hat{ }(−324).). FIGS. 12D-12F present the distances between the optimal correlation of the real and the null models, divided by the null model's STD. For every energy model, we looked for the optimized solution by the minimal X that received high correlation (preferably local maximum) and high distance between the real and null model. For fold, cofold, and ETIP the optimal solutions were received for X=11, X=7, X=5 respectively. FIG. 13 presents the optimal correlation received for every energy model, with a total of approximately 16,000 points.

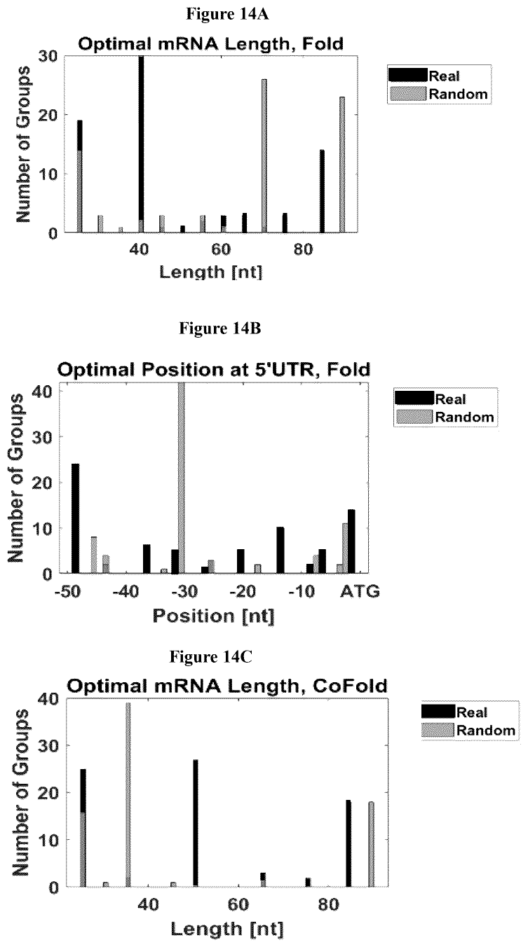

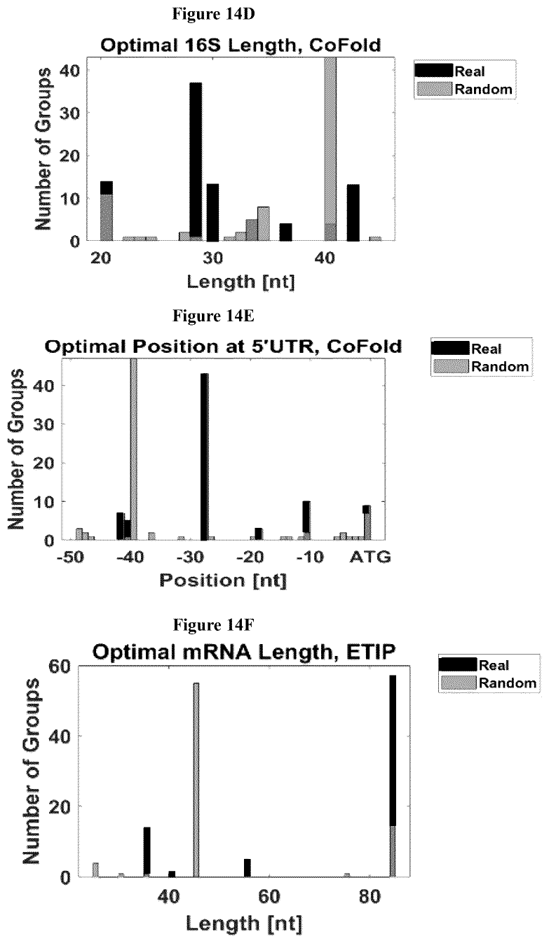

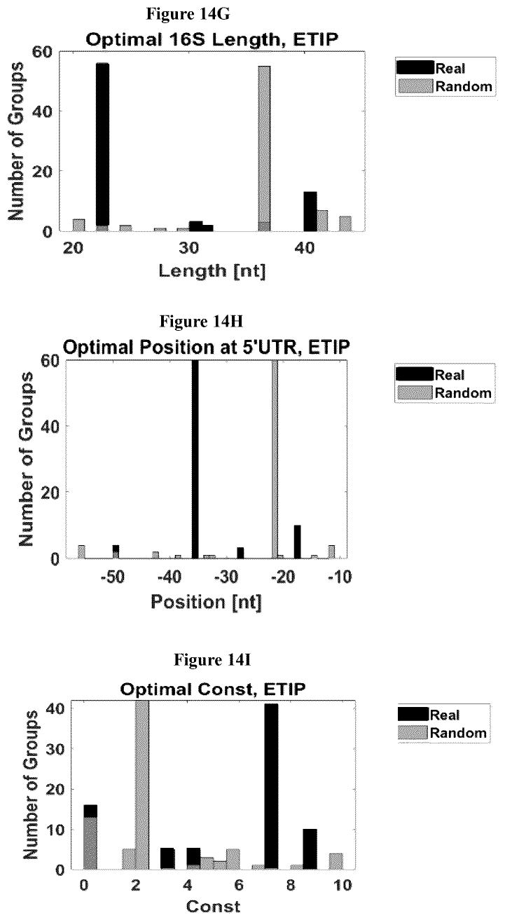

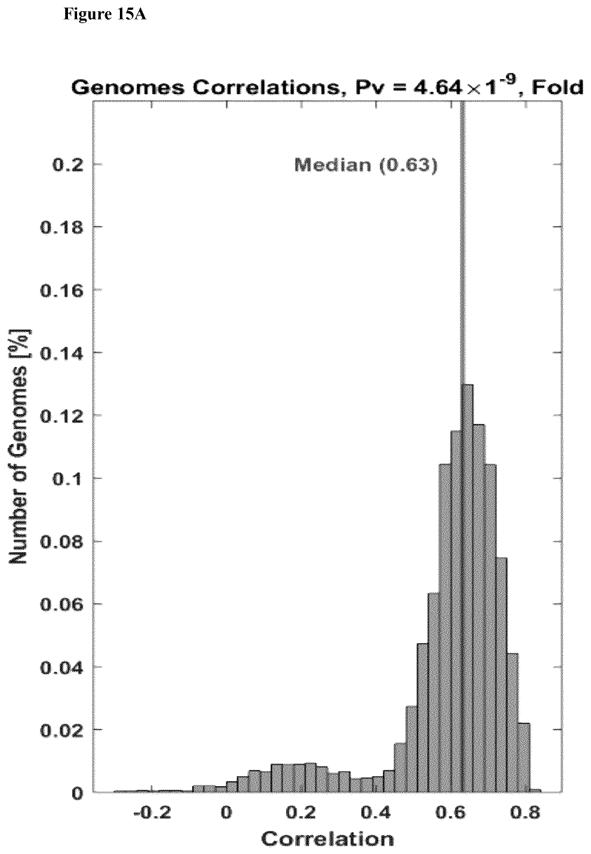

The optimized parameters for every ortholog group were taken from the optimal correlation of X=11, X=7, X=5 for fold, cofold, and ETIP models respectively. The parameters are shown in FIG. 14. It can be observed that all the majority parameters values of the real models differ from the null model which gives confidence that the results are meaningful. In addition, every model has parameters that optimize the prediction for a majority of groups which mostly varies between the different models.

### Optimization Process for the Energy-Based Model:

The optimization process was conducted by hill-climbing which is an optimization algorithm that makes local steps that improve the objective function until reaching optimization. We randomly divided all the genes in the database into 50% train and 50% test sets such that every set included an equal number of genes from every ortholog group. The objective function, in this case, is the Spearman correlation between the energies' values and the Z-scored CAI of the genes in the train set, which is expected to be high negative, since as negative the ETIP energy result is, the higher the probability of the hybridization to occur resulting translation initiation more efficient, therefore higher PA values and CAI scores.

In the optimization process, the aim is to choose the values of the local structure's four energy parameters that optimize the correlation. Every parameter has a set of values that can be checked and can be selected in the process:
1) mRNA window length, 25-90 nt in steps of 5 nt.
2) 16S rRNA window length from its 3′ end, 20-45 nt in steps of 1 nt.
3) Start position of the mRNA window on the 5′UTR of the gene, 50-0 nt upstream of the start codon, 0 means the start codon itself, in steps of 1 nt.
4) Constant of ETIP, 0-10 in steps of 0.5.
When the mRNA window size (parameter 1) is bigger than the start position (parameter 3), the sequence of the mRNA's local structure includes the start codon of the gene.

As already explained, the solution of the process will be such that every group has a set of four parameters, one for every parameter type; such a set is considered as the optimized parameters set for the group.

In addition, constrains were added to the hill-climbing algorithm such that the chosen parameters are not from the entire range described above but are from a limited sub-set in size of X that was sampled from this range. We added these constraints to simplify the model and to reduce overfitting; these constraints are also based on the assumption that there is a finite (relatively small) number of regulation strategies in chloroplasts that tend to appear in many genes.

The process was performed for X=3,5,7,9,11,13 and we also checked the option that the parameters' values set were not limited at all (i.e., each parameter can be selected from the entire range mentioned above). Eventually, the results were validated by the test set and were compared to the null model.

### Null Model for Energy-Based Model:

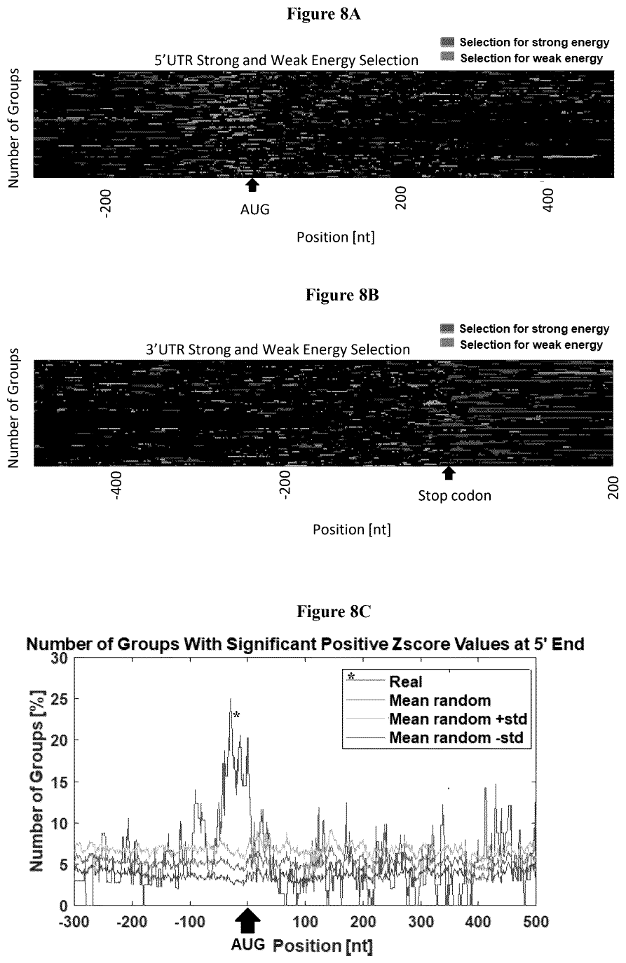

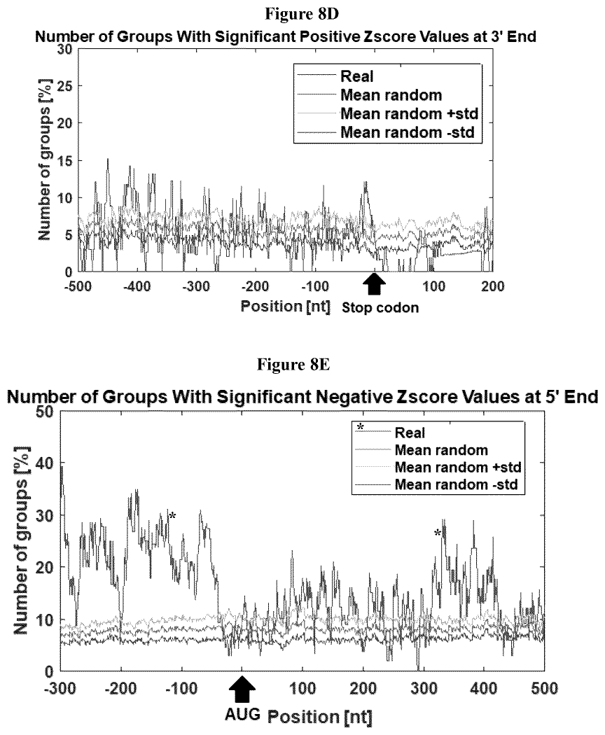

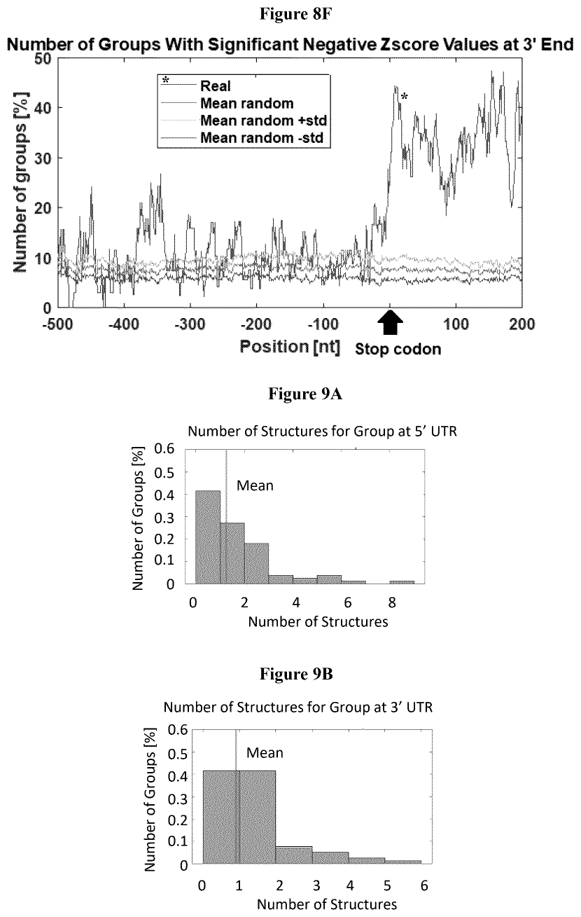

Two types of null models were conducted. The first one is based on shuffling the z-scored related to the CAI values between the genes of an ortholog group, this operation maintains all the fundamental properties of the real ortholog group (e.g., the amino acid sequence, codon usage, GC content, and evolutionary conservation). The second null model includes a more global shuffling: the z-scored related to the CAI between all the genes in the database was shuffled. The results of the first, less global, null model appears in FIGS. 12A-F, and the results of the second one are presented in FIGS. 8A-F.

A Regressor for Predicting the PA Value and Ribosomal Profiling Values of Chlamydomonas reinhardtii's Genes:

To show that the ETIP model adds predictive information over the CAI values, a regressor was inferred in order to predict the PA values and the ribosomal profiling values of Chlamydomonas reinhardtii's genes based on the combinations of CAI scores and the ETIP values; its performances were compared to prediction based only on CAI. The predictor was based on ranked values of all the variables, and it was evaluated by computing Spearman correlation. In addition, computed partial correlations to show that ETIP has significant correlation with PA values and the ribosomal profiling when controlling for the CAI values.

### P-Values:

All empiric P-values in this study were calculated as the fraction of null model randomization with higher/lower value than the real model. P-values lower than 0.05 re considered significant.

P-Value of Highly Expressed Genes in the Non-Typical Groups, and Lowly Expressed Genes in the Typical Groups, for Genes of Chlamydomonas reinhardtii:

In order to find out if the Chlamydomonas reinhardtii's genes that are in the typical/non-typical groups tend to be highly or lowly expressed, the average PA of the genes in the typical/non-typical groups were compared via a permutation test to the average PA of 100 sampled Chlamydomonas reinhardtii's gene with similar size to the typical/non-typical groups.

aSD-SD Interaction PSSM in Chlamydomonas reinhardtii:

In order to receive the positions where the aSD-SD interaction is most likely to appear in the 5′UTR of the mRNA in Chlamydomonas reinhardtii's genes, the hybridization energy between the mRNA and the aSD of prokaryotes (‘UCCUCC’) was calculated with ViennaRNA RNAcofold algorithm, for a 6 nt length sequence of the mRNA in a sliding window of 1 nt until the start codon. As a result, the position with the lowest interaction score (cofold energy) was received for every gene. In order to find out if the Chlamydomonas reinhardtii's genes that are in the typical/non-typical groups tend to have strong/weak aSD-SD interaction, the average interaction score of the genes in the typical/non-typical groups were compared via a permutation test to the average interaction score of 100 sampled Chlamydomonas reinhardtii's gene with similar size to the typical/non-typical groups.

Significant Strong/Weak Folding Selection in Different Positions Upon mRNA:

In order to investigate the positions with significant strong/weak folding selection upon the mRNA of genes in an ortholog group, folding energies of the mRNA were calculated by RNAfold of ViennaRNA package, with a sliding window of 1 nt in size of 39 nt such that the energies are calculated for every position at the mRNA, divided into the 5′UTR (positions at the 5′UTR, from the start of 5′UTR until the start codon; the last window includes 38 nt of the ORF) and 3′UTR (positions at the 3′UTR, from the stop codon until the end of the 3′UTR; the first window include 38 nt of the ORF).

For every group, the average folding energy values were calculated for every position of the mRNA, and Z-score and empiric P-values were calculated for every position with 50 null models, with an alignment to the start codon or the stop codon.

Z-score was calculated according to equation (3) and P-values were estimated empirically based on the null model mentioned above. A significant P-value was considered lower than 0.05.

In order to define the threshold for an unusual Z-score value in a certain position, we compared it to the position surrounding it via the following procedure:
the difference between the Z-score of the position and the average Z-scores of the surrounding of 100 nt (100 nt to the left, and 100 nt to the right) was calculated according to equation (5):

$$Diff = {Zscore}_{i} - \frac{( {\sum }_{i - 100}^{i + 100} {Zscore}_{j} )}{200} \qquad ( 5 )$$

The threshold of this measure (Diff) was computed based on the distribution of values of this measure over for orthologous groups generated by the null model; the top and bottom 5 percentile (corresponding to the Diff value of −1.5 and 1.5) were used as the significant Z-score threshold.
   Detecting the Conserved Functional Secondary Structures of the mRNA:

It is known that mRNA molecule tends to include local functional structures that, among others, can regulate gene expression. We expect that functional structures will be relatively conserved in comparison to non-functional ones. In order to detect the functional consensus secondary structure in the different orthologous groups we performed the following steps: first, the MSA of the mRNAs (nucleotide 3′/5′ UTR MSA and amino acid ORF MSA) was computed by Clustal Omega. Next, the folding energies and Z-score were calculated similar to the previous section on the MSAs, for positions at the 5′UTR (in this case the folding energies for every position are aligned to the start codon) and for positions at the 3′UTR (in this case the folding energies for every position are aligned to the stop codon).

Positions on the mRNA with significant negative Z-score compared to the surrounding Z-score values (which are considered as significant strong energy) were entered into RNAalifold—a ViennaRNA tool which predicts a consensus secondary structure of a set of aligned sequences; this tool finds a structure in this region that is conserved in all the genes in the MSA and reaches the minimum free energy using dynamic programming. The output of the RNAalifold algorithm is the consensus secondary structure, its free energy, the predicted frequency of the structure, and the predicted diversity of the structure. The structures are divided into two regions in the gene: the 5′ end of the transcript (the 5′UTR and the beginning of the ORF) and the 3′ end (the 3′UTR and the end of the ORF).

### Null Model for Detecting the Conserved Functional Secondary Structures:

In this subsection we describe how we computed a null model that maintains various fundamental properties of the mRNA MSAs such as the GC content, codon distribution, the encoded proteins, and the sequence distances (and that are related to the evolutionary distances among the sequences in the MSA) induced by the MSA. ORF MSA randomization included swapping the codons (while ignoring positions with indels) of the same AAs between two columns of the MSA that are similar in more than 95% of the AAs, and while considering only columns that have no more than 15% indels. UTR MSA randomization included swapping the nucleotides between two columns that have no more than 20% indels. We performed in each case 10n columns swapping, when n is the length of the MSA (in AAs for the ORF MSA, and in nts for the UTR MSA).

Energy-Based Models' Correlations with CAI Scores for Every Chloroplast's Genome:

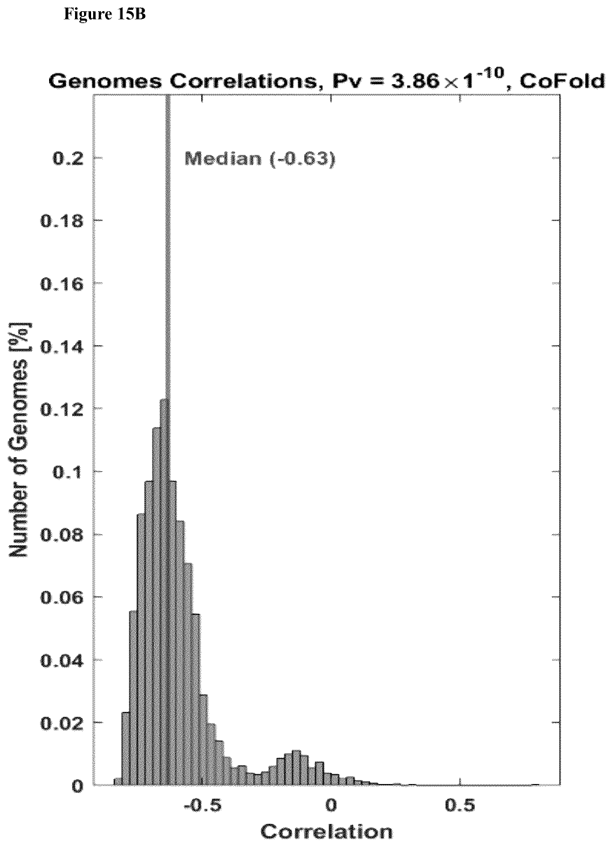

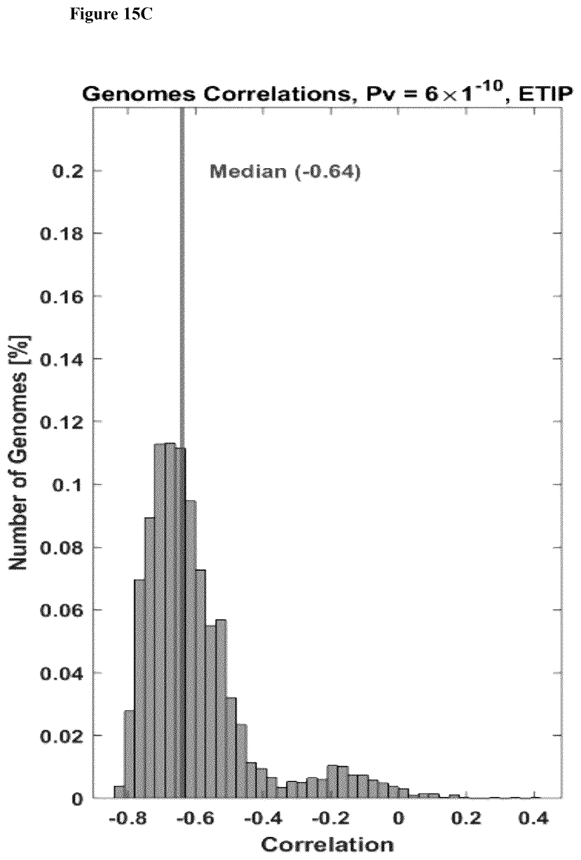

We calculated the correlations for every chloroplast's genome separately by calculating the energy for every gene of a certain chloroplast according to the optimized parameters of the ortholog group it belongs to. FIG. 15 shows the correlations for all chloroplast's genomes in the database, for every energy model. According to this figure it is demonstrated that the correlations are in the direction as expected, the median correlation of all the genomes for fold, cofold, and ETIP models are r=0.63, r=−0.63, and r=−0.64 respectively, with P-values of the genome with the median correlation of Pval=0.027, Pval=0.031 and Pval=0.026 respectively according to FIG. 15A-C.

### Source Code:

All the code generated in this analysis appears in cs.tau.ac.il/˜tamirtul/Chloroplas Trans/.

### Example 1: Experimental Overview

The flow diagram of the study is described in FIG. 1 (all details are in the Materials and methods section). The analyzed database includes the genome of 4,306 chloroplasts from various species; each chloroplast includes 74 genes on average so that 318,315 genes were analyzed in total (FIG. 1, section A). The study is divided into two main parts: the first one includes the development of an energy-based model for translation initiation prediction, and the second part includes the development of a large-scale database with predicted local functional mRNA structure.

The genes in the database were divided into orthologous groups to find the translation regulation characteristics for different gene products. An energy-based model was inferred using the codon adaptation index (CAI) as a proxy for expression levels (see Materials and methods); for every gene in the database, the CAI was computed and was normalized to be comparable to the CAI of genes from other chloroplasts (FIG. 1, section B). The energy values in the model were based on the local minimum free energy of a sequence. The local rRNA-mRNA hybridizations (FIG. 1, section C) and local mRNA folding (FIG. 1, section D) were calculated for every gene in the database. Employing these three steps (B-D) an Energy-based Translation Initiation Predictor (ETIP) was conducted (FIG. 1, section H). The local mRNA folding was computed for every gene in every ortholog group (FIG. 1, section D) and compared to the local mRNA folding generated based on a null model (FIG. 1, section F). Selection for strong mRNA energy was detected (FIG. 1, section G) in the multiple sequence alignment (MSA) of every ortholog group (FIG. 1, section E). After inferring the positions in which there is selection for strong energy in the ortholog group of genes, the local common functional mRNA structures were predicted (FIG. 1, section I). Combining the energy-based gene expression prediction (FIG. 1, section H) and the local functional mRNA structures (FIG. 1, section I) provides a predictive mRNA-rRNA interaction biophysical model that can, among others, shed light on novel aspects of chloroplasts translation mechanism and regulation.

### Example 2: Folding and Co-Folding Between the 5′ UTR of the mRNA and the 16S Small Subunit of the Ribosome Predicts Expression Levels of Chloroplast Genes

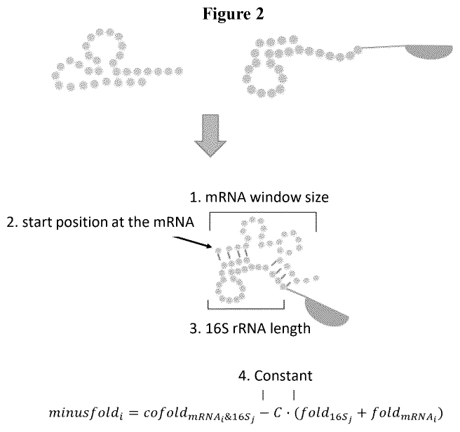

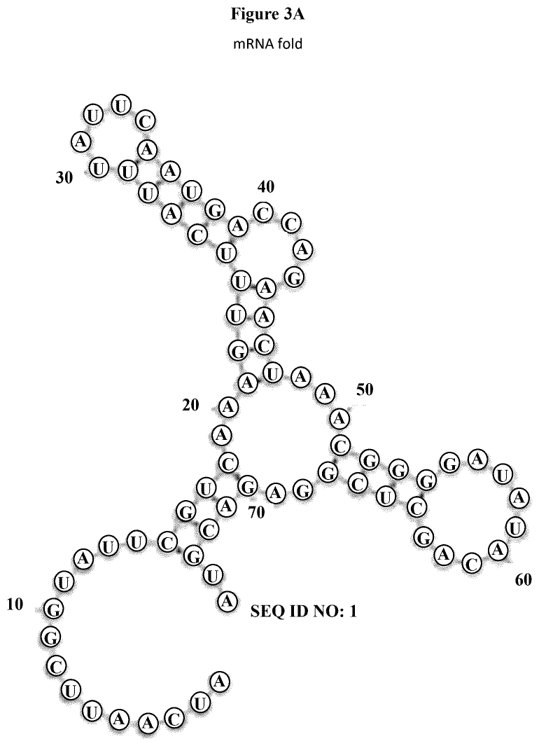

The purpose of this subsection is to conduct an energy-based model that will be able to evaluate and predict mRNA translation initiation efficiency. It is expected that there will be a high positive correlation between translation efficiency and protein abundance (PA) values, however, there are no measurements of PA for all the genes in the database, therefore normalized CAI scores, which are known to be highly correlated with PA, were used. This biophysical model is based on minimum free energy computations of the local mRNA-rRNA hybridization and folding, and the model compares the free energy in two states (as can be seen in FIG. 2): 1) before the 16S hybridizes to the mRNA when the mRNA and 16S exhibit self-folding structures; 2) after hybridization when the two sequences bind together and create a new co-folded structure. A higher decrease in the free energy in state 2) in comparison to state 1) is expected to be related to a more efficient initiation rate and higher probability of initiation (for further information see Materials and methods section 6 and 7).

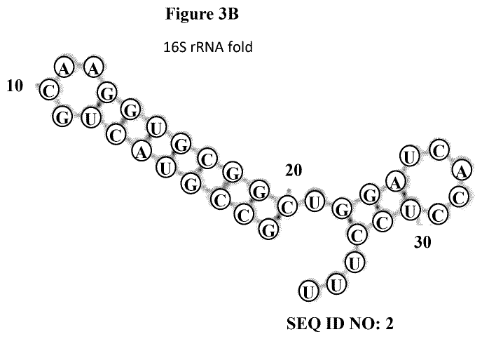

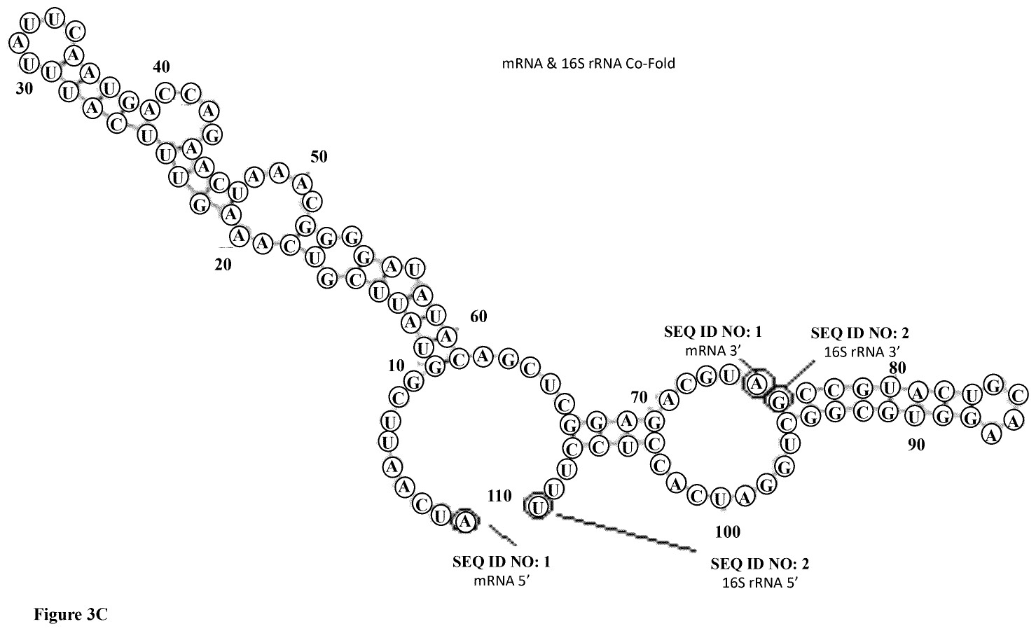

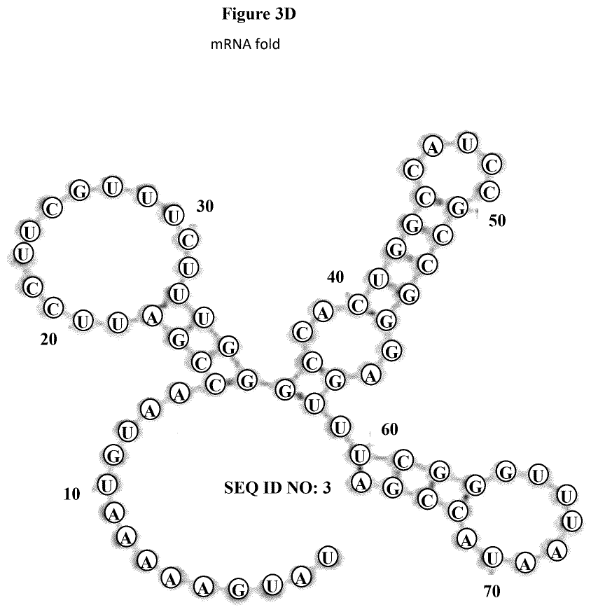

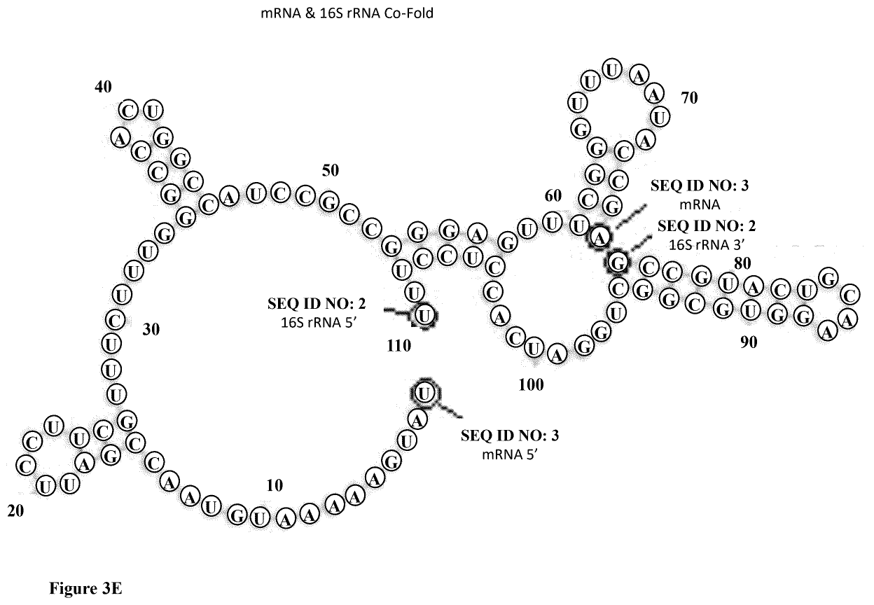

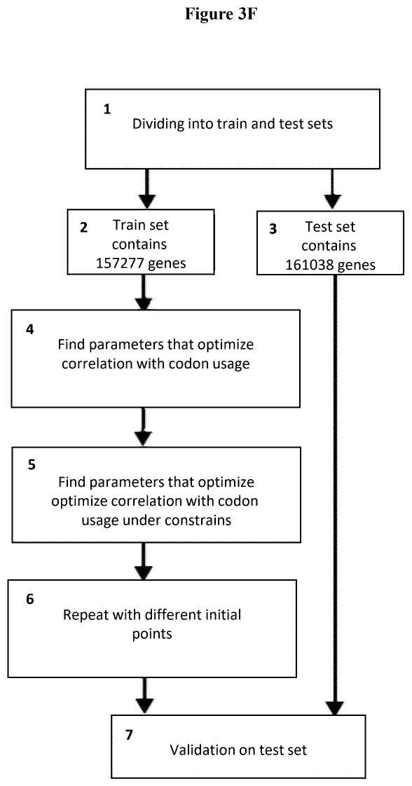

FIGS. 3A-F include examples of local self-folding structures of mRNA and 16S rRNA (FIG. 3B) and their co-folded structure (FIGS. 3C and 3E), for two different genes of Abelia Sanguinea's chloroplast (FIGS. 3A and 3D). Since translation efficiency is associated with higher protein levels, a negative Spearman correlation between the predictions of the model and PAs, and between the predictions and CAI scores is expected. Based on the free energy model, the typical properties of the local structures (four parameters) that compose the model were investigated (see FIG. 2): 1) the mRNA window length, 2) the position upon the 5′ UTR of the mRNA where the local structure starts, 3) the 16S rRNA sequence length from the 3′ edge, and 4) the ETIP constant that determines the subtraction of the self-folding from the co-folding energy as can be seen in FIG. 2.

The energy model relies on finding the optimized parameters out of a set of values such that the energy values calculated according to them will optimally predict the CAI scores. It is expected that the parameters that optimize the correlation will have meaning in terms of translation mechanism and will imply or reveal mRNA functional structures and properties of the mRNA-rRNA interactions that correspond to translation regulation and affect the translation efficiency.

According to the literature, different genes tend to have different translation regulations, therefore it is expected that different orthologous groups will have different optimal parameters of the energy-based model.

The different stages of the optimization process are described in a flow diagram in FIG. 3F, which describes with more details FIG. 1H of the project's global flow diagram.

As a first step, all the genes in the database were divided into train and test sets (FIG. 3F, section 1). Then a hill climbing optimization algorithm was applied on the train set to find the optimal energy parameters that predict the codon usage levels for every ortholog group (FIG. 3F, section 4). By assuming that there is a finite number of regulation strategies in chloroplasts constrains were added to the objective function, such that instead of examining all the values in every parameter's set, we took a subset of values in size of X (we checked different X values) that must include all the possible parameters of the model for all the orthologous groups. This way the model is simplified, and overfitting is reduced. For every X optimization the hill climbing was performed (FIG. 3F, section 5), with multiple different initiation points, by randomly selecting a new different subset of values to check (FIG. 3F, section 6). The case where the parameters weren't limited at all was also checked. Every initial point for every X reached an optimal correlation with optimal energy parameters for every group and then the correlations were validated with the test set and were compared to the null model (FIG. 3F, section 7). For further details, see Materials and methods section part 6.

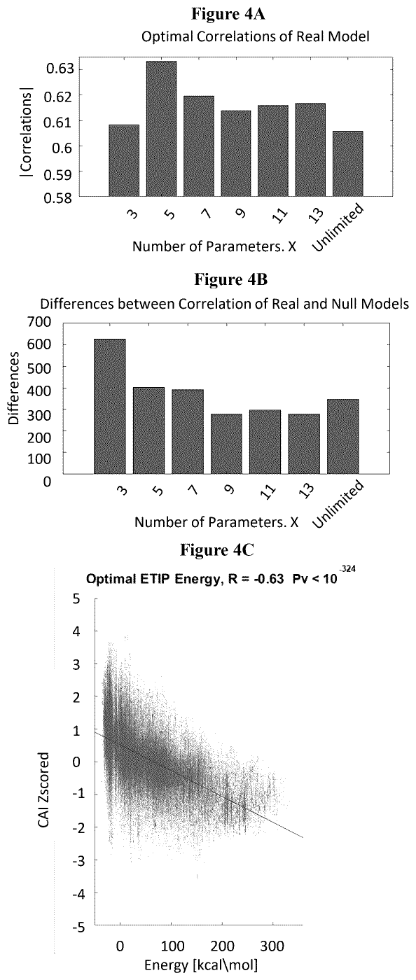

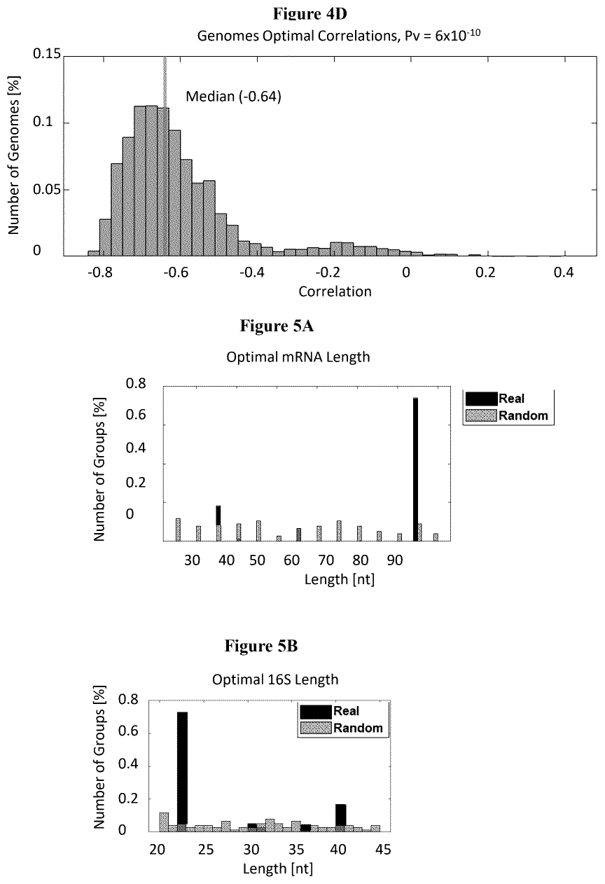

FIG. 4A shows the optimal correlation between the free energy model and CAI for every X. The correlations are all negative, although presented in the figure in absolute values, hence all the optimal correlations are in the expected direction and are significant: above 0.61 with P-value (Pv)<10\hat{ }(−324). The strongest correlation was obtained for X=5, which is also a local maximum between the correlations of X=3,7. FIG. 4B presents the distances between the optimal correlation of the real and the null models, divided by the null model's standard deviation (STD). The optimal solution from the minimal X that got high correlation in addition to high difference from the null model was selected. X=5 answers these conditions with a correlation of r=−0.63 with Pv<10\hat{ }(−324), and differs from the correlation of the null model by 400 STD. As elaborated in the Materials and Methods, we conducted two types of null models: in one of the permutations were less global than the other. All the results using the less global null model are presented hereinabove. FIGS. 12A-F also include the results related to all the three types of energy models we conducted. The scatter plot of the optimal correlation between the Z-scored CAI values and the energy values are shown in FIG. 4C with approximately 16,000 points. FIGS. 13A-C provide the dot plots of the optimal correlations for all the three energy models. The correlations of every chloroplast's genome in the database were calculated separately such that for every gene of a certain chloroplast the energy was calculated according to the optimized parameters of the ortholog group it belongs to. The genomes correlations can be seen in in FIGS. 4D and 15. The genome's optimal correlations are in the expected direction with a median correlation of r=−0.64, the Pv of the genome with the median correlation is Pv=6.10\hat{ }(−10); these results demonstrate that the energy model conducted in this study can be used as a gene expression predictor for every gene of every chloroplast's genome.

### Example 3: Different Genes Families in Chloroplasts have Different Translation Initiation Mechanisms which Mostly do not Rely on Shine-Dalgarno Interaction

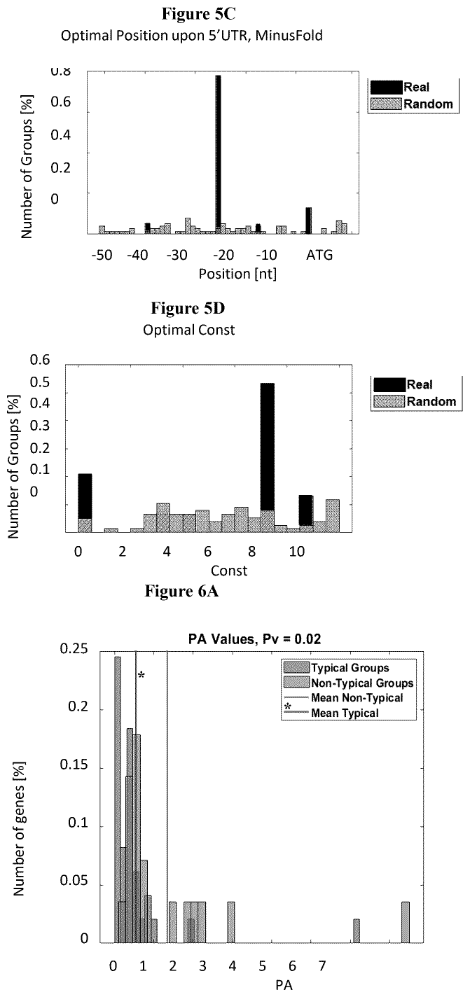

The optimized parameters for every ortholog group were taken from the optimal correlation of X=5. The parameters are shown in FIG. 5. First, it can be observed that the optimized parameters of the null model distribute uniformly, and all the parameters' values of the real model differ from the null model which gives confidence that the model is meaningful. As mentioned above, we also performed randomization which maintains various aspects of the real data; in this case the inferred values of the real model still significantly differ from the null model. The optimized parameters of the real and this null model for all three types of energy models can be seen in FIG. 14.

In addition, it can be seen that there are parameters that optimize the prediction for a majority of the orthologous groups. Groups that share the same optimized parameters that also belong to more than 10% of the groups are called “Typical groups”; the remaining groups are called “non-Typical groups”. For mRNA window length, the values that appear in more than 10% of the groups are: lengths of 85 nt (74%) and 35 nt (18%). As for the 16S window length, the typical parameter's values are: 22 nt (72%) and 41 nt (17%). The typical values of the parameter related to the position of the window at the 5′ UTR are: 26 nt (78%) and 7 nt (13%) upstream of the start codon; these three parameters have two peaks, one with ˜75% and the second one with ˜16% of the groups, that together sums up to ˜91%.

However, the constant parameter has three peaks, at 7 (53%), 0 (20%), and 9 (13%), which in total covers 87% of the groups. When considering all the typical values mentioned above, it is concluded that there are 49 (64%) groups that are considered as typical groups (i.e., groups that all their parameters are typical), and the rest (36%) are non-typical groups.

Later on, the sets of all four parameters mentioned above were studied. There are three sets of parameters that repeat in a high number of groups; the sets are used by 34%, 14%, and 12% of the typical groups respectively. The parameters of these sets are presented in Table 1, and according to it a 16S rRNA window lengths of 22 nt and positioned 26 nt upstream of the start codon are shared by all three sets (58% of the genes in the typical groups); in addition, the mRNA window length of 85 nt was present in 46% of the typical groups. In the case of the ETIP constant, the values 7 and 9 (which covers 44% of the typical groups) are close to each other and support the conjecture that the self-folding influences have a high effect on the translation efficiency; in addition, the constant 0, which indicates that the hybridization between the mRNA and the 16S is more important than the self-folding for the predictive power of the model mechanism, appears in 14% of the typical groups.

TABLE 1
Typical parameters sets.

| | | | Position at | |
|---|---|---|---|---|
| | mRNA | 16S | 5′ UTR | |
| Number | window | window | upstream | |
| of shared | length | length | start codon | ETIP |
| groups [%] | [nt] | [nt] | [nt] | Constant |
| 32 | 85 | 22 | 26 | 7 |
| 14 | 85 | 22 | 26 | 0 |
| 12 | 35 | 22 | 26 | 9 |

Therefore, it could be concluded that the first set in Table 1 includes the optimized parameters which probably have an important role in translation initiation regulation that affect the translation efficiency in most genes. The optimal parameters for every ortholog group can be seen in FIG. 14 and Appendix 1.

### Example 4: Genes of Chlamydomonas reinhardtii that Belong to the Typical Translation Initiation Regulation Groups do not Rely on Shine-Dalgarno Interaction

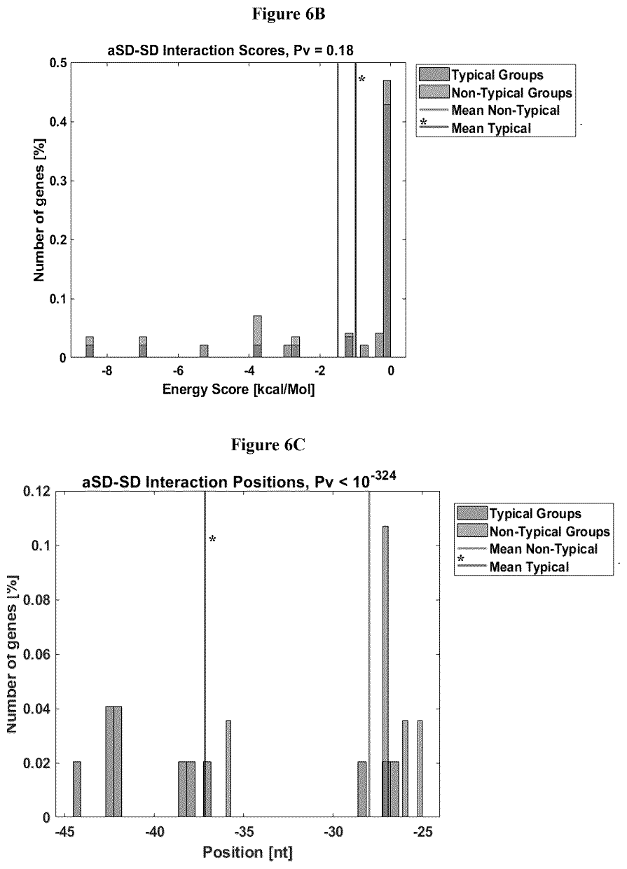

The genes related to the typical and non-typical groups were further investigated. According to Chlamydomonas reinhardtii's PA, the PA of the non-typical groups tend to be higher than the PA of other groups with Pv=0.02, in addition, the typical groups tend to be lowly expressed with Pv=0.03. The distribution of PA is presented in FIG. 6A.

At the next step, the average aSD-SD energy in Chlamydomonas reinhardtii for the typical and non-typical groups was calculated. It was found that the average energy of the non-typical groups is significantly stronger (−1.489) than the average energy of the typical groups (0.997), with Pv=0.18 (FIG. 6B). The average position of the SD sequence in the typical groups is 35-30 nt upstream of the start codon, whereas for the non-typical groups the average position of the SD sequence is 16-8 nt upstream of the start codon, in accordance with the typical position of SD in prokaryotes (FIG. 6C, Pv<10\hat{ }−324).

### Example 5: High Correlation of a Model which is Based on Codon Usage and the ETIP with Energy-Measurements of Protein Abundance and Ribosomal Profiling Values

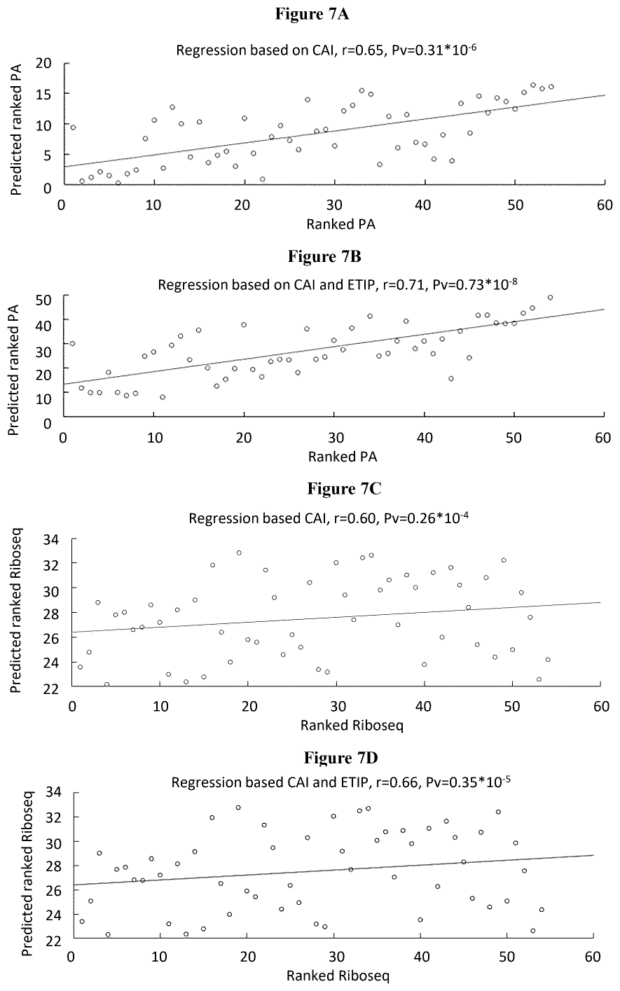

A regressor was conducted in order to predict the PA values of Chlamydomonas reinhardtii's genes, once with the CAI scores only and once with the CAI scores and the ETIP values. The Spearman correlation between the real PA values and the predicted ones was calculated. The correlation of the predicted PAs with a regression model based on the CAI scores is r=0.65 (Pv=3.10×10\hat{ }−7), whereas the correlation with the predicted PAs by the regression model based on the CAI scores and the ETIP values is r=0.71 (Pv=7.29×10\hat{ }−9). These results show that the energy-based model improves the PA predictions. The scatter plots of the predicted and real PAs are presented in FIG. 7. In order to examine the significance of additional information of the energy model towards the PA values, the partial correlation was calculated, and the result was r=−0.399 and Pv=0.003 which supports the conjecture that the energy-based model is useful for predicting PAs; moreover, with 95% confidence the coefficient of the energy in the regression is not zero, supporting the conjecture that it significantly improves the regression model.

Next, a similar process was conducted to predict the ribosomal profiling values of Chlamydomonas reinhardtii's genes. In this case the correlation of the predicted ribo-seq values with a regression model based only on the CAI scores is r=0.60 (Pv=2.6×10\hat{ }−5) whereas the correlation with the predicted ribo-seq values by the regression model based on the CAI scores and the ETIP values is r=0.66 (Pv=3.5×10\hat{ }−5). The partial correlation resulted with r=−0.33 and Pv=0.035, and the coefficient of the energy in the regression is not zero (also with 95% confidence). The scatter plots of the predicted and real PAs and ribo-seq values are presented in Error! Reference source not found.

### Example 6: Chloroplast Genes Tend to have Strong Structures Upstream of the Start Codon and Downstream of the Stop Codon

Next, functional local mRNA structures are inferred in different chloroplast genes. Their functionality can be reflected by interacting, among others, with the rRNA, protein factors, micro-RNAs, and by that can affect various gene expression mechanisms (for instance translation regulation, mRNA stability, mRNA transcription, mRNA transport, etc.). They can also affect translation by changing the distance between regulatory sequence motifs and the start codon or by interacting with regulatory sequence motifs. To this end, folding energies of the mRNA were calculated for every mRNA in the real and random groups and the selection for strong folding was determined by calculating Z-scores and empiric Pvs for every position in the mRNA. The regions of interest to examine the significance of the Z-score values were limited to the positions in the alignment in which more than 50% of the genes in the ortholog group contribute a nucleotide (i.e., the majority of the values in the position are not indels; see FIG. 11).

FIGS. 8A-B show the Pvs related to strong/weak folding energy in comparison to the null model for different positions along the mRNAs. FIGS. 8C-F present the number of groups that have significant Z-score values normalized by the number of groups with a nucleotide in the positions for the real groups and in comparison to the null model.

There are a few major findings related to this analysis: First, there are many groups with a significant negative Z-score downstream of the stop codon according to FIG. 8 Error! Reference source not found.B and 8F. Specifically, it can be seen that at 8-20 nt and 140-195 nt downstream of the stop codon there is a higher percent (˜45-48%) of groups with significant negative Z-scores which differs by more than 45 std from the average random Z-scores (Pv<10\hat{ }−324). This result suggests that the mRNA undergoes selection to be folded in the positions downstream of the stop codon, possibly to improve the efficiency of the termination by the release factors (RFs).

Second, as can be seen in FIGS. 8A and 8E, there is a significant negative Z-score at positions 70-50 nt, 185-170 nt and 280 nt upstream of the start codon. The number of groups with such scores is close to 40% in comparison to the null model, higher by more than 30 std from the average random Z-scores (Pv<10\hat{ }−324). This suggests that the mRNA tends to undergo selection to be strongly folded upstream of the start codon. It is possible that there are factors that react with the mRNA in these positions via these structures to promote initiation.

In addition, as can be seen in FIGS. 8A and 8C, it can be observed that the mRNA tends to be open at 30-1 nt upstream of the start codon; this may promote efficient recognition of the start codon by the initiation complex (as appears in many nuclear genomes). Specifically, ˜20-25% of the groups have this signal, which is also stronger by approximately 20 std than the average random Z-scores (Pv<10\hat{ }−324).

In FIG. 8D it can be seen that at the 3′ end there is no significant tendency for any position to be weakly folded in comparison to the null model.

### Example 7: Conserved Potentially Functional mRNA Structures at the Ends of the Coding Regions

mRNA molecules are populated with functional local structures that can affect gene expression regulation in various ways such as: 1) The binding of the RNA binding proteins (e.g., to the RNA loop); note that the existence of a structure can decrease the distance between the binding motif and the start codon and thus improve the translation efficiency. 2) Via base pairing the structures can prevent the interactions of RNA binding proteins with unwanted binding motifs. 3) The structure can improve the stability of the mRNA by blocking exonucleases. It is expected that functional structures will tend to be conserved throughout all the genes in the ortholog group and structures that are not conserved will probably not be functional. In this study, the functional consensus secondary structure for every ortholog group were detected. Such structures can be used for modeling and engineering gene expression in chloroplasts.

In order to predict the functional secondary structures, positions with significant strong energy folding were discovered by comparing the local self-folding energies of every position at the multiple sequence alignment (MSA) of the real mRNAs to the folding energies obtained by the null model in the same position. At the next step, the consensus secondary structures were detected for these positions on the MSA for every ortholog group by finding the structures based on a dynamic programming algorithm that searches for conserved minimum energy structure over the entire alignment (more details in the Materials and methods).

The information provided regarding every structure includes the consensus structure, the energy of the structure in kcal/mol, the expected frequency of the structure among large set of identical mRNAs and the expected structure's diversity related to the current position (i.e., how many different structures were expected in this position). The results of this Example include many consensus secondary structures related to various orthologous groups which are expected to be functional.

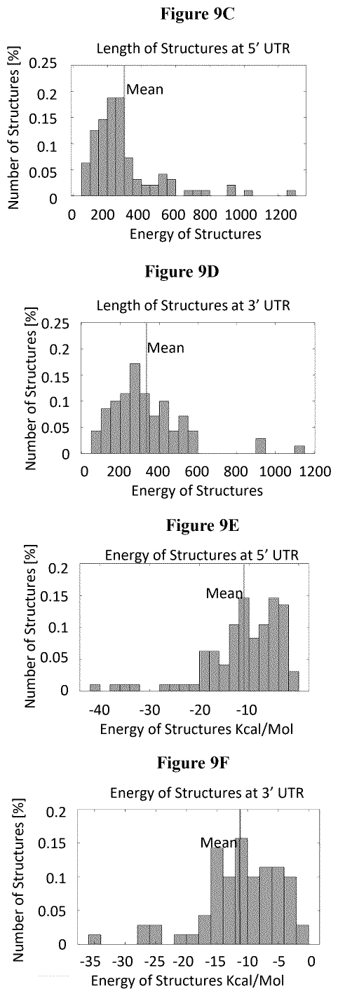

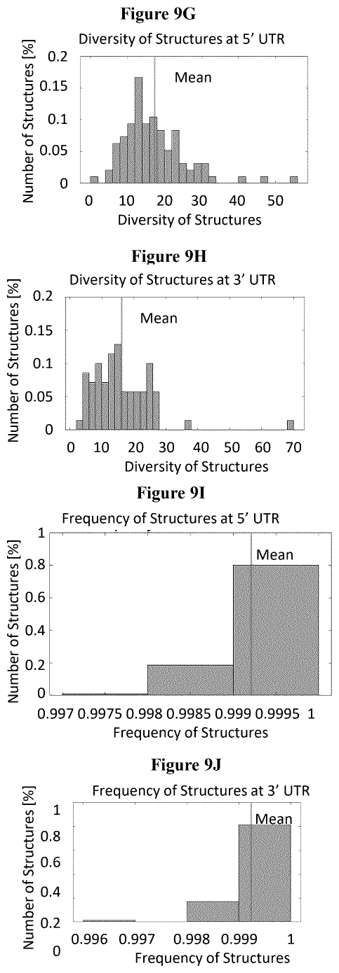

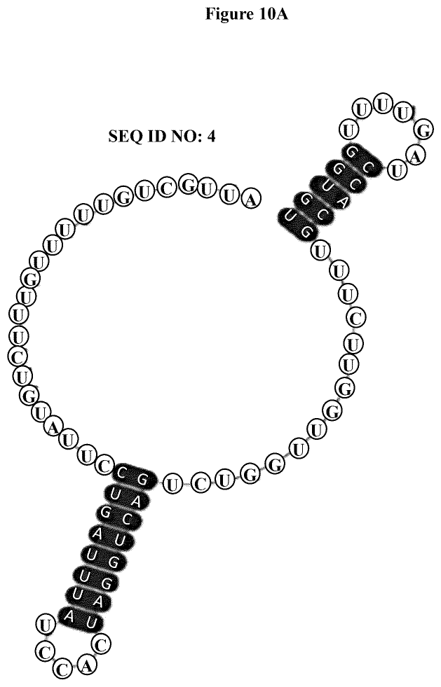

According to this analysis, some of the orthologous groups have more than one typical local structure, most of the groups have 0-2 structures in the 5′ UTR (i.e., structures that begin in the 5′ UTR and may also include part of the ORF) and 0-1 structures in the 3′ UTR (i.e., structures that end in the 3′ UTR but may begin in the ORF), as can be seen in FIGS. 9A and 9B. The lengths of the structures in the 5′ UTR are between 50 and 350 nt while most of them have a length of ˜100 or ˜200 nt (FIG. 9C), and most of the structures in the 3′ UTR are in the range of 200-450 nt (FIG. 9D). The lengths of the structures and their geometry may be related to the lengths and properties of the factor/s that bind to these structures which contributes to translation initiation or termination.

The frequencies of the structures both at the 5′ UTR and 3′ UTR are very high and close to 100% (FIG. 9E-F; i.e., almost 100% of the mRNA copies are expected to have the predicted structures). The diversities of the structures at the 5′ UTR mostly ranges between 8-24 (FIG. 9G) and similarly, at the 3′ UTR the diversities range between 1-28 (FIG. 9H). Low diversity means that the molecule has fewer options for local probable structures to fold into, therefore the probability of the molecule being folded in the detected structure is higher. The energies of the structures at the 5′ UTR and 3′ UTR ranges between-20 to −2 kcal/mol (FIG. 9I-J).

### Example 8: a Database of Novel Conserved Secondary Structures in Chloroplasts

Herein is reported a database with 96 conserved structures in the 5′ UTR and 70 conserved structures in the 3′UTR that were detected. All the structures are provided in Ezra and Tuller, “Modeling the effect of rRNA-mRNA interactions and mRNA folding on mRNA translation in chloroplasts”, Comput. Struct. Biotechnol. J., 2022 May 18; 20:2521-2538, in Supplementary Data 3, herein incorporated by reference in its entirety and in US Provisional Application U.S. 63/337,113. Information regarding the structure's position on the mRNA, length, and energy are provided in Table 2.

TABLE 2
Information regarding the 166 structures of the invention.
Structure numbers correspond to those provided in Table 3.

| | | | Length | Energy |
|---|---|---|---|---|
| # | Gene | Position | (nt) | (kcal/mol) |
| 1 | ATP synthase CF1 subunit alpha | 955 nt DS stop codon | 152 | −11.2 |
| 2 | ATP synthase CF1 subunit alpha | 1153 nt DS stop codon | 307 | −14.4 |
| 3 | ATP synthase CF1 subunit alpha | 1504 nt DS stop codon | 192 | −15.5 |
| 4 | ATP synthase CF1 subunit beta | 573 nt US start codon | 655 | −14.2 |
| 5 | ATP synthase CF1 subunit beta | 82 nt US stop codon | 85 | −10.9 |
| 6 | ATP synthase CF1 subunit beta | 126 nt DS stop codon | 17 | −17.5 |
| 7 | ATP synthase CF1 subunit beta | 251 nt DS stop codon | 98 | −11.6 |
| 8 | ATP synthase CF1 subunit beta | 305 nt DS stop codon | 91 | −13.8 |
| 9 | ATP synthase CF1 subunit epsilon | 1336 nt US start codon | 90 | −18 |
| 10 | ATP synthase CF1 subunit epsilon | 1271 nt US start codon | 131 | −26.6 |
| 11 | ATP synthase CF1 subunit epsilon | 981 nt US start codon | 115 | −16.5 |
| 12 | ATP synthase CF1 subunit epsilon | 846 nt US start codon | 132 | −25.1 |
| 13 | ATP synthase CF1 subunit epsilon | 716 nt US start codon | 137 | −35.4 |
| 14 | ATP synthase CF1 subunit epsilon | 482 nt US start codon | 76 | −19.3 |
| 15 | ATP synthase CF1 subunit epsilon | 326 nt US start codon | 92 | −19.4 |
| 16 | ATP synthase CF1 subunit epsilon | 233 nt US start codon | 79 | −10.3 |
| 17 | ATP synthase CF1 subunit epsilon | 37 nt US start codon | 122 | −10.7 |
| 18 | ATP synthase CF1 subunit epsilon | 53 nt US stop codon | 429 | −27.6 |
| 19 | ATP synthase CF1 subunit epsilon | 284 nt DS stop codon | 335 | −16.6 |
| 20 | ATP synthase CF1 subunit epsilon | 851 nt DS stop codon | 227 | −4.7 |
| 21 | ATP synthase CF1 subunit epsilon | 1062 nt DS stop codon | 145 | −9.6 |
| 22 | ATP synthase CF1 subunit epsilon | 1143 nt DS stop codon | 111 | −10.2 |
| 23 | ATP synthase CF1 subunit epsilon | 1232 nt DS stop codon | 161 | −9.4 |
| 24 | ATP synthase CF0 subunit I | 406 nt US start codon | 227 | −1.4 |
| 25 | ATP synthase CF0 subunit I | 207 nt US start codon | 262 | −7.5 |
| 26 | ATP synthase CF0 subunit III | 382 nt US start codon | 176 | −3.3 |
| 27 | ATP synthase CF0 subunit III | 310 nt US start codon | 36 | −11.6 |
| 28 | ATP synthase CF0 subunit IV | 262 nt US start codon | 286 | −7.5 |
| 29 | ATP synthase CF0 subunit IV | 177 nt US start codon | 295 | −10.6 |
| 30 | ATP synthase CF0 subunit IV | 219 nt DS stop codon | 234 | −4.6 |
| 31 | Cytochrome c biogenesis protein CCSA | 744 nt US start codon | 123 | −7.9 |
| 32 | Cytochrome c biogenesis protein CCSA | 526 nt US start codon | 575 | −2 |
| 33 | Cytochrome c biogenesis protein CCSA | 38 nt US stop codon | 312 | −3.4 |
| 34 | Envelope membrane protein | 1 nt US stop codon | 284 | −11 |
| 35 | CLP protease ATP binding subunit | 629 nt US start codon | 782 | −18.8 |
| 36 | CLP protease ATP binding subunit | 714 nt DS stop codon | 157 | −13.3 |
| 37 | CLP protease ATP binding subunit | 947 nt DS stop codon | 100 | −15.1 |
| 38 | intron maturase K | 1600 nt US start codon | 229 | −2.8 |
| 39 | intron maturase K | 1389 nt US start codon | 154 | −3.4 |
| 40 | intron maturase K | 1217 nt US start codon | 1296 | −37.6 |
| 41 | intron maturase K | 36 nt DS stop codon | 487 | −4.3 |
| 42 | Translation initiation factor IF-1 | 244 nt US start codon | 288 | −18.6 |
| 43 | Translation initiation factor IF-1 | 70 nt US stop codon | 541 | −18.7 |
| 44 | NADH-plastoquinone oxi-doreductase subunit 2 | 286 nt US start codon | 318 | −20 |
| 45 | NADH-plastoquinone oxi-doreductase subunit 2 | 80 nt US stop codon | 465 | −7.7 |
| 46 | NADH-plastoquinone oxi-doreductase subunit 2 | 35 nt US stop codon | 514 | −1.9 |
| 47 | NADH-plastoquinone oxi-doreductase subunit 3 | 1255 nt US start codon | 281 | −6 |
| 48 | NADH-plastoquinone oxi-doreductase subunit 3 | 976 nt US start codon | 1027 | −41.4 |
| 49 | NADH-plastoquinone oxi-doreductase subunit 3 | 76 nt US stop codon | 165 | −12.4 |
| 50 | NADH-plastoquinone oxi-doreductase subunit 4 | 29 nt US stop codon | 365 | −8 |
| 51 | NADH-plastoquinone oxi-doreductase subunit 4L | 615 nt US start codon | 157 | −10.7 |
| 52 | NADH-plastoquinone oxi-doreductase subunit 4L | 568 nt US start codon | 595 | −8.5 |
| 53 | NADH-plastoquinone oxi-doreductase subunit 4L | 40 nt US stop codon | 352 | −9.8 |
| 54 | NADH-plastoquinone oxi-doreductase subunit 5 | 501 nt US start codon | 535 | −13.5 |
| 55 | NADH-plastoquinone oxi-doreductase subunit 5 | 113 nt US stop codon | 115 | −2.7 |
| 56 | NADH-plastoquinone oxi-doreductase subunit 6 | 254 nt US start codon | 259 | −3.2 |
| 57 | NADH-plastoquinone oxi-doreductase subunit 7 | 108 nt US start codon | 237 | −22.7 |
| 58 | NADH-plastoquinone oxi-doreductase subunit I | 276 nt US start codon | 292 | −6.9 |
| 59 | NADH-plastoquinone oxi-doreductase subunit I | 4 nt DS stop codon | 425 | −9.1 |
| 60 | NADH-plastoquinone oxi-doreductase subunit K | 109 nt US stop codon | 294 | −6 |
| 61 | Cytochrome f | 411 nt US start codon | 435 | −6.1 |
| 62 | Cytochrome f | 366 nt US start codon | 447 | −8.3 |
| 63 | Cytochrome f | 42 nt US stop codon | 516 | −14.3 |
| 64 | Cytochrome f | 247 nt DS stop codon | 330 | −13.48 |
| 65 | Cytochrome f | 412 nt DS stop codon | 251 | −15.18 |
| 66 | Cytochrome f | 474 nt DS stop codon | 252 | −11.1 |
| 67 | Cytochrome b6 | 66 nt US stop codon | 365 | −14.6 |
| 68 | Cytochrome b6/f complex subunit 4 | 285 nt US start codon | 309 | −13.1 |
| 69 | Cytochrome b6/f complex subunit 4 | 190 nt US start codon | 255 | −21.3 |
| 70 | Cytochrome b6/f complex subunit 4 | 105 nt US stop codon | 475 | −25.8 |
| 71 | Cytochrome b6/f complex subunit 4 | 385 nt DS stop codon | 151 | −3.6 |
| 72 | Cytochrome b6/f complex subunit 5 | 237 nt US start codon | 236 | −8.9 |
| 73 | Cytochrome b6/f complex subunit 5 | 23 nt US stop codon | 417 | −8.9 |
| 74 | Cytochrome b6/f complex subunit 5 | 284 nt DS stop codon | 207 | −8.6 |
| 75 | Cytochrome b6/f complex subunit 5 | 647 nt DS stop codon | 243 | −7 |
| 76 | Cytochrome b6/f complex subunit 6 | 965 nt US start codon | 251 | −8.2 |
| 77 | Cytochrome b6/f complex subunit 6 | 738 nt US start codon | 252 | −5.9 |
| 78 | Cytochrome b6/f complex subunit 6 | 457 nt US start codon | 248 | −8 |
| 79 | Cytochrome b6/f complex subunit 6 | 342 nt US start codon | 179 | −2.7 |
| 80 | Cytochrome b6/f complex subunit 6 | 290 nt US start codon | 297 | −3.6 |
| 81 | Cytochrome b6/f complex subunit 6 | 6 nt US stop codon | 347 | −5.2 |
| 82 | Cytochrome b6/f complex subunit 8 | 1729 nt US start codon | 202 | −3.6 |
| 83 | Cytochrome b6/f complex subunit 8 | 1435 nt US start codon | 302 | −11.07 |
| 84 | Cytochrome b6/f complex subunit 8 | 1279 nt US start codon | 524 | −13.7 |
| 85 | Cytochrome b6/f complex subunit 8 | 1240 nt US start codon | 729 | −33.2 |
| 86 | Photosystem I P700 apoprotein A1 | 483 nt US start codon | 255 | −10.57 |
| 87 | Photosystem I P700 apoprotein A1 | 320 nt US start codon | 369 | −12.6 |
| 88 | Photosystem I P700 apoprotein A2 | 87 nt US start codon | 128 | −13.6 |
| 89 | Photosystem I P700 apoprotein A2 | 51 nt US stop codon | 235 | −12.8 |
| 90 | Photosystem I subunit VII (iron-sulfur center) | 360 nt US start codon | 365 | −3.6 |
| 91 | Photosystem I subunit VII (iron-sulfur center) | 212 nt US start codon | 326 | −8.5 |
| 92 | Photosystem I subunit VII (iron-sulfur center) | 50 nt US stop codon | 276 | −7.6 |
| 93 | Photosystem I reaction center subunit IX | 724 nt US start codon | 215 | −5.1 |
| 94 | Photosystem I reaction center subunit IX | 151 nt DS stop codon | 270 | −13.6 |
| 95 | Photosystem I reaction center subunit IX | 232 nt DS stop codon | 260 | −13.5 |
| 96 | Photosystem II reaction center protein D1 | 446 nt US start codon | 178 | −4.6 |
| 97 | Photosystem II reaction center protein D1 | 167 nt US start codon | 188 | −2.3 |
| 98 | Photosystem II reaction center protein D1 | 124 nt US start codon | 185 | −4.4 |
| 99 | Photosystem II reaction center protein D1 | 59 nt US stop codon | 268 | −14.6 |
| 100 | Photosystem II CP47 chlorophyll apoprotein | 516 nt US start codon | 332 | −2.1 |
| 101 | Photosystem II CP47 chlorophyll apoprotein | 33 nt US stop codon | 324 | −10.1 |
| 102 | Photosystem II CP43 chlorophyll apoprotein | 806 nt US start codon | 76 | −11.9 |
| 103 | Photosystem II CP43 chlorophyll apoprotein | 697 nt US start codon | 119 | −12.4 |
| 104 | Photosystem II CP43 chlorophyll apoprotein | 628 nt US start codon | 110 | −16.9 |
| 105 | Photosystem II CP43 chlorophyll apoprotein | 520 nt US start codon | 128 | −19.2 |
| 106 | Photosystem II CP43 chlorophyll apoprotein | 230 nt US start codon | 110 | −10.9 |
| 107 | Photosystem II CP43 chlorophyll apoprotein | 125 nt US start codon | 180 | −13.6 |
| 108 | Photosystem II CP43 chlorophyll apoprotein | 65 nt US stop codon | 590 | −16 |
| 109 | Photosystem II cytochrome b559 alpha subunit | 542 nt US start codon | 557 | −5.5 |
| 110 | Photosystem II protein K | 1076 nt US start codon | 258 | −4.4 |
| 111 | Photosystem II protein K | 890 nt US start codon | 184 | −14.2 |
| 112 | Photosystem II protein K | 796 nt US start codon | 206 | −14.2 |
| 113 | Photosystem II protein K | 688 nt US start codon | 238 | −3 |
| 114 | Photosystem II protein K | 184 nt DS stop codon | 219 | −4.5 |
| 115 | Photosystem II protein N | 201 nt US start codon | 247 | −13.9 |
| 116 | Photosystem II subunit PsbZ | 790 nt US start codon | 228 | −16.2 |
| 117 | Photosystem II subunit PsbZ | 701 nt US start codon | 243 | −8.1 |
| 118 | Photosystem II subunit PsbZ | 522 nt US start codon | 205 | −4 |
| 119 | Photosystem II subunit PsbZ | 406 nt US start codon | 240 | −14.7 |
| 120 | Photosystem II subunit PsbZ | 225 nt US start codon | 263 | −13.6 |
| 121 | Photosystem II subunit PsbZ | 107 nt US stop codon | 561 | −10.9 |
| 122 | Photosystem II subunit PsbZ | 318 nt DS stop codon | 521 | −10.87 |
| 123 | Photosystem II subunit PsbZ | 492 nt DS stop codon | 424 | −6.37 |
| 124 | RuBisCo large subunit | 689 nt US start codon | 136 | −4.7 |
| 125 | RuBisCo large subunit | 433 nt US start codon | 253 | −4.5 |
| 126 | RuBisCo large subunit | 195 nt US start codon | 264 | −9.6 |
| 127 | RuBisCo large subunit | 41 nt US stop codon | 345 | −7.9 |
| 128 | RuBisCo large subunit | 1 nt US stop codon | 406 | −11.1 |
| 129 | Ribosomal protein L14 | 75 nt US stop codon | 436 | −21.6 |
| 130 | Ribosomal protein L2 | 49 nt US stop codon | 935 | −3 |
| 131 | Ribosomal protein L20 | 1278 nt US start codon | 194 | −5.5 |
| 132 | Ribosomal protein L20 | 1218 nt US start codon | 233 | −10.1 |
| 133 | Ribosomal protein L20 | 880 nt US start codon | 94 | −10.3 |
| 134 | Ribosomal protein L20 | 694 nt US start codon | 180 | −5.9 |
| 135 | Ribosomal protein L20 | 484 nt US start codon | 501 | −11.5 |
| 136 | Ribosomal protein L22 | 199 nt US start codon | 312 | −13.6 |
| 137 | Ribosomal protein L23 | 496 nt US start codon | 506 | −16.2 |
| 138 | Ribosomal protein L23 | 399 nt US start codon | 475 | −5.9 |
| 139 | Ribosomal protein L32 | 179 nt US start codon | 185 | −6.8 |
| 140 | Ribosomal protein L32 | 78 nt US start codon | 190 | −6.4 |
| 141 | Ribosomal protein L32 | 68 nt US stop codon | 1114 | −35.8 |
| 142 | Ribosomal protein L33 | 418 nt US start codon | 181 | −5.3 |
| 143 | Ribosomal protein L33 | 304 nt US start codon | 306 | −8.9 |
| 144 | Ribosomal protein L36 | 38 nt US stop codon | 252 | −2.5 |
| 145 | RNA polymerase α subunit (PEP) | 156 nt US start codon | 240 | −17 |
| 146 | RNA polymerase α subunit (PEP) | 57 nt US stop codon | 575 | −9.6 |
| 147 | Ribosomal protein S11 | 197 nt US start codon | 247 | −7.2 |
| 148 | Ribosomal protein S11 | 67 nt US stop codon | 218 | −11.3 |
| 149 | Ribosomal protein S14 | 188 nt US start codon | 205 | −3.2 |
| 150 | Ribosomal protein S14 | 67 nt US stop codon | 355 | −25 |
| 151 | Ribosomal protein S14 | 253 nt DS stop codon | 268 | −27 |
| 152 | Ribosomal protein S14 | 564 nt DS stop codon | 147 | −14.4 |
| 153 | Ribosomal protein S15 | 38 nt US stop codon | 532 | −15.5 |
| 154 | Ribosomal protein S16 | 1369 nt DS stop codon | 189 | −4 |
| 155 | Ribosomal protein S18 | 900 nt US start codon | 927 | −10.4 |
| 156 | Ribosomal protein S18 | 834 nt US start codon | 914 | −5.1 |
| 157 | Ribosomal protein S18 | 1073 nt DS stop codon | 222 | −2.3 |
| 158 | Ribosomal protein S19 | 70 nt US stop codon | 265 | −6 |
| 159 | Ribosomal protein S2 | 440 nt US start codon | 473 | −10.2 |
| 160 | Ribosomal protein S2 | 74 nt US stop codon | 287 | −7 |
| 161 | Ribosomal protein S2 | 18 nt US stop codon | 332 | −4.8 |
| 162 | Ribosomal protein S4 | 37 nt US stop codon | 360 | −7.7 |
| 163 | Hypothetical protein (rps8) | 212 nt US start codon | 263 | −7.5 |
| 164 | Ycf4 | 58 nt US stop codon | 438 | −17.4 |
| 165 | acetyl-CoA carboxylase, carboxyl transferase | 1281 nt US start codon | 271 | −1.9 |
| | subunit beta | | | |
| 166 | acetyl-CoA carboxylase, carboxyl transferase | 32 nt US stop codon | 941 | −1.4 |
| | subunit beta | | | |

DS = downstream;
US = upstream

TABLE 3
RNA structures sequences (N is any nucleotide and x = 1-100) and
RNAalifold output.

| # | structure |
|---|---|
| 1 | UGUCCU(N)xAU(N)xCCAUUAGACAA(N)xUGGACGCUUUUCGUUGCGAU(N)xU(N)xUUU(N)xUUUCA |
| | UAUUUU(N)xAACUUUCCUACUUA(N)xUGGA(N)xAAAA(N)xGAAA(N)xAAAGAAU(N)x |
| | ..................(((((....)).))).(((........))).................................(((((..........)))...........))......................... |
| | ............... (−11.20 = −11.20 + 0.00) |
| | ..................(((({....}).))).(((........)))..........,,,.,{{{...,{({........(((((...........)))............)),..................}))}. |
| | ......,,,,....... [−12.87] |
| | ..................(((({....)).))).(((........))).................................(((((..........)))...........)).......................... |
| | .............. {−11.20 = −11.20 + −0.00 d = 9.17} |
| 2 | (N)xA(N)xA(N)xAAUUUCG(N)xG(N)xGAAU(N)xU(N)xGUAUAUUCAAUUCUGAAUGGUCUAUUA(N)x |
| | A(N)xU(N)xUUAU(N)xAG(N)xA(N)xCUCU(N)xAAAUAUUCA(N)xUUAGAGUCUA(N)xU(N)xUAGAGU |
| | (N)xCUAUC(N)xAA(N)xAAUAGAAUAGAUAGAAUAGGGCGGGUAG |
| | ..............................................................................((((............))))....(((..((.((((((...................... |
| | ...............................((((...........((.........))))))((((............................))))((....(((((............................ |
| | ........)).)))))..)))))).))..))) (−14.40 = −14.40 + 0.00) |
| | ......................................................................,,,.....{{{,.............\|\|\|\|....,\|}}}\|{.((((({..................... |
| | ........................,,.........((((...........((.........)))))){{{{............................}}}}((....(((((........................ |
| | ............),,)))))..)))))).)} [−17.21] |
| | ........................................................................................................(.((((((.......................... |
| | ...............................((((...........((.........))))))((((...........................))))((....(((.(............................. |
| | ......)..)))))..)))))).)...... {−9.80 = −9.80 + −0.00 d = 17.06} |
| 3 | C(N)xAUCG(N)xUUA(N)xGCUUGGA(N)xAGGCUAG(N)xGGGU(N)xUAUAGUCGACGUCG(N)xAUUCA |
| | AC(N)xG(N)xU(N)xUUUGAGCGUC(N)xUCUAAUUCAAAACCGAAC |
| | ...(((..(((.....((((............)))))))....)))....(((..(((((.......((((................................................................... |
| | ............................)))))))))..))).............. (−15.50 = −15.50 + 0.00) |
| | ...(((..(((.....((({............}))))))....)))....{((..(((((.......((({................................................................... |
| | ............................}))))))))..))}..,,......,,.. [−16.99] |
| | ...(((..(((.....((((............)))))))....)))....(((..(((((.......((((................................................................... |
| | ...........................)))))))))..))) {−15.50 = −15.50 + −0.00 d = 3.99} |
| 4 | (N)xUGC(N)xUAUAGGACAUUGAUUUUGA(N)xAUUCGAU(N)xA(N)xACUUUCA(N)xC(N)xA(N)xAAAA |
| | AUUU(N)xG(N)xGACAUAUU(N)xUUAUAUUUUAC(N)xUUUUUUUAUUAUU(N)xAUGAGAAUGAAU( |
| | N)xCCUACGACUUCUGGUUCU(N)xGGGGUUUCCACCCUUGAAAAA |
| | ......................((((..(((((.........)))))..........................................(((((................))).))...................... |
| | .(((((((.................................................................................................................................. |
| | .......................................................................................................................................... |
| | .......................................................................................................................................... |
| | .....)))))))...............................))))..........(((.......(((((....))))).)))... (−14.20 = −14.20 + 0.00) |
| | ......................{(((..(((((.......)))))............................................{{{{{................}}}.}}...................... |
| | ..(((((((................................................................................................................................. |
| | .......................................................................................................................................... |
| | .......................................................................................................................................... |
| | .......}}}......))))))).,\|,.....................}........,}\|((.......(((((....))))).))}... [−16.55] |
| | ....................((((..(((((........)))))..............................................((((................))).)....................... |
| | (((((((................................................................................................................................... |
| | .......................................................................................................................................... |
| | .......................................................................................................................................... |
| | .....)))).......)))))))...................................((.......(((((....))))).)).... {−12.30 = −12.30 + −0.00 d = 15.35} |
| 5 | AGGCCUUUUAUUUAGUAGGUAACAUCGAUGAAGCUACCGCGAAGGCUAUGAACUUAGAAAUGGAG |
| | AGCAAUUUGAAGAAAUGA(N)x |
| | ..((.((((((((..(((((..(((......((((........))))))).))))).)))))))).)).................. (−10.90 = −10.90 + 0.00) |
| | .(((((((....,.{({({{....,.......,\|))}...))))))).....{{\|.,{{,{{,....)}.}}}.,}}......... [−13.01] |
| | .(((((((.............................))))))).....((...................))......... {−2.04 = −2.04 + −0.00 d = 16.19} |
| 6 | GUUUGGGAUUCA(N)xGAAGUGAAAGAAAUUAUUUUAU(N)xCUACUAAUAGUGGACAAAUUGGCGU |
| | AUUACCCAAUCAC(N)xGCCCCUAUU(N)xGCCACAGC(N)xUGUCGAUAUAGGUAUUUUGAGAAUAC |
| | .........(((.......(((((((((.......)))))).........(((((...))))).......((((((((....))).)).....)))(((((.....(((...)))....)))))..))))))...... |
| | (−17.50 = −17.50 + 0.00) |
| | .........{(({......(((((((({,....,,))))}}.........(((((...})))).......((((((({....})).)).....)))(((((.....{{,...,))....)))))..}}))}}},.,.. |
| | [−19.89] |
| | .........{(({......(..............................(((((...))))).......((((((((....))).)).....)))(((((.....((,...,))....)))))....))))...... |
| | {−13.50 = −13.50 + −0.00 d = 18.65} |
| 7 | AUUUUGAGAAUACG(N)xCC(N)xUUAACGACCAAUGGUUAACGAUGGCUCUG(N)xAUGGGUGGUUU |
| | UGCU(N)xAGAAUAGGUAAUAAU(N)xGA |
| | (((((..(((..((...((..............(((((.((.....)).))).)).......))))..))).....))))).................. (−11.60 = −11.60 + 0.00) |
| | ,,{{,..(((..((...((..............(((((.((.....)).))).))......,))))..))},,,..}},,.,,,............... [−12.92] |
| | .......(((..((...((..............(((((.((.....)).))).)).......))))..)))............................ {−10.40 = −10.40 + −0.00 |
| | d = 8.86} |
| 8 | CUG(N)xAUGGGUGGUUUUGCU(N)xAGAAUAGGUAAUAAU(N)xGAGAU(N)xCACUGUUUUAGUAAA |
| | UAAUGCGGAAAAGAGU(N)xAGUGACAU |
| | ((....((.((...((((((..(((((((.........................)))))))))))))..)).))....))............ (−13.80 = −13.80 + 0.00) |
| | {{....{(.((...((((((..(((((((.........................)))))))))))))..)).)}....}}............ [−14.87] |
| | ((....((.((...((((((..(((((((.........................)))))))))))))..)).))....))............ {−13.80 = −13.80 + 0.00 d = 4.58} |
| 9 | CGAGA(N)xGGAAUGGAAGUGAUUGAUACGGGAGCUCCU(N)xCUA(N)xAGCGUUCCGGUCGGUGGA |
| | GCGACUCUCGGACGAAUUUUC |
| | (((((.................((((((((...............))).)))))((((......)))))))))........... (−18.00 = −18.00 + 0.00) |
| | {{{{{......{(,,{{..({{...\|\|\|{{\|\|\|\|\|...,,.............\|({\|\|\|\|\|,,,\|.})))))),))))))........... [−19.79] |
| | (((((........................................................))))).......... {−2.73 = −2.73 + −0.00 d = 19.00} |
| 10 | GUGGAGCGACUCUCGGACGAAUUUUCAACGUGCUUGGAGAGCCCGUUGAUAAUUUAGGUCCUGUA |
| | GA(N)xUACUCGCACAACAUCCCCUAUUCAUAGAUCUGCGCCCG(N)xCCUUUAUACAGUUAGAU |
| | (((((((((.(((.(((((((((.((((((.((((...)))).)))))).))))).))))...)))............))))..........(((....))).)))))....................... |
| | (−26.60 = −26.60 + 0.00) |
| | (((,,((((.((({(((((((((.((((((.((((...)))).)))))).))))).)))).}.)))............)))).,..,.....,,.....,,,{{({{.................},,)))} |
| | [−29.62] |
| | .....((((.(((((((((((((.((((((.((((...)))).)))))).))))).)))).).)))............))))....................(((....................))). |
| | {−23.00 = −23.00 + −0.00 d = 10.61} |
| 11 | CCAAAGCUCAUGGGGGUGU(N)xAUCUGUAUUUGGGGGAGUAGGUGAACGUACUCGUGAAGGAAAU |
| | GAUCUUUACAAGGAAAUGAAAGAAUCUGGAGUGAUUAAUGAACAAAAUAUUGC |
| | (((((((((....))))........)))))..(((((........)))))((((((........)))))).............((((......))))................. (−16.50 = -16.50 + |
| | 0.00) |
| | {({...{(((((.({{{...,}}}.}\|\|\|}}},.(((((........)))))((((((,......})))))).............{{{{......}}}}................. [−18.97] |
| | ............((......)).......(((((........)))))((((((.......)))))).............((((......))))................. {−10.40 = −10.40 + |
| | −0.00 d = 19.10} |
| 12 | GUUUACGGUCAGAUGAAUGAACCGCCGGGAGCUCGUAUGAGAGUUGGUUUGACUGCCCUAACUAU |
| | GGCGGAAUAUUUCCGAGAUGUUAAUGAACAAAACGUACUUCUAUUUAUCGACAAUAUCUUCCGUU |
| | UCG |
| | .......(((.((((((((..((((((..(((((......)))))(((......))).......)))))).........((((((((.........))))).))).)))))))))))................ |
| | (−25.10 = −25.10 + 0.00) |
| | .....{{((({{{{{{{\|\|,,\|\|\|,({{{(((((......))))){({......\|\|\|....,...}}\|\|\|,...,}}}\|{{\|{{{\|.......,,,},..,},.}}})))))},.....,,,}}}..........}}}.... |
| | . [−28.73] |
| | .......................(((((......)))))...................................................................... {−5.20 = |
| | −5.20 + −0.00 d = 31.37} |
| 13 | UCGUCCAAGC(N)xAGGAUCGGAAGU(N)xAUCUGCUUUAUUGGGUAGAAUGCCUUCCGCUGUGGGU |
| | UAUCAACCCACCCUUAGUACCGAAAUGGGUACU(N)xUUACAAGAAAGAAUUACUUCUACCAC |
| | ..((((..........))))(((((((...........((((((((....)))))).)))).)))))..(((((((...)))))))....((((((......))))))........((((........))))..... |
| | (−35.40 = −35.40 + 0.00) |
| | ..((((..........))))(((((((...........((((((((....))))))},)),})))))..(((((((...)))))))....((((((......))))))........\|\|,.{{,.....))))..... |
| | [−37.91] |
| | ..((((..........))))(((((((...........((((((((....))))))).)).))))))..(((((((...)))))))....((((((......))))))............................. |
| | {−34.50 = −34.50 + −0.00 d = 7.69} |
| 14 | UACUACCGUACUAUCAAGAGGAUUGGCCGCCAAGGGUAUCUAUCCAGCAGUAGAUCCUUUAGAUUC |
| | AACGUCAACUA |
| | ......(((...(((.((((((((.((.((...((((....)))).)).)).)))))))).)))...)))....... (−19.30 = −19.30 + 0.00) |
| | ......(((...(((.((((((((.((.((...((((....)))).)).)).)))))))).)))...)))....... [−19.73] |
| | ......(((...(((.((((((((.((.((...((((....)))).)).)).)))))))).)))...)))....... {−19.30 = −9.30 + −0.00 d = 0.91} |
| 15 | ACUUCAGGACAUUAUAGCUAUCCUUGGGUUGGACGAAUUAUCCGAAGAGGAUCGUUUAA(N)xCCG |
| | UAGCAAGAGCGCGAAAAAUUG |
| | ................(((...((..((((((((((....(((.....)))))))))))..........))..))....)))........... (−19.40 = −19.40 + 0.00) |
| | ......,.........(((.,.{{..((((((((((....(((.....)))))))))))..........))..}},...}))........... [−20.62] |
| | ................(((.......((((((((((....(((.....)))))))))))..........))........)))........... {−18.40 = −18.40 + −0.00 d = 6.81} |
| 16 | AGCGUUUCUUAUCACAACCUUUUUUUGU(N)xAGCAGAAGUAUUUACCGGUUCUCCAGGGAAAUAU |
| | GUUGGUUUAACGGAA |
| | .....................((((((...(((....(((((..((((....)).))..)))))....)))..))))) (−10.30 = −10.30 + 0.00) |
| | ..,,{{....,...{{,,....((((((....}))))}(((((..((((....)).))..)))))}}})}}..})))},. [−12.62] |
| | .....................((((((....))))))(((((..((((....)).))..)))))............. {−8.10 = −8.10 + −0.00 d = 12.27} |
| 17 | GGAGAGCAAUUUGAAGAA(N)xAUGACCUUAAAUCUUUGUGUACUGACCCCUAAUCGA(N)xAUUGU |
| | UUGGGAUUCA(N)xGAAGUG(N)xAAAGAAAUUAUUUUAUCU |
| | ...........((.(((................((((......(((((......(((..(((((.(((....))).)))))..)))..................)))))..))))))).)). (−10.70 = |
| | −10.70 + 0.00) |
| | .,{{..,..{{{,,.{\|\|..................\|\|\|..}}}...{((((......(((..(((((.(({....})).)))))..)))......,........))))}..,,,,,,,.,,. [−13.01] |
| | .........................................(((((.......(((..(((((.((....))).)))))..)).................)))))............ {−9.60 = −9.60 + |
| | −0.00 d = 14.87} |
| 18 | CUAGCCCUCCGACGAGCUAGGACACGCGUAGAGACUAUCAGUGCGAUUUCAUAA(N)xCUAGUUG(N) |
| | xGUGCAUCUG(N)xAA(N)xUAGUC(N)xA(N)xAAAGAAGCUCGGUUUAUACACCCUAUCU(N)xU(N)xUG |
| | UUUGAUUGGAUUC(N)xU(N)xG(N)xUCGAGUAAA(N)xUCCAAUCAA(N)xA(N)x |
| | ..........((((((...((....))...(((((.((((((((((.............................................................................................. |
| | .......................................................)))).........))).))).........)))))..................))))))........................... |
| | .............................................................................(((((((((((..............)).........))))))))).................. |
| | ............ (−27.60 =−27.60 + 0.00) |
| | (((((..{{....}}))))).,,..,,,,...(((((.((((((({,,............................................................................................ |
| | .........................................................,,,}......}))).))).........))))).....................}}..{({......}}}.............. |
| | ..........................................................(((((((((((..................}).......)))))))))............................ [−30.67] |
| | (((((.........))))).............(((((.((((((.................................................................................................. |
| | ................................................................))).)))..........)))))........................(((......))).................... |
| | .......................................................................(((((((((((..................}).......)))))))))........................ |
| | ......{−26.30 =−26.30 + −0.00 d = 13.13} |
| 19 | (N)xU(N)xUGUUUGAUUGGAUUC(N)xU(N)xG(N)xUCGAGUAAA(N)xUCCAAUCAA(N)xA(N)xUACAA |
| | UCAAGCG(N)xGAAUCCUAUUUUGAUUCAACAAAAAAAAU(N)xGAAAAUUCCAAAAUGAA(N)xAUA(N |
| | )xGAAA(N)xAG(N)xAU(N)xAUAAA(N)xAUCAAU(N)xAUC(N)xAAUAUAAUAUCAAU(N)xAUAA(N)xAG |
| | AAG(N)xAUGGGUA(N)xUACAAACUUAUUAGAU |
| | ..........................(((((((((((..........)).....))))))))..........................................(((((.......)))))..................... |
| | .............................................................................................................................................. |
| | ........((((((......................))))))..... (−16.60 = −16.60 + 0.00) |
| | ..........................(((((((((((..........}).....))))))))..........................................{{{{{...,,,,}}}}}..................... |
| | ...........,.........},.............................................................,.......,,,............................................... |
| | .........{(((((......................)))))}..... [−18.95] |
| | ..........................(((((((((((..........)).....))))))))................................................................................ |
| | ............................................................................................................................................... |
| | .....((((((........................))))))..... {−13.60 = −13.60 + −0.00 d = 16.09} |
| 20 | (N)xAUCUCAUAU(N)xUAUUUAUAA(N)xG(N)xCAAUACCAAU(N)xCU(N)xAAGCAAUAC(N)xCACUCA |
| | AACAGAU(N)xCAAAGAGCUAUGAU(N)xGUU(N)xAGAUCAUG(N)xG(N)xAAUAUUC(N)xAUCU(N)xU |
| | GACAAG |
| | ............................................................................................................................(((...((((..((..... |
| | ((((((.....................)))))).......)).....................)))).))).... (−4.70 = −4.70 + 0.00) |
| | ..........................................................................................................................,{(....,,,,..,,.,.,, |
| | .\|{{(({.....................))}})).......,,.....................,,,,.,,...., [−6.22] |
| | ..........................................................................................................................,{(................( |
| | (((({......................)))))).......................................... {−4.00 = −4.00 + −0.00 d = 11.00} |
| 21 | (N)xAUCU(N)xUGACAAGAAAU(N)xUAUCUACAUGAU(N)xAAAAUAUGUAUCACAA(N)xGCACU(N)xA |
| | AGG(N)xGCUAUAGCUCAGU(N)xUAGGUAGAG(N)xC(N)xAACUCGU |
| | .......((.((((.....................((((((...................................))))))...........................((.((....)))).))...))))..(((...... |
| | ..)))..(−9.60 = −9.60 + 0.00) |
| | .......((.((((..,,...............,,((((((...................................}))))}...........................((.((....)))).))...))))..{{{....... |
| | .}))..[−10.76] |
| | .......((.((((.....................((((((...................................))))))...........................((.((....)))).))...))))..(((...... |
| | ..)))..{−9.60 = −9.60 + −0.00 d = 8.48} |
| 22 | AUCACAA(N)xGCACU(N)xAAGG(N)xGCUAUAGCUCAGU(N)xUAGGUAGAG(N)xC(N)xAACUCGUUUA |
| | C(N)xACGUGCGCCAAUGUUUU(N)xUC(N)xAG(N)xGGGAGUC |
| | .........((((...........((.((....)))).........(((((.............))))).........))))................................... (−10.20 = −10.20 + 0.00) |
| | .........((((...........((.((....)))).........(((((.............))))).........))))..............................,.... [−11.03] |
| | .........((((...........((.((....)))).........(((((.............))))).........))))................................... {−10.20 = −10.20 + −0.00 |
| | d = 5.16} |
| 23 | UUUU(N)xUC(N)xAG(N)xGGGAGUCCAU(N)xCAUGCA(N)xAUCAAAAGA(N)xA(N)xUUG(N)xAU(N)xC |
| | UUAUUGAG(N)xAA(N)xAUCAAUG(N)xUCUUAC(N)xUCC(N)xAUAACUU(N)xUGAGGGAAC(N)xA(N)x |
| | A(N)xGAGA |
| | .................(((((.....(((..........(((((((......................................))).))))..............))).....)).)))......................... |
| | ............(−9.40 = −9.40 + 0.00) |
| | .,,,.....,,......(((((.....,,,.........,((((,,,....................,}}.....}}........,,\|{\|\|\|\|..............})}))).....)).))).............,,}},,... |
| | .................[−11.03] |
| | .................(((((..................(((.........................)).....)...........(((((............)))))......)).)))......................... |
| | ...........{−6.30 = −6.30 + −0.00 d = 13.40} |
| 24 | (N)xA(N)xA(N)xAUAUCUA(N)xU(N)xAAAAA(N)xAA(N)xAAGAAAAGGGGG(N)xC(N)xG(N)xAAGU(N)x |
| | GA(N)xUACAAAAA(N)xG(N)xAAC(N)xUCUGUUC(N)xGAUU(N)xUU(N)xUU(N)x |
| | .............................................................................................................................((((..((...(((.(((... |
| | ..............................................))).)))........))..))....)).. (−1.40 = −1.40 + 0.00) |
| | .............................................................................................................................,{((..((...(({.{((,.. |
| | ..............................................})).)))........))..))....)}.. [−2.74] |
| | .............................................................................................................................(((..((...(((........ |
| | .................................................)))........))..))....)... {1.33 = 1.33 + −0.00 d = 8.15} |
| 25 | UCUGUUC(N)xGAUU(N)xUU(N)xUU(N)xAGUCUAU(N)xCUAUAAGAGGAG(N)xAUCCU(N)xAUGAAA |
| | AAU(N)xGUAACCGAUUCCUUCGUUUCUUUG(N)xGGCCAC |
| | ..............................................(((((..............((((((.........((................................................................ |
| | ...........................................................................))))))))........)).........)))... (−7.50 = −7.50 + 0.00) |
| | ..............................................(((((..............(((((,........,((................................................................ |
| | ...........................................................................))))))))}.......)).........)))... [−8.79] |
| | ..............................................(((((..............((((...........((................................................................ |
| | .............................................................................)).)))).......)).........)))... {−4.10 = −4.10 + −0.00 |
| | d = 5.57} |
| 26 | AGAU(N)xGAAUUUG(N)xUA(N)xGCGA(N)xU(N)xGG(N)xAAUAAUUAAGUC(N)xAUAAU(N)xUCA(N)x |
| | UU(N)xGAUUGUA(N)xUC(N)xAUU(N)xAACUAUUUCUAUUUUU(N)xUU(N)xAUACGAG(N)x |
| | ............................,((...........((({((((.......................................))....}}}}}}.............)).............................. |
| | ...........................(−3.30 = −3.30 + 0.00) |
| | .........{{{....{,.....,.....((..........,((({((((.......................................,}....}}}}}}.............,,.................,,..,.,,,,,, |
| | ,,,........}}}}}}........ [−4.67] |
| | ................................................................................................................................................ |
| | ......................{0.00 = 0.00 + −0.00 d = 13.95} |
| 27 | U(N)xUCA(N)xUU(N)xGAUUGUA(N)xUC(N)xAUU(N)xAACUAUUUCUAUUUUU(N)xUU(N)xAUACGA |
| | G(N)xGAACUUAU(N)xC(N)xAUGAAUCCACUGAUUUCUGCCGCUUCCGUUAUUGCUGCUGGAUUGGC |
| | UGUAGGGCUUGCU |
| | .........................................................................((((................................................................ |
| | ............................................................................................................................................. |
| | ..................(((((....)))))((((.((((((((.......)).)))..))).))))..)))).. (−11.60 = −11.60 + 0.00) |
| | ..............................................................,,,........,\|\|\|................................................................ |
| | ............................................................................................................................................. |
| | ....................,{{{{....}}}}\|((((.((((((({.......)},)))..))).))))}.,,,,.. [−14.08] |
| | ............................................................................................................................................. |
| | ............................................................................................................................................. |
| | ..................((((....)))).((((.((((((((.......)).)))..))).))))......... {−9.80 = −9.80 + −0.00 d = 14.98} |
| 28 | AAGAGA(N)xUGA(N)xU(N)xU(N)xGA(N)xAUCA(N)xAAAUA(N)xAU(N)xUUCGU(N)xUUAAA(N)xGUC |
| | UGAUUUUU(N)xCUCAGAGGUCAAU(N)xAUGAAUGUUCUAUCAUGUUCCAUCAA |
| | ....((.((..................................................(((((....................)))))...................................................... |
| | ........................................................................................................................((((((......)))))))))). |
| | . (−7.50 =−7.50 + 0.00) |
| | ....((.((...........{{.,,....,,,.............,,...,,.......{{(((,,,,................}}))),,,,,............................................... |
| | .........................................................................................................................,(({{{,....,}))}))))))).. |
| | [−9.29]....((.((..................................................(((((....................)))))................................................. |
| | ............................................................................................................................................)))). |
| | . {−6.97 =−6.97 + −0.00 d = 13.81} |
| 29 | CAGAGGUCAAU(N)xAUGAAUGUUCUAUCAUGUUCCAUCAAC(N)xACA(N)xCUAAAG(N)xGGGUUAU |
| | ACGAUAUAUCCGGCGUAGAAGUAGGCCAACAUUUC |
| | ................................................................................................................................................ |
| | ...... ...............(((((((................................................................................(((..........))))(((.((....)).)))) |
| | )))))).(−10.60 = −10.60 + 0.00) |
| | ............................................................................................................................................... |
| | ....... ...............(((((({......{{,,.....................................................................{((,,{{,...})})}))(((.((....)).))) |
| | })))))}.[−12.89] |
| | ................................................................................................................................................ |
| | ...... ...............(((((((...............................................................................(((..((.....)).)))(((.((....)).)))) |
| | )))))).{−6.80 = −6.80 + −0.00 d = 11.73} |
| 30 | GCGCGG(N)xCUAC(N)xAAAUAAU(N)xCUAU(N)xUUAGGGAAUAUAAAUAGACAAC(N)xUAC(N)xAC( |
| | N)xUCU(N)xAGAC(N)xGAUC(N)xUAGA(N)xAUAAUAGCAAAA(N)xAAGAUUAC |
| | ......................((((.(((.......)))))))...........................................................................................(((....(( |
| | ..........................)).......)))................................... (−4.60 = −4.60 + 0.00) |
| | ......................((((.(((.......)))))))...........................................................................................(((....({ |
| | ..........................}).......)))....................},............. [−5.73] |
| | ......................((((.(((.......)))))))...........................................................................................(((....(( |
| | ..........................)).......)))................................... {−4.60 = −4.60 + −0.00 d = 3.35} |
| 31 | GAUUGAAUUA(N)xU(N)xGA(N)xAU(N)xAGA(N)xCU(N)xAUUA(N)xUGAGUUCUAGAAAGC(N)xCGCC |
| | AUGGUGAAAUCGGUAGACACGCUGCUCUUAGGAAG |
| | ......................................................((((((..(((....((....(((...((....))))))).))).)))))).. (−7.90 = −7.90 |
| | + 0.00) |
| | ...................,,....\|\|...........................}}\|\|{{,{,.{((....({..,\|\|{{...\|\|,.\|,\|,,,,}),))).}},}},.. [−9.89] |
| | ..............................................................((....((...............)).)).......... {−1.10 = −1.10 + |
| | −0.00 d = 15.70} |
| 32 | AA(N)xUAAAUUCAAUU(N)xCUUG(N)xAUU(N)xUCU(N)xAU(N)xUUCG(N)xUAA(N)xU(N)xUAGUAA( |
| | N)xUUAACGGAU(N)xUCC(N)xUC(N)xUU(N)xU(N)xU(N)xAUGAUAUUUUCA(N)xAUUCUAGAGCAUA |
| | UAUUAACUCAUAUUUCCUUU |
| | ...........................................................................................................(((........)))...................... |
| | .............................................................................................................................................. |
| | .............................................................................................................................................. |
| | ....................................................................................((((((.(((.............)))...))))))............... |
| | (−2.00 = −2.00 + 0.00) |
| | ...........................................................................................................(({........}}}..................... |
| | .............................................................................................................................................. |
| | .............................................................................................................................................. |
| | ......................................................................,{(.....\|\|.............\|\|{...\|\|,.....,............ [−4.20] |
| | ...........................................................................................................(((........)))..................... |
| | .............................................................................................................................................. |
| | ................................................................................................................................. |
| | {−1.10 = −1.10 + −0.00 d = 10.96} |
| 33 | CUACAUAGUUAUGGUUCAUUUACGUUAACGUCUAAUUGA(N)xAUUCAAGA(N)xA(N)xAGGUUCU(N |
| | )xU(N)xUCAAAUA(N)xCA(N)xA(N)xACAGA(N)xGUA(N)xGAG(N)xUGUAUA(N)xA |
| | .......((((..((......))..)))).(((..((((......................................................................................................... |
| | ......))))........)))...............................................................................................................(((......... |
| | ...........)))..... (−3.40 = −3.40 + 0.00) |
| | .......((((..((......))..)))).,{,..{(({......................................................................................................... |
| | ......,)}}........,,................................................................................................................,,,......... |
| | ..........}}}..... [−5.53] |
| | .......((((..((......))..))))......(((......................................................................................................... |
| | .......)))..................................................................................................................................... |
| | ................... {−2.00 = −2.00 + −0.00 d = 9.32} |
| 34 | GA(N)xAAAAUG(N)xAUCCACGGAUAUGAA(N)xUCCAAUU(N)xAUAA(N)xUC(N)xUUUGUGACUUUG |
| | UAAUUGGAGAUAAG(N)xCA(N)xAA(N)xGA(N)xA(N)xAAUCAUACUUACU(N)xCUUUAUA(N)xUU |
| | .........................................................................(((....)))..........(((((((.....((((.....................((.......... |
| | .....................))..))))))))))).................................................................................................... |
| | (−11.00 = −11.00 + 0.00) |
| | .........................................................................{({....}}}.,........(((((((.....((((.....................((.......... |
| | .....................))..)))))))))))..,,..................................,............................................................. [−12.57] |
| | ..........................................................................((....))...........(((((((.....((((.....................((............ |
| | ...................))..))))))))))).................................................................................................... {−9.40 = |
| | −9.40 + −0.00 d = 6.09} |
| 35 | UUGCUUU(N)xCAUUUA(N)xAUGCCUAUUGGUGU(N)xUC(N)xCAAA(N)xACU(N)xAGCGUUUCUAAU |
| | (N)xUC(N)xCUGGAGACGAAGAAGCGACUUG(N)xGGUUGA(N)xCAUAUAGUGCGAC(N)xUUGUC |
| | (((((((..................(((((....)))))..................((((((((........))))))))..)))))))..................((((...............(((...))))))) |
| | ............................................................................................................................................ |
| | ............................................................................................................................................ |
| | ............................................................................................................................................ |
| | ............................................................................................................................................ |
| | ................................................................ (−18.80 = −18.80 + 0.00) |
| | {((((((..................{((((....)))}}..................((((((((........))))))))..))))))}..................((((...............(((...))))))) |
| | ............................................................................................................................................ |
| | ............................................................................................................................................ |
| | ............................................................................................................................................ |
| | ............................................................................................................................................ |
| | ................................................................ [−19.78] |
| | (((((((..................(((((....)))))..................((((((((........))))))))..)))))))..................((((...............(((...))))))) |
| | ............................................................................................................................................ |
| | ............................................................................................................................................ |
| | ............................................................................................................................................ |
| | ............................................................................................................................................ |
| | ................................................................ {−18.80 = −18.80 + −0.00 d = 7.28} |
| 36 | UAGCC(N)xCAGCCC(N)xCAGA(N)xUUUCGC(N)xGCAAAUUCGC(N)xGAU(N)xUUUCU(N)xCUUCUUA |
| | ACUG(N)xA(N)xG(N)xUUU(N)xA(N)xAUAUUCUAU(N)xUAAAUAUA(N)xGAAUA(N)xUUAAAUACAA |
| | GCUACAAGCAAUUCCUAAUCAUGCAGU(N)xU |
| | ................(((..((((............))..)).............)))........((((............((((((((......))).....)))))...........(((...)))........... |
| | .......))))......... (−13.30 = −13.30 + 0.00) |
| | .,{{......,.....{((..((((............))..))............,}},.........,{{.....,,,....((((((((......))).....)))))...,,,.....\|\|\|...,\|\|,......... |
| | ........,})............ [−15.61] |
| | ................(....((((............))..)).............)..........................((((((((......))).....))))).............................. |
| | ................. {−7.90 = −7.90 + −0.00 d = 10.89} |
| 37 | (N)xCAUGCCAACUAUUAAACAACUUAUUAGAAAUACAAGACAGCCAAUCAGAAAUGUCACAAAAUCC |
| | CCCGCCCUUCGGGGAUGCCCUCAGCGUCG(N)xAGGA |
| | .......((..(((.(((.....))).)))........((((............)))).........((((.....))))(((((.....)))))...)). (−15.10 = −15.10 + 0.00) |
| | .......({..(((.(({.....})).)))........((((............)))).......,,{(({{.....})))\|\|\|((.....)))),...)). [−16.31] |
| | .......((..(((.(((.....))).)))........((((............)))).........((((.....)))).((((.....)))))...)). {−11.30 = −11.30 + 0.00 |
| | d = 10.12} |
| 38 | GA(N)xG(N)xGGACAGGUUGU(N)xCUA(N)xAU(N)xACC(N)xGU(N)xCCC(N)xUCCUCUAUUUG(N)xUU |
| | AA(N)xUACUAA(N)xUACUUCUACUUCAGGG(N)xAGGAUUUAUUUAAA(N)xAUGGGCGAAAACGAAA |
| | AAAAAUCUAUUCAUGAGGGUUUAGUUACU(N)xG |
| | ((...((((.(((..............)))..)).))......))..............................(((....((((((((.....((((((.(((.............................. |
| | .................................................................................................................))).)))))).))).))))).. |
| | ..))).............(−18.60 = −18.60 + 0.00) |
| | ((...((((.(((..............)))..)).))......)).......................,,{{\|..{((....{{((((((.....((((((.{{{.............................. |
| | .................................................................................................................,}}.)))))).))).))))).. |
| | ,,))}.............[−20.51] |
| | ((...((((.(((..............)))..)).))......)).....................................((((((((.....((((((.................................. |
| | .....................................................................................................................)))))).))).))))).. |
| | ..................{−17.00 = −17.00 + −0.00 d = 13.38} |
| 39 | UAAGUCGUUAU(N)xGAUGCAACCAAAGGGCGUAUAAUUUAUCGACUUCGCGAUAAGGAUUCGAAA |
| | GAUUAG(N)xGUG(N)xUUU(N)xUUUUUUUC(N)xAACUU(N)xUC(N)xACAUUCCUUU(N)xCGU(N)xG( |
| | N)xGGAAUA |
| | .((((((.((.....((((..((...))..))))....)).)))))).(((..(((((...(((((....................................................................... |
| | .......................................................................................................................................... |
| | .......................................................................................................................................... |
| | .................................................................................))).))......)))))..))) (−18.70 = −18.70 + 0.00) |
| | .{(((((.{(.....((((..((...))..))))....}}.)))))}.(((..(((((...(((((....................................................................... |
| | ......................................................................................................................................... |
| | .......................................................................................................................................... |
| | ..................................................................................))).))......)))))..))) [−20.20] |
| | .((((((........((((..((...))..)))).......)))))).(((..(((((...(((((......................................................................... |
| | .......................................................................................................................................... |
| | .......................................................................................................................................... |
| | ...............................................................................))).))......)))))..))) {−18.20 = −18.20 + −0.00 d = 5.96} |
| 40 | (N)xAUUUU(N)xUUUAA(N)xCGAAUCCUA(N)xAUUA(N)xUUGCCAUUUUUUAA(N)xUAUA(N)xU(N)x |
| | A(N)xU(N)xUUUAAAUA(N)xA(N)xUAAAAAUAUA(N)xUUAUGCA(N)xAU(N)xG(N)xGAGAUAUGUG(N |
| | )xU(N)xCUA(N)xAAAGAA(N)xACAUU(N)xAUAU(N)xUG(N)x |
| | .....((((................................((((................................(((((................................)))))................... |
| | .(((((..((..............................)))))))..................)).)))))).................... (−2.80 = −2.80 + 0.00) |
| | .....,{{{................................{{{{.........................,......{{(({.,,.............................}}},}................... |
| | .{{{{{..{{..............................}))}))}..................}}.}}))},.................... [−4.48] |
| | ......(((................................((((.....................................................................))))).................. |
| | (((((..((................................)))))))..................)).)))))).................... {0.09 = 0.09 + −0.00 d = 15.45} |
| 41 | ACAUU(N)xAUAU(N)xUG(N)xA(N)xUA(N)xAAAAAAU(N)xUUCCAAAAUCAAA(N)xAGAGC(N)xGAUU |
| | GGG(N)xU(N)xUGAAAAAAUAAA(N)xGGAUUUCUAACCA(N)xC |
| | ........................................................................((((................))))((................((((.................. |
| | .....))))...))... (−3.40 = −3.40 + 0.00) |
| | ...................................................................,(({..{((................))))}),...............,,,........ |
| | ............,,..,,,...))... [−5.11] |
| | ....................................................................(((..(((................))))))............................... |
| | ....................... {−2.60 = −2.60 + −0.00 d = 7.36} |
| 42 | (N)xUUAUAACGAA(N)xCA(N)xUA(N)xAA(N)xCCA(N)xACG(N)xAAAU(N)xGAAAUAGAAAAACAGAGG |
| | A(N)xUAGA(N)xGAAUC(N)xUGUUGA(N)xUAAGUCUA(N)xCCU(N)xAUAUCCGA(N)xGG(N)xUAUCUA |
| | UUCUUU(N)xUUCUUAUUUCUUAUAAGAAAAGAAU(N)xACCUUGUUUUGACUGUAUCGCACUA(N)x |
| | UGCAU(N)xCAUU(N)xUGAGAACCCCCCAAAUCCUCUACUUUUGGU(N)xUGAAAUCAAAUUUCAA(N)x |
| | AUGGAGGAA(N)xUUCCAA(N)xAGAUAU(N)xUUAAAGAUA(N)xGAUGGGUCUCAACAACAC(N)xUAC |
| | UUC |
| | ....................................................................................................((.((((.(((((((((((...((((.....((........ |
| | ..((.....((((..................))...)))).))..........)))))))))...........................................................(((((((.....))))))). |
| | )))).))(((.......)))............(((((.(((.(((((.......)))))..................................((((((...))))))................................. |
| | ............................................................................................................................................. |
| | ............................................................................................................................................. |
| | ............................................................................................................................................. |
| | ............................................................................................................................................. |
| | ............................................................................................................................................. |
| | ...............................................((((........))))..............................))))))))..................................... |
| | (−37.60 = −37.60 + 0.00) |
| | ....................................................................................................((.((((.(((((((((((...((((.....{(..... |
| | ((.....(({{..................}}...)))).}}.....,,...)))))))))...........................................................(((((((...,.. |
| | ....))))))).,,.......,})))))..)))).))(({.......}))............(((({,(((.(((((.......))))).................... |
| | ..............((((((...))))))................................................................................................................... |
| | ............................................................................................................................................... |
| | ............................................................................................... |
| | ........................................................................................................................................... |
| | ............................................................................................................................................ |
| | ........................................................................................................................................... |
| | ..............(((({.......,)))..............................))))))))..................................... [−42.05] |
| | ....................................................................................................((.((((.(((((((((((...((((............. |
| | ....((.....((((..................))...)))).............)))))))))...........................................................(((((((.....))) |
| | ))))..........))))))..))))).))(((.......)))............(((((.(((.(((((.......)))))..................................((((((...))))))....... |
| | .......................................................................................................................................... |
| | ...................................................................................................................... |
| | .................................................................................................................................. |
| | .............................................................................................................. |
| | ......................................................................................................................... |
| | ................................................................................................................................................. |
| | ...............((((.........))))..............................))))))))..................................... {−37.20 = −37.20 + −0.00 d = 21.22} |
| 43 | (N)xGAA(N)xACUAU(N)xG(N)xUAAUUGGAAAUUCU(N)xUCCA(N)xA(N)xAAUUAUG(N)xAA(N)xUAU |
| | (N)xAA(N)xAU(N)xA(N)xAUGAUA(N)xG(N)xAGAUAA(N)xU(N)xGAUAACA(N)xA(N)xA(N)xAAUUUG |
| | GAUUUUUUUUUUAUUCUGA(N)xAACGU(N)xUGAUGUAGCA(N)x |
| | ........................................................................................................................................... |
| | ........................................................................................................................................... |
| | ............((((................)))))).................((((.................))))............(((((....................................))))). |
| | ......................................(((((...........)))))..........))))... (−4.30 = −4.30 + 0.00) |
| | ........................................................................................................................................... |
| | ........................................................................................................................................... |
| | .............,,,.....................((((.................))))............,{{{{....................................,,,}}.......,........... |
| | ....................,\|\|,{......,.,.}},},..,.,,....,})},... [−7.14] |
| | ........................................................................................................................................... |
| | ........................................................................................................................................... |
| | .....................................((((.................))))............................................................................. |
| | .......................................................... {−3.40 = −3.40 + −0.00 d = 13.67} |
| 44 | UGGAGAUUCCGUA(N)xAAUAUUCCAUUCCAAAAAUGG(N)xAAAGUUCGA(N)xAACAAUUG(N)xGGA |
| | UUUU(N)xUUUGGAA(N)xAUGG(N)xGAUGCAGUUACU(N)xAAUUC(N)xAUGAUCUGGCAUGUACAG |
| | AAUGAAAACUUCAUUC |
| | .(((...)))........((((((((((((((((..........................................................................................))........)))) |
| | ))....))))...))))((((((................................................................................................................... |
| | .........))).)))........(((((((...))))))) (−20.00 = −20.00 + 0.00) |
| | ,(((...}))\|.......{(((((((((((((((............................................................................................))........)) |
| | ))),...,)))...)))\|{{{{{{.................................................................................................................. |
| | ............,,,.\|\|\|.,.....,.(((((({...})))))) [−23.04] |
| | .(((...)))........((((((.(((((((((..........................................................................................))........))))) |
| | )....)))...)))............................................................................................................................. |
| | ...................((((((.....)))))) {−16.20 = −16.20 + −0.00 d = 12.54} |
| 45 | UGUGUGAUAGCAUCUACU(N)xAUACCAGGAAUAUCAAUGAACCCGAUUAUUGCAAUUGCUCAGGAU |
| | ACCCUUUUUUAG(N)x |
| | .((((....))))................((.(((..((((((......))))))..))).)).(((....)))............................................................. |
| | ......................................................................................................................................... |
| | .................................................................................................................................. |
| | ...................................................... (−7.70 = −7.70 + 0.00) |
| | .(,((.,{{{......}}......}},},((,{{(,,({{(((...,,,\|\|}}}}..))),}),\|{\|,,,.))}....................................................... |
| | ............................................................................................................................................... |
| | ............................................................................................................................................... |
| | ............................................ [−9.72] |
| | .......(((......))......).....(.(((..((((((......))))))..))).)........................................................................ |
| | .............................................................................................................................................. |
| | ............................................................................................................................................... |
| | .................................. {−3.00 = −3.00 + −0.00 d = 17.25} |
| 46 | CCGAUUAUUGCAAUUGCUCAGGAUACCCUUUUUUAG(N)x |
| | .........((....))..(((....)))........................................................................................................... |
| | ............................................................................................................................................. |
| | ............................................................................................................................................. |
| | ........................................................................... (−1.90 = −1.90 + 0.00) |
| | .........{{....)}..(((....)))............................................................................................................ |
| | ....................................................................................................................................... |
| | ........................................................................................................................................... |
| | .................................................................................. [−2.61] |
| | .........((....))..(((....)))...................................................................................................... |
| | ....................................................................................................................................... |
| | ............................................................................................................................................ |
| | ............................................{−1.90 = −1.90 + −0.00 d = 3.46} |
| 47 | (N)xAAAA(N)xAAA(N)xAUA(N)xACAAG(N)xA(N)xUGG(N)xCA(N)xAUAAGC(N)xAA(N)xAGAUAAUAA |
| | AA(N)xA(N)xUGGAUUU(N)xCAAUUU(N)xCGAUC(N)xAAU(N)xUU(N)xCUA(N)xAU(N)xAAGAAUUC( |
| | N)xCAU(N)xAU(N)xUAGA(N)xAUU(N)xCCCUU(N)xU(N)xCUUUAUAGAGAAUCC(N)xU(N)xA(N)xGGU |
| | G(N)xA(N)xGAU(N)xGAAAACAAGAGA(N)xAUUU(N)xGUAUAAG(N)xAACA |
| | .................................................................((((............))))......((.((.......)))).............................. |
| | ...................................((((((.(((.((..............((.............))....)).....)))..........)))))).................. |
| | ..................... (−6.00 = −6.00 + 0.00) |
| | ...........................................................{(({{...........,},,......,{.((,......\|\|\|\|,{{............................ |
| | ......................}}......}},......,{{{{{{.{{{.((..............{{.............}}....}}.....)))..........))}}}}.................. |
| | ..................... [−8.90] |
| | ................................................................................................................................... |
| | .........................................((((((.(((.((..............((.............))....)).....)))..........)))).................... |
| | ...................{−3.10 = −3.10 + −0.00 d = 19.27} |
| 48 | ACA(N)xAGAGUU(N)xAGUUAU(N)xACUAAC(N)xUAUA(N)xACUAAAUC(N)xAAA(N)xU(N)xCA(N)xA |
| | AAU(N)xAC(N)xGU(N)xAUAAUAGAAAUCG(N)xGA(N)xU(N)xUC(N)xCAUUUC(N)xAGUC(N)xG(N)xA |
| | GGAAUCU(N)xUGA(N)xAAUGAGAU(N)xAUGCCCCACAG(N)xUAAGA(N)xAAAGGAAACUA(N)xGACG |
| | (N)xG(N)xUU(N)xUG(N)xAUC(N)xAAAAUCAAUUCU(N)xUGUU(N)xUUGA(N)xCUA(N)xA(N)xACAGU |
| | UAUGGAUG(N)xA(N)xCUUGACAA(N)xUUUCAAGUCGAAUCGACU(N)xAAUCCGGU(N)xAUGGUAUA |
| | U(N)xUUU(N)xCCACGU(N)xUCAUAG(N)xG(N)xA(N)xG(N)xUGUGUCU(N)xAUGUUUCUGCUUUACG |
| | AAUAUGAUAUUUUUUGGGCGUUUCUAAUAAUUUCAAG |
| | ...........................((.((.((((((.........)))..........))))).)).....(((((..........................................(((((..... |
| | .)))))............................................((......(((........(((.(((...((((((.............................((....)).......... |
| | .................((.(((((...........))).))))......................................(((((((((........(((((.......................... |
| | )))))((((..............))))....(((........))))))...)))))).......................................................................... |
| | ..................................................................................(((((............................................. |
| | .................................................................................................................................. |
| | ................................................................................ |
| | .............................................................................................................................. |
| | ))))).....)))))).)))....)))))))) (−41.40 = −41.40 + 0.00) |
| | ...........................((.((.((((((.........)))..........))))}.))....................................................{{{{{..... |
| | \|\|\|\|\|.............................................((......(((........(((.(((...((((((...,..,.,,,...............,.,(,,,\|,.......,..... |
| | ,,.........,.,,...((.(((({,,...........))).))))....,..................................{((((((((........(((({.........,,,)}}},{{( |
| | {..........)))}....{((.....,,,.................))})))...))))),.......................................................................... |
| | ....................................................................................,,................................................... |
| | .................................................................................................................................... |
| | ........................................................................................................................................... |
| | ..........................\|\|}}}}.......\|\|\|,,...,,}},...)))))).)))....}))))))) [−47.25] |
| | ...........................((.((.((((((.........)))..........))))).))....................................................(((((.....)))))..... |
| | .........................................((.....(((.........(((.(((...(((((............................................................. |
| | ((.(((((...............).))))........................................((((((((........((((.......))))......((((...................))))....((( |
| | ................))))))...)))))............................................................................................................... |
| | ......................................................................................................................................... |
| | ......................................................................................................................................... |
| | ..................................................................................................................................... |
| | .......................................)))))).))....)))))))) {−37.40 = −37.40 + −0.00d = 35.11} |
| 49 | CUUUCAUUUUCGUGCUUAUCUUAAUUGUUGGUUCAGUUUAU(N)xGCAUGGCGAAAAGGGGCAUU |
| | GGAAUGGUCUUAG(N)xCUC |
| | ...(((((((.(((((..((((..(((((.((............))..))))).))))))))).)))))))............................................................. |
| | .................................... (−12.40 = −12.40 + 0.00) |
| | ...({(((((.(((((..,(((..(((((,((............)).,))))).))))))))).)))))),............................................................. |
| | .................................... [−14.38] |
| | ...(((((((.(((((..((((..(((((.((............))..))))).))))))))).)))))))............................................................. |
| | .................................... {−11.60 = −11.60 + −0.00 d = 11.31} |
| 50 | GUUAUUCUAUCUAAUUAUUUUAUAGAGAGUUUACAGAAAUAA(N)xA(N)xA(N)xAAAUCCUAUGAU |
| | U(N)xU(N)xUU(N)xUAAACCCUUUGUUG(N)xGAUACU(N)xGAAAAAAAAAAGAAGUCUUUUU(N)x |
| | ....(((((((((.......((((((.(((((....................................................................................................... |
| | ................................................................................................................................ |
| | .............................))))).))))))))....))))....)))...((((((....))))))....................... (−8.00 = −8.00 + 0.00) |
| | ....{{(((((({,......,{((((.(((((......................................................................................................... |
| | ....................................................................................................................................... |
| | ....................))))).))))))))....))))....}},...{(((((....)))))}....................... [−9.94] |
| | ....(((((((((.......((((((.(((((..................................................................................................... |
| | ............................................................................................................................................. |
| | ..................))))).))))))))....))))....))....((((((....))))))....................... {−6.80 = −6.80 + −0.00 d = 7.12} |
| 51 | (N)xCU(N)xUCUUA(N)xUAUUC(N)xGAUUC(N)xAAUCGAAUCGG(N)xU(N)xUAGA(N)xAUU(N)xGUU |
| | GU(N)xUCAUAU(N)xUGAAAUGAAUC(N)xGAGAUUG(N)xAUAAG(N)xGAGUUGUUUA |
| | ........(((.......((...(((((.....))))))).....)))......(((........((.........((((.....................))))))....)))......... |
| | .................................... (−10.70 = −10.70 + 0.00) |
| | ........,({,...,,.((...(((((.....)))))))....,},,......,{,........((.........((((.....................)))))).... |
| | }},..................,,,,....................... [−12.70] |
| | ..................((...(((((.....))))))).........................((.........((((.................... |
| | .)))))).................................................... {−9.70 = −9.70 + −0.00 d = 9.38} |
| 52 | A(N)xAUU(N)xGUUGU(N)xUCAUAU(N)xUGAAAUGAAUC(N)xGAGAUUG(N)xAUAAG(N)xGAGUUGU |
| | UUA(N)xAUGAUGCUCGAACAUGUACUUGUUUUGAG |
| | ........(((......((......((((...............................))))))....))).......................((((.................................... |
| | ............................................................................................................................................... |
| | ................................................................................................................................................. |
| | ............................................................................................................................. |
| | ))))(((((......))))).... (−8.50 = −8.50 + 0.00) |
| | .........,,......((......((((...............................))))))....\|}}...........,,..........{{{,,,{{...................................... |
| | .............................................................................................................. |
| | .......................................................................................................................................... |
| | ........................................................................................................................... |
| | .................................,,.,\|\|\|{((((......))))),},. [−10.51] |
| | .................((......((((...............................))))))........................................................................ |
| | ........................................................................................................................................... |
| | .............................................................................................................................................. |
| | ................................................................................................................................... |
| | ((((......))))..... {−4.20 = −4.20 + −0.00 d = 11.89} |
| 53 | AAUCAACUCGUAUCAAUCAAUCAAAUUUGUUGAAUAAGUAG(N)xUA(N)xUU(N)xAAUC(N)xA(N)xU( |
| | N)xAUA(N)xA(N)xAUU(N)xCUAA(N)xUA(N)xUCU(N)xAA(N)xUAU(N)xU(N)xU(N)xA(N)xU(N)xA(N)x |
| | A(N)xAUCAAUU(N)xCAAA(N)xU(N)xCAGCA(N)xUGU(N)xCU(N)xGA(N)xCACGUA(N)xAGGGAUGAU |
| | A(N)xAUCUUU(N)xG(N)xCACAAAGAUAA(N)x |
| | .....(((.((.((((.(((..........))))))))).)))........................................................................................ |
| | .............................................................................................................................. |
| | ....................(((..((..........((.........((((((........))))))...........))..))))).... (−9.80 = −9.80 + 0.00) |
| | ..,,,{((.,{.((((.(((..........)))))))}}.}}}......................................................................................... |
| | ..................................................................................................................................... |
| | ............{{{..{{..........{{.........{{{{{\|........}\|\|\|\|}...........}}..))))}.... [−11.13] |
| | ....(((....((((.(((......)))))))...)))............................................................................................. |
| | ........................................................................................................................................... |
| | .......(((..((..........((.........((((((........))))))...........))..))))).... {−8.10 = −8.10 + −0.00 d = 17.74} |
| 54 | AUCCGGUA(N)xAAU(N)xCUAAAAA(N)xAA(N)xAUUUAUUC(N)xU(N)xA(N)xU(N)xAU(N)xUAGUAUU |
| | AAUAU(N)xUU(N)xA(N)xUU(N)xC(N)xUAUAUU(N)xAG(N)xUAUUAA(N)xUA(N)xUAG(N)xA(N)xAUA |
| | AAUUUUU(N)xUUCU(N)xUUUUG(N)xUA(N)xUGG(N)xAUU(N)xAUGGAACAA(N)xACAUAUCAAUAU |
| | GCGUGGAUAAUA |
| | ((((((((.........................((((................((..((((((.........................((((((((((((.......................... |
| | ..............................)))))..........)).)))))..((....))..........................................................))))))))))...)).... |
| | .........................(((.((........((............................................................................................... |
| | ...........................................................................................................)))).)))..))).))))).... |
| | (−13.50 = −13.50 + 0.00) |
| | ((((((((.........................,{{{................{{..{{{{{{.........................((((((((((((....................................... |
| | .................)))))..........)).)))))..,,....,,..........................................................,}}}}}}}}}...},................ |
| | .............{((.((........((............................................................................................................. |
| | .............................................................................................)))).))}..))).))))).... [−15.37] |
| | ((((((((.........................((((................((..((((((.........................((((((((((((.................................. |
| | ......................)))))..........)).)))))....................................................................))))))))))...)............. |
| | .................(((.((........((........................................................................................................... |
| | ...............................................................................................)))).)))..))).))))).... |
| | {−16.00 = −16.00 + −0.00 d = 12.62} |
| 55 | GGGCGUAUC(N)xUCUUCUUAUCUUUUCUUG(N)xUAUUUA(N)xUCUUAUGUAUCCAUUUUUAUAUU |
| | AAUUUACUACUUUUGGAAUCUAUAA(N)x |
| | ..(((((..............................................................))))).((((...........................))))............. |
| | (−2.70 = −2.70 + 0.00) |
| | ..{,{{{..............................................................))))).((((...........................))))....,,,...... [−3.89] |
| | ..(((((....................................................................((((...........................))))............. |
| | {−1.90 = −1.90 + −0.00 d = 7.68} |
| 56 | UACUUCUU(N)xUUUUCCUGGU(N)xCAGG(N)xUC(N)xACUAAA(N)xGUUAU(N)xGA(N)xAAG(N)xAU |
| | UUCGA(N)xUUUAU(N)xUU(N)xAUUU(N)xCUUAUUUAUAU(N)xA(N)xUA(N)xAUGGAUU |
| | .((((..............((((....))))..................)).))..................................................................................... |
| | ................................................................................................................ (−3.20 = −3.20 + 0.00) |
| | .{(((..............((((....))))..................)).)}........{{..,,...........,,,.....................,,,,,................................ |
| | ........................................................................................................,,}},,. [−4.85] |
| | .((((..............((((....))))..................)).))............................................................................. |
| | ........................................................................................................................ |
| | {−3.20 = −3.20 + −0.00 d = 7.76} |
| 57 | AA(N)xAAAGCCU(N)xAUG(N)xACUACGUCA(N)xGCUACAAGAAAAGACCUCAUGAUAGUCAAUAUGG |
| | GUCCUCACCACCCAUCAAUGCACGGUGUUCUUCGACUCAUUGUUACUCUAGAUGGAGAAGAUGUUA |
| | UUGACUGUGA |
| | ...................................................................................((...((...)))).................................... |
| | .............(((((((((((((((.......))))).(((((..(((......)))...)))))...((((....)))).......)))))))))).. (−22.70 = −22.70 + 0.00) |
| | ...................................................................................((...((...\|\|\|}..................................... |
| | ............,,{\|\|\|\|\|{{{((((((.......))))},,,,,,.,{{{{..{{{{{,.,,\|\|\|\|,...,,,,.,,,))})}},}...,))))))))),. [−25.50] |
| | ....................................................................................(...((...)))....................................... |
| | ..........(((((((((.(((((........)))))..................................................))))))))).. {−10.91 = −10.91 + −0.00 d = 27.35} |
| 58 | AC(N)xGACUUGUCUC(N)xA(N)xAACAAGAG(N)xAAAGA(N)xAACAAG(N)xCAAUAA(N)xAUUAUUUA |
| | UU(N)xUAUAGGUA(N)xUU(N)xCACGAU(N)xAUGUUCUCUAUCGUAACC |
| | ......((((((((...................)))..........)))))......................(((((((.....)))))))......................(((((.............. |
| | ................................................................................................................................. |
| | ......................))))).... (−6.90 = −6.90 + 0.00) |
| | ......((((({((...................)}}.....,....)))))......................{{{{{{,.....,}}\|\|},......................(((((... |
| | ....................................................................................................................................... |
| | ...........................))))).... [−8.50] |
| | ......((((((((...................)))..........)))))...............................................................(((((....... |
| | ................................................................................................................................. |
| | .............................)))))....{−6.40 = −6.40 + −0.00 d = 10.83} |
| 59 | (N)xAA(N)xGAA(N)xA(N)xAAUGCUUGAUUCA(N)xAGAC(N)xUAA(N)xU(N)xUC(N)xCCAAUAAA(N)xU |
| | C(N)xAG(N)xUUUUA(N)xGUUUUGA(N)xUCUA(N)xA(N)xAAGAAU(N)xG(N)xAG(N)xGUUUU(N)xUU |
| | U(N)xGC(N)xU(N)xUG(N)xGUG(N)xA(N)xAUA(N)xA(N)xCUACA(N)xA(N)xAUCGAA(N)xA(N)xCU(N)x |
| | A(N)x |
| | ................................................................................................................................... |
| | .....................((((.(((((......((((.(((..................................................................................... |
| | ...........))).)))))))..........................))....................))..))............................................. |
| | ...................................... (−9.10 = −9.10 + 0.00) |
| | ............................................................................................................................... |
| | .........................,{{{{({(((......((((.(((.............................................................................. |
| | ..................))).)))))))..,,.....................,\|\|,\|,..................,,..,,.,,...,,...........{{,. |
| | ...............,,..................,,,,..................... [−11.55] |
| | .................................................................................................................................. |
| | .............................(((......((((.(((........................................................................................... |
| | .....))).))))))).......................................................................................................... |
| | ............................... {−8.20 = −8.20 + −0.00d = 9.48} |
| 60 | CUGAAACAUUUUUCAAAUACAAAGGUUCA(N)xGUAUCU(N)xUCC(N)xCACGAAUUGGUGAAU(N)xU |
| | AG(N)xACAGGAUUCUUUUGUA(N)xCAGAA |
| | .(((((....))))).......((((.........................................)))).....(((......)))................................ |
| | ............................................................................................................................. |
| | ((((((...)))))).................................(−6.00 = −6.00 + 0.00) |
| | .(((({....}))))..{{{{,{((,.........................................,,,,.....{{{......}}}...................... |
| | ....................................................................................................................................... |
| | {\|{{\|\|...,})))),................................[−8.16] |
| | .(((((....))))).............................................................(((......)))....................................... |
| | .................................................................................................................................. |
| | .................................... {−4.20 = −4.20 + −0.00 d = 13.22} |
| 61 | G(N)xCG(N)xGA(N)xU(N)xAGG(N)xG(N)xAA(N)xCUAU(N)xAU(N)xUAGUGAC(N)xCUAC(N)xCC(N)xA |
| | AUUU(N)xAUUGU(N)xCAAAAUUUUC(N)xGGGAUCA(N)xAUGA(N)xUUGGACC(N)xAUGCAA(N)xAC |
| | UAG |
| | ...........................................................((.((((.......((((.........))...........)))).))))......................... |
| | .......................................................................................................................................... |
| | ..................................................................................................................................... |
| | ..............................(−6.10 = −6.10 + 0.00) |
| | ...............,...........................,...............((.(({({......((((.........))...........)))).))))..................... |
| | ....................................................................................................................................... |
| | ................................................................................................................................. |
| | ........................................ [−7.53] |
| | ...........................................................((.((((.......((((.........))...........)))).))))............................ |
| | ........................................................................................................................................ |
| | ...................................................................................................................................... |
| | ........................... {−6.10 = −6.10 + −0.00 d = 4.27} |
| 62 | AC(N)xCUAC(N)xCC(N)xAAUUU(N)xAUUGU(N)xCAAAAUUUUC(N)xGGGAUCA(N)xAUGA(N)xUUGG |
| | ACC(N)xAUGCAA(N)xACUAGAAAU(N)xACUUUU(N)xUCUUGGAUAAA |
| | ........((.((((.......((((.................................))...........)))).)))).................................................... |
| | .......................................................................................................................................... |
| | .................................................................................................................(((((((.................... |
| | .............))......)).)))..... (−8.30 = −8.30 + 0.00) |
| | ........((.(({({......((((.................................))...........)))).)))).............................................................. |
| | ......................................................................................................................................... |
| | ........................................................................................................,,(((((....................... |
| | ..........))......)))})}..... [−10.10] |
| | ........((.(((((......((((.................................))...........)))).)))).......................................................... |
| | .......................................................................................................................................... |
| | ....................... ......................................................................................(((((.................... |
| | .............))......)))........{−7.90 = −7.90 + -0.00 d = 5.25} |
| 63 | GAAACAGUUCGAAAAGGUUCAAUUGUCCGAAAUGAAUUUCUAG(N)xACUUGCGGA(N)xUUUAUC(N |
| | )xG(N)xACAUCAAGUUCGUAAAAA(N)xGAACA(N)xAAAU(N)xUUG(N)xGGGA(N)xUUC(N)xUCUGUG |
| | A(N)xGAAA(N)xAA(N)xAA(N)xU |
| | ......((((....(((((((.((.....)).)))))))................................................................................................ |
| | ....................................................................................................................................... |
| | .............................................................................(((((((..(((..............)))))))))).................)))). |
| | .................................................(((..((...)).)))......................... (−14.30 = −14.30 + 0.00) |
| | ...,{{(((,({,...,\|\|\|\|}\|\|}},{,{\|,\|\|\|}}}}}......................................................................................... |
| | ............................................................................................................................................. |
| | .............................................................................(((((((..(((..............)))))))))).................,,,}..... |
| | ...........................................,..,,...{((..((...)).))}......................... [−16.75] |
| | ....(((((.((.....))))))))............................................................................................................... |
| | ................................................................................................................................ |
| | .....................................................................(((((((..(((..............))))))))))............................... |
| | .....................................(((..((...)).)))........................... {−11.50 = −11.50 + −0.00 d = 17.77} |
| 64 | (N)xACUUGCGGA(N)xUUUAUC(N)xG(N)xACAUCAAGUUCGUAAAAA(N)xGAACA(N)xAAAU(N)xUUG |
| | (N)xGGGA(N)xUUC(N)xUCUGUGA(N)xGAAA(N)xAA(N)xAA(N)xU(N)xC(N)xA(N)xA(N)xAUU(N)xAU |
| | GAAAAGCCUUUUGCUUAU(N)xA(N)xU(N)xA(N)xU(N)xA(N)xCU(N)xC(N)xUUUUUCUACAAGAUUUC |
| | (N)xGGGAAUU |
| | ..............................................................(((((((..(((............))))))))))..................................... |
| | ...........................((..((((.((.....((((..((((................((.................................................................... |
| | ..........................)))))))))).))))))......)).... (−13.48 = −13.48 + 0.00) |
| | ..............................................................(((((((..(((............))))))))))........................... |
| | ...........,,........................{(..{(((.((,..,,((((,.((((................{,..................,,........{(((......)))}.. |
| | ....................................................}))))))).))))......}}}}})), [−16.05] |
| | ..............................................................(((((((..(((............))))))))))........................................... |
| | .........................{(((........((((..((((................(............................((((......))))................................... |
| | ....................})))))))..))))........... {−11.20 = −11.20 + −0.00 d = 16.62} |
| 65 | UG(N)xGGGA(N)xUUC(N)xUCUGUGA(N)xGAAA(N)xAA(N)xAA(N)xU(N)xC(N)xA(N)xA(N)xAUU(N)x |
| | AUGAAAAGCCUUUUGCUUAU(N)xA(N)xU(N)xA(N)xU(N)xA(N)xCU(N)xC(N)xUUUUUCUACAAGAU |
| | UUC(N)xGGGAAUUC(N)xCCGCAUUCCUAG(N)xUCAUAGU(N)xAUGUAG(N)xAUUGAAAA(N)xGA(N) |
| | xA(N)xG(N)xACUCUUC(N)x |
| | ...((((.(((..((((((..((((.......................((..................................................................................... |
| | ..))))))))))...........))))).))...........))((((..(((.....))).....))))................((..((..........))))... (−15.18 = −15.18 + 0.00) |
| | ...((((.(((..((((((,.((((.......................{,.....................,,.....{(((.....)))}............................................ |
| | ....})))))}))).........))))),,)...........))((((..(((.....))).....))))................{(..({..........)))}... [−17.38] |
| | ...(((..(((..((((((..((((.......................(.............................((((.....)))).......................................... |
| | ......)))))))))..........)))))..)...........))((((..(((.....))).....))))................((..((..........))))... |
| | {−13.20 = −13.20 + −0.00 d = 15.04} |
| 66 | U(N)xC(N)xA(N)xA(N)xAUU(N)xAUGAAAAGCCUUUUGCUUAU(N)xA(N)xU(N)xA(N)xU(N)xA(N)xCU( |
| | N)xC(N)xUUUUUCUACAAGAUUUC(N)xGGGAAUUC(N)xCCGCAUUCCUAG(N)xUCAUAGU(N)xAUGU |
| | AG(N)xAUUGAAAA(N)xGA(N)xA(N)xG(N)xACUCUUC(N)xGACUUUACCCCUUCUUUU(N)xCUU(N)xU |
| | C(N)xUGUG(N)xUU(N)x |
| | ............(((.....((((.((((.....))))................................................................((((((........ |
| | ........))))))...)))))))......((((...........(((((...................)))..........))..))))......................................... |
| | ........... (-11.10 = -11.10 + 0.00) |
| | ............,,{.....{{({.((((.....)))).............................................{{,,,.\|\|,..........((((((.................)))))).,.))} |
| | }}}........,\|\|\|\|((((,.{..((........................)))))...........))...,,,.............................................. [-14.37] |
| | ..............(.....(((..((((.....))))................................................................((((((................)))))).. |
| | ..)))).......................((((..(..((...........)))))..........)).......................................................... |
| | {-7.50 = -7.50 + -0.00 d = 14.36} |
| 67 | UACUGCGGUAUUUAUGUUAAUGCACUUCCCAAUGAUACGUAAACAAGGCAUUUCUGGUCCUUUAU |
| | AG(N)xAAAAGA(N)xUCU(N)xAUCAUAGA(N)xUA(N)xU(N)xUU(N)xG(N)xU(N)xAAUCUAUCAUUUAU |
| | CGCUUGGGGGA(N)x |
| | .......(((((......)))))..{(((((((((((..................((((.(((......................................................................... |
| | ........................................................................................................................................ |
| | ......))).))..)).....(((((..................................)))))....))))).)))))))....... (-14.60 = -14.60 + 0.00) |
| | ...,,,,{{({,......},))),.{(((((((((((.,,....,{({,......{\|\|\|}\|\|,,,,,..................................................................... |
| | ......................................................................................................................................... |
| | .........,,.},..,),.,,.{((((................................})))).,,.))))).))))))}....... [-18.23] |
| | .........................{(((((((((((..................................................................................................... |
| | .......................................................................................................................................... |
| | ............(((((.......................................)))))....))))).)))))))....... {-7.94 = -7.94 + -0.00 d = 17.52} |
| 68 | GAAAAAUUA(N)xUC(N)xAUGUCCGGU(N)xUCCUUCGGGGGAUGG(N)xAUCUAUAAAAAUUCACCUA |
| | UCCCAAUAACAAAAAAACCUGACCU |
| | ........................................................................................................................................ |
| | .................................................................................................((((.........))))(((((((................ |
| | ..)))))))..................... (-13.10 = -13.10 + 0.00) |
| | .............................................................................................................................. |
| | .........................................................................................................,,({((,........}}))(((((((......... |
| | .........)))))))............,,,.,,,.. [-13.90] |
| | .................................................................................................................................... |
| | ......................................................................................................(((..........)))(((((((.............. |
| | ....))))))).................... {-11.80 = -11.80 + -0.00 d = 6.14} |
| 69 | (N)xAUGUCCGGU(N)xUCCUUCGGGGGAUGG(N)xAUCUAUAAAAAUUCACCUAUCCCAAUAACAAAA |
| | AAACCUGACCUGAAUGAUCCUGUAUUAAGAGCUAAAUUGGCUAAAGGUAUGG |
| | ....................................................................................................................................... |
| | .....((((........))))(((((((...................)))))))............((..((((........((......))(((((...)))))..))))..)) (-21.30 = -21.30 + 0.00) |
| | ........................................................................................................................................ |
| | ....((((........))))(((((((...................)))))))............((..((((........{{......,,(((((...)))))..))))..)) [-22.64] |
| | ................................................................................................................................. |
| | ...........((((........))))(((((((...................)))))))............((..((((........((........(((((...)))))..))))..)) |
| | {-21.10 = -21.10 + -0.00 d = 7.62} |
| 70 | UCCAGUAGCGACAACGGUCUUUUUGAUUGGUACCGCAGUGGCCCUUUGGUUGGGUAUUGGUGCAA |
| | CAUUACCUAUUGAUAAAUCCCUAACUUUAGGUCUUUUUUAA(N)xUUUUGAAAUUGAUU |
| | .((((..((.((..((((((........)).))))..)).))...)))).((((((.((......)).))))))........((((...))))((..((((((.................................... |
| | ...............................................................................................(((.............................................. |
| | .....................................................................................................(((............................ |
| | ..........................................))))))..)).. (-25.80 = -25.80 + 0.00) |
| | .{{{,..((.((..((((((........)).))))..)).)),,,,,\|\|,((((((,((,,....}}.))))}}..,..,,.\|\|\|\|,,,.,))((..(((((,.................................. |
| | .................................................................................................(((........................................ |
| | ...........................................................................................................(((.............................. |
| | .........................................)))))..)).. [-27.76] |
| | ..(....((.((..((((((..........)).))))..)).)).....)...(((((.((......)).))))).............................((..(((((........................ |
| | .................................................................................................(((.......................... |
| | .........................................................................................................................(((.. |
| | .....................................................................)))))..)).. {-17.00 = -17.00 + -0.00 d = 20.60} |
| 71 | GAAAUAAGAC(N)xGAUGUGCGUA(N)xUCUA(N)xGG(N)xGAAUAG(N)xU(N)xCGCUU(N)xCA(N)xAAG |
| | (N)xU(N)xGAAUU(N)xCU(N)xCC(N)xC(N)xU(N)xA(N)xGAUA(N)xC(N)xACC(N)xUAU(N)xU(N)xCAAU |
| | UGA(N)xAU(N)xUCC(N)xGGAU(N)xCU(N)xA(N)xU |
| | ..............(((..........(((...............))).........)))...................................................................... |
| | ....................(-3.60 = -3.60 + 0.00) |
| | ...........,,.(((..........(((...............))).........)))...,.................................................................. |
| | ....................[-4.50] |
| | ..............(((..........(((...............))).........)))..................................................................... |
| | .....................{-3.60 = -3.60 + -0.00 d = 3.47} |
| 72 | U(N)xGGUC(N)xAUUGA(N)xG(N)xAU(N)xUCAAUG(N)xGUAA(N)xUU(N)xAGGAUUAA(N)xUAUUUA |
| | GGUAU(N)xA(N)xGAUAUUACCU(N)xCUUUUUUUCCCCUUU(N)xCAAAC(N)xAA(N)xAUUGAA(N)xA |
| | ..............(((((........)))))................................................(((..((........((((...............)))))........ |
| | ))..)))...((........(((...........))))).......................................................................... (-8.90 = -8.90 + 0.00) |
| | ..............(((((........}))))................................................(((..((........((((...............)))))........ |
| | }}..)))...((........(((...........))))).......................................................................... [-10.14] |
| | ..............(((((........)))))................................................(((..((........((((...............)))))........))..)))... |
| | ((........(((...........))))).......................................................................... {-8.90 = -8.90 + -0.00 d = 4.55} |
| 73 | CGUGGCGAUCAGUUGGACCUUUGA(N)xU(N)xUAAUUAACAUCUCUUUUUUUGAUUG(N)xACCUC(N |
| | )xC(N)xUC(N)xC(N)xUUUC(N)xUU(N)xUAA(N)xU(N)xACACAG(N)xGAGGUC(N)x |
| | .....(((((((..(((...((((..................................................................................................................... |
| | .............................................................................................................................................. |
| | ................))))....)))....))))))).....(((((................................................................................)))))... |
| | (-8.90 = -8.90 + 0.00) |
| | ...{{(((((((..(((...{((,.................................................................................................................... |
| | ............................................................................................................................................ |
| | ..................,}}}....)))....))))))).....\|\|,,........................................................,{{...{{..........}}...... |
| | )))).... [-10.38] |
| | ...(((((((((..(((...(((................................................................................................................... |
| | ...................................................................................................................................... |
| | ...........................)}}....)))....))))))).....))...........................................................((...((..........))...... |
| | ))...... {-7.20 = -7.20 + -0.00 d = 8.63} |
| 74 | UUAACAUCUCUUUUUUUGAUUG(N)xACCUC(N)xC(N)xUC(N)xC(N)xUUUC(N)xUU(N)xUAA(N)xU(N |
| | )xACACAG(N)xGAGGUC(N)xAAAUUC(N)xAGAUUGCCGCU(N)xC(N)xAAGUUAGUGAAGCU(N)xG(N)x |
| | UUUCAGUC |
| | ..........(((((........(((((...................................................................................))))).......... |
| | )).)))..((((((....................................))))..)).......... (-8.60 = -8.60 + 0.00) |
| | ..........((((({{......(((((...................................................................................)))))......,)}., |
| | }.)))..{,((((....................................))))..}}.......... [-10.60] |
| | .............((........(((((...................................................................................))))).......... |
| | )).)))..((((((....................................)))).............. {-4.10 = -4.10 + -0.00 d = 9.27} |
| 75 | AGAAAAGAUAAGACUGU(N)xAAC(N)xGAAAGGAUUCUUUUUGUAACCCAAU(N)xA(N)xCA(N)xUCU |
| | UG(N)xUAUG(N)xACUAUA(N)xU(N)xAGUAGG(N)xAUAA(N)xA(N)xAAUAGU(N)xA(N)xUGAUAAAAA |
| | (N)xUG(N)xAA(N)xA(N)xGAA(N)xAAGAUUAUA(N)xG(N)xA |
| | ....((((.......((...((..........((((((...)))))))).)).......)))).......(((((........................................................ |
| | ....................)))))........................................................................... (-7.00 = -7.00 + 0.00) |
| | ....,{{{.......((...((..........((((((...)))))))).}}.......}}}}.......,((({..................,,,,.................................... |
| | ..................}}}}}........................................................................... [-8.30] |
| | ...............((...((..........((((((...)))))))).))................................................................................. |
| | .................................................................................................. {-5.10 = -5.10 + -0.00 d = 9.88} |
| 76 | GUUAC(N)xAC(N)xAA(N)xCC(N)xCGGCUCUUUUU(N)xA(N)xU(N)xUAGUUUAUAAG(N)xU(N)xUUAU |
| | C(N)xCAAA(N)xA(N)xG(N)xUU(N)xUA(N)xU(N)xC(N)xCAAAAGUUA(N)xCUCAA(N)xUGAAU(N)xUCG |
| | (N)xUUGA(N)xUUGGAAUCG(N)xCGGGAG(N)xG(N)x |
| | .................((.....(((((.................................................................)))............................ |
| | ...............................................((((.......(((......)))....))))...................)))).......... (-8.20 = -8.20 + 0.00) |
| | .................((.....((..,,,..............................................................,,....,......,......................... |
| | ........................................((((.......(((......)))....))))........,}},.......)))).......... [-10.55] |
| | .................((.....((................................................................................................ |
| | ..................................................((((.......(((......)))....))))...................)))).......... |
| | {-7.58 = -7.58 + -0.00 d = 6.75} |
| 77 | CG(N)xCGGGAG(N)xG(N)xGG(N)xUAG(N)xAUGUC(N)xA(N)xCAGAUGAUGAAUCAAUUUCU(N)xUU |
| | UU(N)xAUAC(N)xAC(N)xCUGCCAC(N)xUU(N)xU(N)xAUCUUUAUUAGUGC(N)xU(N)xG(N)xU(N)xC(N |
| | )xU(N)xAUAAUG |
| | ..................................((.....(((......((.........(((.((..........))..)))........)).......))))).............................. |
| | ...................................................................................................... (-5.90 = -5.90 + 0.00) |
| | ..................................((.....(((......((.........{((.((.,,,.....}))..,,,........)}.......)))))...,.......................... |
| | ...................................................................................................... [7.58] |
| | ..................................((.....(((......((........................................)).......)))))............................. |
| | ....................................................................................................... {-3.50 = -3.50 + -0.00 d = 8.62} |
| 78 | UUU(N)xCAAUUGAACUUAUUCUUUGU(N)xAUUGGUAU(N)xUUUUGCUUAUCUCUU(N)xAU(N)xUU |
| | UG(N)xCAAA(N)xACU(N)xUA(N)xGGUAAGUGCCUU(N)xU(N)xUGAUAA(N)xACA(N)xU(N)xAUGUA( |
| | N)xUC(N)x |
| | .........................................................................................(((((.......(((((((......................... |
| | ............................................................)).))))))))))................................ (-8.00 = -8.00 + 0.00) |
| | ..................,,......,,...,.........................................................(((((....,.,(((((((.......................... |
| | ...........................................................)).))))))))))..........,,,...........,...,... [-9.67] |
| | .........................................................................................(((((.......(((((((........................ |
| | .............................................................)).)))).)))))................................ |
| | {-7.20 = -7.20 + -0.00 d = 5.93} |
| 79 | CUUAUCUCUU(N)xAU(N)xUUUG(N)xCAAA(N)xACU(N)xUA(N)xGGUAAGUGCCUU(N)xU(N)xUGAU |
| | AA(N)xACA(N)xU(N)xAUGUA(N)xUC(N)xAAA(N)xAGA(N)xAU(N)xA(N)xU(N)xAUUU(N)xCAUU(N)x |
| | UAG |
| | ((((((.............................................................................))))))...........((((............))..))........ |
| | ................................................. (-2.70 = -2.70 + 0.00) |
| | ..,,,,.............................................................................\|\|\|}}}....,......((((............))..))..... |
| | ...................,................................ [-3.99] |
| | ....................................................................................................((((............))..)).......... |
| | ............................................... {-1.10 = -1.10 + -0.00 d = 7.24} |
| 80 | CAAA(N)xACU(N)xUA(N)xGGUAAGUGCCUU(N)xU(N)xUGAUAA(N)xACA(N)xU(N)xAUGUA(N)xUC( |
| | N)xAAA(N)xAGA(N)xAU(N)xA(N)xU(N)xAUUU(N)xCAUU(N)xUAGCUUCUUC(N)xAUGCCUACU |
| | ....................................(((....))).......((((....................))..)).....................................((............ |
| | ............................................................................................................................... |
| | ................)).....(-3.60 = -3.60 + 0.00) |
| | ....................................{{.(((\|.....,....((((....................))..))..............,......................{,......... |
| | ........................................................................((((....................))..))............................... |
| | ............,))}}}.. [-5.13] |
| | .....................................................((((....................))..))................................................ |
| | ...................{-1.10 = -1.10 + -0.00 d = 8.57} |
| 81 | UAUUUAA(N)xA(N)xAU(N)xU(N)xCAUAUUUGAA(N)xAU(N)xGC(N)xACA(N)xA(N)xUUGAUAAAAA( |
| | N)xUCAAUU(N)xU(N)xUUCUGUG(N)xAGAUUCCCUG(N)xUAUUCUAUAAUAAUGG(N)xAC(N)xUAUG |
| | UCA(N)xAUUGACAA(N)xUU |
| | ......................................................................................................................................... |
| | ..............................................................................((((.......)))).......................((....................... |
| | ((((....))))..))..........................(((((...............))))).... (-5.20 = -5.20 + 0.00) |
| | .......................................................................................................................................... |
| | .............................................................................,{{{.......}}},..................,....((.............. |
| | .........((({....})))..))..........................(((({...............})))).... [-7.44] |
| | ......................................................................................................................................... |
| | ....................................................................................................................((....................... |
| | ((({....})))..))..........................(((((...............))))).... {-5.00 = -5.00 + -0.00 d = 8.22} |
| 82 | GGAU(N)xGC(N)xUGU(N)xUGU(N)xU(N)xCCAU(N)xUCAUU(N)xG(N)xGG(N)xAUGAUUUG(N)xU(N) |
| | xUU(N)xAU(N)xG(N)xC(N)xG(N)xG(N)xUUU(N)xACU(N)xUCUACG(N)xCAUCUG(N)xAUCU(N)x |
| | ((...((......................))...))..............................................((.(((............................................ |
| | .............................................)))))............................. (-3.60 = -3.60 + 0.00) |
| | ((...{(......................))...}\|,,...............................,,...........\|\|.\|\|\|........................................ |
| | ...................................................))...))............................. [-4.35] |
| | ((...{(......................))...}).............................................................................................. |
| | ................................................................................. {-1.80 = -1.80 + -0.00 d = 7.11} |
| 83 | UAGGAUUCAA(N)xG(N)xGAAAGAUUCUAAUGGG(N)xG(N)xAAGGGUUAU(N)xCAAGAC(N)xUU(N)x |
| | C(N)xU(N)xCC(N)xAU(N)xUCCUUUCUACUG(N)xC(N)xUA(N)xA(N)xUC(N)xU(N)xUU(N)xCUACUACU |
| | A(N)xA(N)xU(N)xG(N)xU(N)xAG(N)xU(N)xGUC(N)xUACUGA(N)xCUGGGUCU(N)xUGAA(N)xUUCCA |
| | UU |
| | (((((((..........))))))).................................(((.......((((((.............((......................................... |
| | .......................................................................................................)))))).............................. |
| | ....................))....)))... (-11.07 = -11.07 + 0.00) |
| | (((((((..........))))))).................................(((.......((((((.............(,................................................ |
| | ............................................................................,...................)))))).................................... |
| | ..............))....}))...[-12.42] |
| | (((((((..........))))))).................................(((.......((((((.............(.................................................... |
| | .............................................................................................))))))........................................ |
| | ..........))....)))...{-10.73 = -10.73 + -0.00 d=7.57} |
| 84 | C(N)xU(N)xUU(N)xCUACUACUA(N)xA(N)xU(N)xG(N)xU(N)xAG(N)xU(N)xGUC(N)xUACUGA(N)xCU |
| | GGGUCU(N)xUGAA(N)xUUCCAUUG(N)xGAU(N)xAACC(N)xGGAUA(N)xGGA(N)xAAUAA(N)xA(N)xA |
| | UUCC(N)xAUUUAUUCC(N)xUACAUGUUCCAUU(N)xAGU(N)xA(N)xAGAGUUCCUUGAG(N)xA(N)xU |
| | AU(N)xCAUUGCAUAUUUGA(N)xC(N)xUU(N)xUUAUUUAGUCUUUCCAGAU(N)xU(N)xACAAA(N)xU( |
| | N)xCAU(N)x |
| | ......................................................................((................((((((........................................ |
| | (((.(((.((((..((.......................((............((.....(((..((((..................................................................... |
| | .............))))))).............))...))))))......)).......................)))))).................................................... |
| | ...................((....))...............))))))))........................................................ (-13.70 = -13.70 + 0.00) |
| | ......................................................................{{................((((({,,.................................... |
| | .,(((.(((.((((..((.......................((............((.....(({.,((((.................................................................. |
| | ................))))))).............))...))))))......)).......................))))))...\|,,.......................................... |
| | .......................,{....}}................}}}))))}....,}.................................................. [-16.81] |
| | ......................................................................((................((((((................................. |
| | .......(((.(((.((((..((.......................((............((.....(((..((((................................................... |
| | ...............................))))))).............))...))))))......)).......................))))))................................... |
| | ............................................................)))))).......................................................... |
| | {-11.01 = -11.01 + -0.00 d = 17.61} |
| 85 | (N)xAG(N)xU(N)xGUC(N)xUACUGA(N)xCUGGGUCU(N)xUGAA(N)xUUCCAUUG(N)xGAU(N)xAACC( |
| | N)xGGAUA(N)xGGA(N)xAAUAA(N)xA(N)xAUUCC(N)xAUUUAUUCC(N)xUACAUGUUCCAUU(N)xAG |
| | U(N)xA(N)xAGAGUUCCUUGAG(N)xA(N)xUAU(N)xCAUUGCAUAUUUGA(N)xC(N)xUU(N)xUUAUUU |
| | AGUCUUUCCAGAU(N)xU(N)xACAAA(N)xU(N)xCAU(N)xA(N)xC(N)xAUAUAGGAAUUG(N)xAUUAG( |
| | N)xAAGUGGAUUUGUUGGAU(N)xUGGUUCAU(N)xCA(N)xAUA(N)xGUGUUCGGUCAGAA(N)xUCC(N |
| | )xCUUUUU(N)xGAC(N)xU(N)xCUGCGCCAUU |
| | .............................(((..((((.........((((.........................)))).((..((............................................. |
| | .......((............((.....(((..((((......................................................)))))))...............))...))))))........ |
| | .................................................((((((((..........................................((....)).................... |
| | ((((....))))....................................................................................)))))))........................... |
| | .....................................................................))))))).((.(((((.......)))))......)).................( |
| | (((...(((((((..................))))......))).....)))).... (-33.20 = -33.20 + 0.00) |
| | .............................(((..((((.........,(((.........................)))).((..((........................................... |
| | .........((............((.....(((..((((......................................................)))))))...............))...))))))............ |
| | .............................................((((((((..........................................((....))....................((((.. |
| | ..))))....................................................................................))))))).............................. |
| | ..................................................................))))))).((.(((((.......)))))......)).................((((...(((((((.... |
| | ..............))))......))).....)))).... [-36.98] |
| | .............................(((..((((.........((((.........................)))).((..((.................................................... |
| | ((............((.....(((..((((......................................................)))))))...............))...))))))..................... |
| | ....................................((((((((..........................................((....))....................((((....))))...... |
| | ..............................................................................)))))))......................................... |
| | .......................................................))))))).((.(((((.......)))))......)).................((((...(((((((......... |
| | .........))))......))).....)))).... {-31.70 = -31.70 |
| | + -0.00 d = 20.57} |
| 86 | U(N)xUCCAU(N)xAUC(N)xA(N)xUAAU(N)xU(N)xGC(N)xUC(N)xUAGUG(N)xAA(N)xU(N)xAACUAAA( |
| | N)xGAAA(N)xAUAA(N)xA(N)xUAGAUGGGAGAUAGAAAAAUAGAAGA(N)xGAAACA(N)xAACUAAUU |
| | AU(N)xAU(N)xAAA(N)xUAUC(N)xUCUCAG(N)x |
| | ......................................................((((............))))...............................(((((((((.................. |
| | .......................................................))))....)))))............................................. (-10.57 = -10.57 + 0.00) |
| | ......................................................((((............))))...............................(((((((((...................... |
| | ..........................,....,)))................))))....)))))............................................. [-11.69] |
| | ......................................................((((............))))...............................(((((((((....... |
| | ...............................................)))................))))....)))))............................................. |
| | {-10.10 = -10.10 + -0.00d = 3.50} |
| 87 | AUUAU(N)xAU(N)xAAA(N)xUAUC(N)xUCUCAG(N)xAGAUUCAUUAAUGUUG(N)xGCGGG(N)xUUU( |
| | N)xCUUUGUAUGU(N)xGUUGU(N)xCCG(N)xGAA(N)xAGAG(N)xGAGGACUCA(N)xAUGAUUAUUCG |
| | U(N)xUCGCCGGAACCAGAA |
| | .................................................................................................................................. |
| | .......................(((....((((...........................((...((........................))))...................................... |
| | ..............................................((((....))))...................))))))).......... (-12.60 = -12.60 + 0.00) |
| | ................................................................................................................................... |
| | ......................(((....((((...........................((...((........................)))).................................... |
| | ................................................((((....))))...................})))))}.......... [-14.16] |
| | .......................................................................................................................................... |
| | ...............(((....((((...........................((...((........................))))................................................ |
| | ....................................((((....))))...................))))))).......... {-12.30 = -12.30 + -0.00 d = 7.64} |
| 88 | GGCUAGGAGGAUUUGAAAGACA(N)xCU(N)xAUGGCAUUAAGAUUUCCAAGGUUUAGCCAAGGCUU |
| | AGCCCAGGACCCCACUAC |
| | .((((............................................................................................)))).......... |
| | (((..((((.(((....))).))))..))).........(-13.60 = -13.60 + 0.00) |
| | .,,{{(,.((.,{{......{,............................................................................}).......}}.,{( |
| | (..((((.(((....))).))))..))}.,}..,,.. [-14.92] |
| | ...............................................................................................................(((..( |
| | (((.(((....))).))))..))).........{-11.70 = -11.70 + -0.00 d = 11.70} |
| 89 | AUUUACUUAUGCAGCUUUCUUGAUUGCCUCUACAUCAGGCAAAUUUGGUUAA(N)xU(N)xUUU(N)x |
| | U(N)xUGAAUGUCU(N)xUGU(N)xAUCCGCG(N)xAGAA(N)xUCUCAUUUC(N)xUUUC(N)x |
| | ..................................................((((((........))))))...................................................... |
| | ...................................................................(((...(((.(((.....)))..........))).......))) (-12.80 = -12.80 + 0.00) |
| | .......................................,,.,..,,.,,((((((........))))))......................................................... |
| | ................................................................(((...(((.(((.....)))..........))).......))) [-14.56] |
| | ..................................................((((((........))))))............................................................ |
| | .............................................................(((...(((.(((.....)))..........))).......))) {-12.80 = -12.80 + -0.00 d = 9.35} |
| 90 | UCUCAAU(N)xAU(N)xAUG(N)xA(N)xU(N)xUCAUUUAUAAGU(N)xUUACUAG(N)xA(N)xUCGAA(N)xA |
| | AUUUAUG(N)xACGUUCA(N)xA(N)xAAAUG(N)xUAUAGAUCCA(N)xAUGUCAC |
| | .........................................................................................................................(((((((..((((... |
| | ...................))))..........)))))))............................................................................................ |
| | ...............................(-3.60 = -3.60 + 0.00) |
| | .........................................................................................................................(((((((..((((.. |
| | ....................))))..........)))))))..................................................................................................... |
| | ......................[-4.32] |
| | .........................................................................................................................(((((((..((((. |
| | .....................))))..........)))))))................................................................................................. |
| | ..........................{-3.60 = -3.60 + -0.00 d = 4.40} |
| 91 | (N)xA(N)xAAAUG(N)xUAUAGAUCCA(N)xAUGUCACAUUCAGUAAAGAUUUAUGAU(N)xACAUGUAU |
| | AGGGUGU(N)xACUCAAUGUGUCCGAGCCUG |
| | ............................(((((((........................................................................................... |
| | ......................................................................................)))))))(((...............((((....((.... |
| | ..................................................))..))))))).......... (-8.50 = -8.50 + 0.00) |
| | ............................(((((((.................................................................................................. |
| | ...............................................................................)))))))(((...............((((....((....................... |
| | ...............................))..))))))).......... [-9.30] |
| | ............................(((((((....................................................................................................... |
| | ..........................................................................)))))))(((...............((((....((.................... |
| | ..................................))..))))))).......... {-8.50 = -8.50 + -0.00 d = 5.93} |
| 92 | GUUUAUUUAUGGCAU(N)xGAAACAACUCGCAGUAUGGGUCUAGCUUAUUGA(N)xUAUGUUCC(N)x |
| | AAAAA(N)xACUCCAAU(N)xGGA(N)xAU(N)xCCAU(N)x |
| | ..........(((.....((..(((((...))).))..))..)))........................................................................................ |
| | .................................................(((((...........................))).)).................................................. |
| | ............. (-7.60 = -7.60 + 0.00) |
| | ..........(((.....((..(((((...))).))..))..)))............................................................................................ |
| | .............................................(((((...........................))).)).................................. |
| | ............................. [-8.69] |
| | ..........(((.....((..(((((...))).))..))..)))............................................................................................. |
| | ............................................(((((...........................))).))................................................. |
| | .............. {-7.60 = -7.60 + -0.00 d = 7.05} |
| 93 | AU(N)xUU(N)xUUUUUU(N)xGG(N)xUUUUAAGAAA(N)xA(N)xAGAAA(N)xAU(N)xC(N)xU(N)xUA(N)x |
| | UUUA(N)xCCGGAUC(N)xAUUGUACCCUUCA(N)xAUUUG(N)xAAUUAGGAAU(N)xUCCGUGGACA(N) |
| | xACUAUAACCAUAAGCGG(N)x |
| | .....((((((..((...))))))))...............................................(((.................................((((...........) |
| | )...........)).................(((....................)))((((..........))))...))).. (-5.10 = -5.10 + 0.00) |
| | .....((((((..((...))))))))...............................................(((.................................((((...........)).. |
| | .........))............),...\|\|,....................,))((((..........))))...))).. [-8.44] |
| | .......................................(((...................................................................((.............((.. |
| | ..........).........)....((........................)).((((..........))))...))).. {-1.30 = -1.30 + -0.00 d = 17.09} |
| 94 | AAGGA(N)xAU(N)xAAC(N)xG(N)xAAGA(N)xUUAGAGCUAUAAUU(N)xAAA(N)xUCUC(N)xU(N)xGUG |
| | (N)xAUUU(N)xUCUCUCCCUCU(N)xUUUCUCUUUUUUC(N)xC(N)xCUU(N)xUUUGUUUGUUUAG(N)x |
| | A(N)xAUAAGGGAG(N)xGAA(N)xA(N)xGAGAAA(N)xGAAU |
| | ...((.((..((.........(((.........((.((.........)).))).........))...............))))...............(((....((((((((((..................... |
| | .......................................................................................................))))))).)))....)))............ |
| | ............ (-13.60 = -13.60 + 0.00) |
| | .,(((.((..((.........(((.........((.((.........)).))).........))...............))))...............(((....((((((((((.................... |
| | ............... ...................................................................................... |
| | ...))))))).)))....)))........................ [-15.50] |
| | ...((.((..((.........(((.........((.((.........)).))).........))...............))))...............(((....((((((((((................ |
| | ............................................................................................................))))))).) |
| | ))....)))........................ {-12.30 = -12.30 + -0.00 d = 6.50} |
| 95 | (N)xAUUU(N)xUCUCUCCCUCU(N)xUUUCUCUUUUUUC(N)xC(N)xCUU(N)xUUUGUUUGUUUAG(N)x |
| | A(N)xAUAAGGGAG(N)xGAA(N)xA(N)xGAGAAA(N)xGAAUAAA(N)xAGUGGAU(N)xU(N)xCGCCAACU |
| | GC(N)xGAG(N)xACUCG(N)xAAUU |
| | .......................(((...(((....((((((((((............................................................................................ |
| | ...............................))))))).))).....))).............(((((..............)).))).........)))............................. |
| | ........ (-13.50 = -13.50 + 0.00) |
| | .......................,((,,.(((....((((((((((.......................................................................................... |
| | .................................))))))).))).....))).............(((((..............)),,)).........))).............................. |
| | ....... [-15.06] |
| | .............................(((....((((((((((....................................................................................... |
| | ....................................))))))).))).....))).............(((((..............)).)))...................................... |
| | ........... {-9.90 = -9.90 + -0.00 d = 7.91} |
| 96 | (N)xAU(N)xA(N)xAAA(N)xA(N)xU(N)xUUAAAACAUUU(N)xAUCUAUAUA(N)xCUCCAUAUGUAUAUA |
| | GUAA(N)xAUAACUAU(N)xAGUUAGUA(N)xAUAAC(N)xAGGAUAUU(N)xAC(N)xU(N)xCGCUUU(N)xA |
| | AAAAUA(N)xUUU |
| | .........................................................................((((.(.(((((((....................))))))).))...)).( |
| | (((.....))))....................................................(-4.60 = -4.60 + 0.00) |
| | .........................................................................((((.(.(((((((....................))))))))))...)). |
| | {{{{.....\|\|},..,,,,.,{{{{{,.........................,}}}.....},. [-8.00] |
| | .........................................................................((((.(.(((((((....................))))))))))...)).)).. |
| | .................(((............................)).....).. {-0.50 = -0.50 + -0.00 d = 10.04} |
| 97 | (N)xC(N)xCUGAGAAG(N)xAAUUAAA(N)xAUU(N)xGAUCUU(N)xA(N)xAAUUAUUAUU(N)xAUGGUU |
| | GCAACUUUAGAA(N)x |
| | .............................((((...(((........)))...))))........................((((((........................................ |
| | .....................................))))))................. (-2.30 = -2.30 + 0.00) |
| | .............................,,((((((.,........)).)))))))........................((((((................................................ |
| | .............................))))))........,,,...... [-3.47] |
| | .....................................((.........))...))))........................((((((.............................................. |
| | ...............................))))))................. {2.90 = 2.90 + -0.00 d = 9.89} |
| 98 | AA(N)xAUU(N)xGAUCUU(N)xA(N)xAAUUAUUAUU(N)xAUGGUUGCAACUUUAGAA(N)xAGACGC(N) |
| | xGAAGAAAAAAGAGAUUGGGGAACAUUU |
| | .........((((((...........((((((.................................................................................)))))) |
| | ((..((........................))..))...........))))))........... (-4.40 = -4.40 + 0.00) |
| | .......{.((((((...........{(((((.................................................................................))))))( |
| | (..((.........................))..))..,........))))))}......... [-5.80] |
| | .......(.((((((...........((((((.................................................................................))))))( |
| | (..((........................))..))...........))))))........... {-4.30 = -4.30 + -0.00 d = 4.75} |
| 99 | GAUUUAGCAUCUAACGAAAUUCUUCCAGUUGCGAUUUCUGCACCUUCUGUUGUAGGUUAA(N)xUC |
| | AUU(N)xAUU(N)xA(N)xAAUUCAA(N)xUCAAGGAUUG(N)xAAAAA(N)xUUA(N)xAAU(N)xUA(N)xAUA |
| | UUA(N)xUU(N)xUAACUU(N)xA(N)xACU(N)xUA(N)xUA(N)xU(N)xUC(N)xU(N)xAUA(N)xGA(N)xUCU |
| | U(N)xAUUCUA(N)xU(N)xA |
| | (((((((((.((...(((....))).)).)))....((((((..........))))))........................................................................ |
| | ..........(((((...((...))))).)).........................................................................................))..)).))..... |
| | ................ (-14.60 = -14.60 + 0.00) |
| | (((((((((.((...(((....))).)).)))...,((((((,.........)))))).................................................................... |
| | .............(((((...((....))))).))...............,....,................................................................... |
| | }}..)).}}...................... [-16.80] |
| | (((((((((.((...(((....))).)).)))....((((((..........))))))........................................................................ |
| | ..........(((((...((...))))).)).........................................................................................))..)).))..... |
| | ................ {-14.50 = -14.50 + -0.00 d = 13.54} |
| 100 | GA(N)xAUCA(N)xAACAAA(N)xU(N)xAU(N)xGAA(N)xU(N)xCC(N)xU(N)xU(N)xGCU(N)xCG(N)xGAAA |
| | UAA(N)xUCC(N)xA(N)xC(N)xUG(N)xAACAGGGGAG(N)xGUCCA(N)xUAACAUAGUCAUAG(N)xUCUA |
| | GCCUU(N)xUUC(N)xCAAUGCA(N)xAUA |
| | .................................................................................................((......(((........((.................. |
| | .............................))(((...................................................................)))............................ |
| | .........(((.......)))........)))..............))............. (-2.10 = -2.10 + 0.00) |
| | ................................................................................................................,(......,((........((... |
| | .............................))(((...................................................................))).................................... |
| | .,,........,,,.,......,................),............. [-4.11] |
| | ................................................................................................................................. |
| | ..((................................))(((...................................................................))................................... |
| | ........................................................ {-1.40 = -1.40 + -0.00 d = 7.47} |
| 101 | UGGAGAUCCAACUACAAGAAGACAGGUAGUCUGA(N)xU(N)xACGACAUUGC(N)xUUUGGUCUCU(N) |
| | xUUCGAC(N)xUCUAU(N)xGAU(N)xUU(N)xAUU(N)xUUUGUG(N)xAUUG |
| | .(((((.(((..............((((((((......................................................................................................... |
| | ...............................................................)))..)))...))))))))))......................................................... |
| | ................................................(-10.10 = -10.10 + 0.00) |
| | ,((....}\|\|..{{{{((({{,,{{(((((((................................................................................................ |
| | ........................................................................)))..)))...)},}\|\|\|},..........,,,.......,......................... |
| | ..........................................,}.}))))}........ [-12.62] |
| | .((....))...(((((((.....((((((((.......................................................................................................... |
| | ..............................................................)))..)))...)................................................................ |
| | ........................... ).)))))) .... ... {-4.04 = -4.04 + -0.00 d = .13.27} |
| 102 | AUUGCUGUUUUUGUUUCUGUAUUCCUGAUUUAUCCACUAGGUCAGUCUGGUUGGUUCUUUGCAC |
| | CUAGUUUUGGUGU |
| | ..........................((((((((.....)))))))).................(((((........))))) (-11.90 = -11.90 + 0.00) |
| | ..............................,,...,.({(((((\|....,)))),.))......(((((.......))))) [-13.45] |
| | ........................................((((.....))))...........(((((.......))))) {-8.30 = -8.30 + -0.00 d = 8.33} |
| 103 | CUUU(N)xUUUCAAGGAUUUCA(N)xUAAUUGGAC(N)xGU(N)xUGAACCCAUUUC(N)xAUAUG(N)xAU |
| | GGGAGUUGCCGGUGUAU(N)xUGGGCGCUGC(N)xUU |
| | .......((((.....((((...............))))...............))))(((((...............)))))(((.(((..................... |
| | .......))))))......... (-12.40 = -12.40 + 0.00) |
| | .......,{{{.....((((...............))))...............}}},(((((...............))))),,..({({{{{{......................,)))))},........ [-14.61] |
| | ................((((...............))))...................(((((...............)))))....(((............................))))))......... |
| | {-6.30 = -6.30 + -0.00 d = 11.52} |
| 104 | UG(N)xAUGGGAGUUGCCGGUGUAU(N)xUGGGCGCUGC(N)xUUUG(N)xCUAUGCGCUAUUCAUGGU |
| | GCUACC(N)xGUAGAAAAUACUUUAUUU |
| | ...((((.(((.((((.((......................(((((.........................................)))))...))))))))).)).))..................... |
| | (-16.90 = -16.90 + 0.00) |
| | ...((((.(((.((((.((......................(((((.........................................)))))...))))))))).)).))..................... [-18.99] |
| | ...((((.(((.((((.((......................(((((.........................................)))))...))))))))).)).))..................... |
| | {-14.90 = -14.90 + -0.00 d = 8.42} |
| 105 | UUUGAAG(N)xAUGG(N)xUGAUG(N)xGU(N)xGCAAAUACAUUCCGUGCG(N)xUUUAACCCAACCCAA |
| | GCUGAAGAAACUUAUUCAAUGGUCACCGC(N)xUAACCGCUUUUGGUCCCAAAUCUUUGGGGUUGC |
| | U |
| | ....................((..((((.........((..........(((...((..............))....)))..........)).))))))......(((......)))(((((....)))))...... |
| | (-19.20 = -19.20 + 0.00) |
| | ..................,{((..{(((,........{{........,,\|\|\|,}((..............))....)))..........)).))))))......(((......)))(((((....)))))...... |
| | [-22.28] |
| | ....................((..((((.........((..........(((...((..............))....)))..........)).))))))......(((......)))(((((....)))))...... |
| | {-14.84 = -14.84 + -0.00 d = 21.01} |
| 106 | A(N)xUUCGUGCGGCUGAAGA(N)xCCCUGA(N)xAUUUGAGACUUUC(N)xUA(N)xCACCAAAAAUAUUC |
| | UCUUAAACGAAGGUAUUCGUGCUUG |
| | .................(((((((....(((.....(((((((......................)))))))...)))..)))).))).. (-10.90 = -10.90 + 0.00) |
| | .................(((((((....(((.....(((((((......................)))))))...)))..)))).))).. [-11.61] |
| | .................(((((((....(((.....(((((((......................)))))))...)))..)))).))).. {-10.90 = -10.90 + -0.00 |
| | d = 3.80} |
| 107 | UGCUUGGAUGGCGGCUCAAGAUC(N)xAGCCUC(N)xAUGAAAACC(N)xUUAUAUUCCCUGAGGAGGU |
| | UCUACCCCGUGGAAACGCUC |
| | .(((.....)))((((.....................)))).......................................................................................... |
| | ..............((((....((.(((...)))))..))))... (-13.60 = -13.60 + 0.00) |
| | .(((.....)))((((.....................))))........................................................................................., |
| | (........((,..(,,,.,},\|\|,{{({{{{\|,.,}}))}}.,.}.. [-15.35] |
| | .(((.....)))((((.....................))))............................................................................................. |
| | ................(((( ... )))). {-7.90 = -7.90 + -0.00 d = 12.41} |
| 108 | GCAGGGUUUGAAAAAGGAAUUGAUCGUGAUUUUGAACCUGUUCUUUCCAUGACCCCUCUAAAUUG |
| | A(N)xGACAG(N)xGA(N)xAAUC(N)xCAAUACU(N)xUGAAGUAGGAAU(N)xCACU(N)xU(N)xUGA(N)xU( |
| | N)xU(N)xCUAUGAUACAUAU(N)xU(N)xG(N)xA(N)xGA(N)xU(N)xCAGAUCAU(N)xACUU(N)xA(N)xA( |
| | N)xA(N)xAAAGAUUCCUU(N)xAUUU(N)xC(N)xUUUU(N)xUU(N)xU(N)xUUUUC(N)xAA(N)xC(N)xU(N |
| | )xCAUU(N)xUC(N)xAUUUAUAUCUA(N)xAUAUACUUUU |
| | ...(((((.....(((((((......)))....)))))))....)))))............................................................................................ |
| | ........................................................................................................................................... |
| | ...........................(((..((((((.......................................(((((....................................................... |
| | ..........))))).....(((.................))))))))).......)))........................................................................... |
| | ..................................... (-16.00 = -16.00 + 0.00) |
| | ...(((((.,...(((((((......)))....)))))))....))))).....,...................................................................................... |
| | ......................................................................................................................................,... |
| | .,..........................,((\|\|((((((.......................................(((((...................................................... |
| | ...........))))).....((,................})))))))......}},.......................................................................... |
| | ........................................ [-19.45] |
| | ...(((((.....(((((((......)))....)))))))....)))))......................................................................................... |
| | ............................................................................................................................................ |
| | ...................................(((((.......................................(((((........................................................... |
| | ......))))).....((..................))))))))).......................................................................................... |
| | ................................ {-12.40 = -12.40 + -0.00 d=24.47} |
| 109 | (N)xCU(N)xUUGACUGU(N)xAC(N)xAAG(N)xA(N)xAUAU(N)xGUGGAGCUCAGU(N)xAUGUCUGGAA |
| | GCACAGG |
| | ...........................................................................................((((((...((....................................... |
| | ...................))..)).))))............................................................................................................... |
| | ........................................................................................................................................... |
| | ........................................................................................................(((.......))). (-5.50 = -5.50 + 0.00) |
| | ...........................................................................................,{{{((...{{................................ |
| | ..........................}}..}}.\|\|\|\|...................................................................................................... |
| | ..................................................................................................................................... |
| | ........................................................................................................................\|\|\|....,.)}}. [-6.64] |
| | .............................................................................................((((...((................................... |
| | .......................))..)).))....................................................................................................... |
| | ........................................................................................................................................ |
| | ......................................................................................................................(((......))). |
| | {-4.20 = -4.20 + -0.00 d = 9.02} |
| 110 | (N)xAAUAGAUUAG(N)xAUUCUUGUU(N)xAGG(N)xAUUUU(N)xGACACGUGUAGAU(N)xA(N)xUA(N) |
| | xGAAUU(N)xCAA(N)xC(N)xU(N)xGA(N)xA(N)xUUUAUU(N)xGAUCAUUACAU(N)xAGA(N)xA(N)xUU |
| | C(N)xAAUUAAG(N)xAUAU(N)xUGUAUGA(N)x |
| | ............................................................((.....(((((.....)))...............))))......................................... |
| | ........................................((((.(((.......................................)))))))................... (-4.40 = -4.40 + 0.00) |
| | ............................................................,,.....{{{{{,,,,.\|\|}...............}}}}}..}}},.,{,\|,,,,.................... |
| | ...............,,,,,.........................,\|\|\|..,\|\|\|.............}}...},................................................ [-7.35] |
| | ...................................................................((((......))................))......................................... |
| | ................................................................................................................... |
| | {0.30 = 0.30 + -0.00 d = 17.03} |
| 111 | UCAUUACAU(N)xAGA(N)xA(N)xUUC(N)xAAUUAAG(N)xAUAU(N)xUGUAUGA(N)xA(N)xAGU(N)xAU |
| | G(N)xAUUUCUUCUAU(N)xUCUC(N)xUU(N)xUUGAGA(N)xAUUGG(N)xAGGAU(N)xU(N)xUU |
| | ((((.(((............................................)))))))...............................((((((((.((((..........))))............... |
| | ..............)).))..............................))))........ (-14.20 = -14.20 + 0.00) |
| | {{.,{{{.............................................}}}}}}}...............................((((((((.((((..........)))).................. |
| | ...........)},))..............................))))........ [-15.88] |
| | (((..(((............................................))).)))...............................((((((((.((((..........))))................. |
| | ............)).))..............................))))........ {-11.90 = - 11.90 + -0.00 d = 9.38} |
| 112 | CUUCUAU(N)xUCUC(N)xUU(N)xUUGAGA(N)xAUUGG(N)xAGGAU(N)xU(N)xUUUG(N)xAUU(N)xGG |
| | G(N)xUG(N)xGGU(N)xU(N)xCAAAGAAA(N)xAA(N)xGAA(N)xGGGCU(N)xUUU(N)xU(N)xGU(N)xCUA |
| | CCUU(N)xACUU(N)xC(N)x |
| | (((((((.((((........)))).................)).))....................))).....((((.(((............................................. |
| | ....)))......))))..................((((.....)).)).................. (-14.20 = -14.20 + 0.00) |
| | (((((((.((((........)))).................)),))....................))).....,,,,.,,,................................................ |
| | .\|\|}......,}}\|..................(({{.......}}.}}.}}}............ [-16.19] |
| | (((((((.((((........)))).................)).))....................)))............................................................ |
| | .................................((.....)).)).................. {-10.00 = -10.00 + -0.00 d = 11.30} |
| 113 | GG(N)xUG(N)xGGU(N)xU(N)xCAAAGAAA(N)xAA(N)xGAA(N)xGGGCU(N)xUUU(N)xU(N)xGU(N)xCU |
| | ACCUU(N)xACUU(N)xC(N)xAU(N)xUUUUC(N)xCUUAUUUAUA(N)xUCAA(N)xUAAC(N)xUC(N)xAA(N |
| | )xCCAAAAUG(N)xCAAUUAUC(N)xUCCAAGA(N)xACAAAAUG(N)xUCU(N)xGUU(N)x |
| | .............................................((((.........)).))................................................................ |
| | ............................................................................................(-3.00 = -3.00 + 0.00) |
| | ...{{..................({...................,,(({{..........}}.},.}}},......................................{{(({................ |
| | }}.)),...........................................}},{{{.......,.....,.....}}}....................... [-5.78] |
| | ...............................................(((.........)).).............................................{{(({............ |
| | ....)).))............................................(((..................)))....................... {0.20 = 0.20 + -0.00 d = 12.09} |
| 114 | (N)xAAUCUUUAAUAC(N)xU(N)xAUC(N)xC(N)xUA(N)xGAAGAUUCAUG(N)xA(N)xUUU(N)xGU(N)xU |
| | C(N)xGAGAAAAAAAUU(N)xC(N)xUAACAAUUC(N)xAGA(N)xAGAU(N)xCAACUAAGUCUUACAGUAU |
| | GAAC(N)x |
| | ......................................((((((........................................))))))(((((......((.....((.....((((............. |
| | .............................................)))))))))))))........................... (-4.50 = -4.50 + 0.00) |
| | ......................................{(((((........................................)))))}(((((......((.....((.....(({{............ |
| | ..............................................))},)))))))))........................... [-6.17] |
| | ......................................((((((........................................))))))(((((......((.....((.....((................ |
| | .............................................)).)))))))))........................... {-2.04 = -2.04 + -0.00 d = 6.64} |
| 115 | UACCAUUC(N)xCG(N)xUUGUAAAUAGACGAU(N)xCUUA(N)xUCA(N)xUAG(N)xAUC(N)xCAU(N)xUG |
| | GGGUCUUGAAAUUAAAUCAUAGAAU(N)xA(N)xAUGGAA(N)xGCAGCAACCCUACUCGCUAUCUUUA |
| | UAUCUGGUUUACUU |
| | ..............................(((((((((.(((..((.....................)).)))..........((((((..((........((((................................ |
| | ..............................................................))))................))))))))................))).)))))).. (-13.90 = -13.90 + 0.00) |
| | ..............................,((((((({.(((..((.....................)).)))..........((((((.,((........((((................................ |
| | ..............................................................))))................))))))))........,...,..,})))))}}}}.. [-16.10] |
| | ..............................(((((.((..(((..((.....................)).)))..........((((((..((........((((............................... |
| | ...............................................................))))................))))))))................))..)))))).. |
| | {-10.60 = -10.60 + -0.00 d = 13.67} |
| 116 | GUG(N)xAA(N)xU(N)xACCG(N)xCCA(N)xCUAUCUG(N)xUAG(N)xAA(N)xAGA(N)xUAU(N)xCGGGUG |
| | UGAAUC(N)xUACUG(N)xGUCGAUCUAU(N)xCU(N)xAUCCGU(N)xAUAU(N)xAG(N)xAUGUAUGAUC |
| | CCACA(N)xUGC(N)xCCAUU |
| | (((..............((..((........................(((((...............)))..))....))))............................................ |
| | ................(((((((.((............((.........................))....))..)))...)))).)))............. (-16.20 = -16.20 + 0.00) |
| | {{{..............{(..((........................(((((...............)))..))...,)}}},,............................................. |
| | .............((({(((.((............((.........................))....))..)))...)))}.}}),...,,,...... [-17.33] |
| | .................((..((........................(((((...............)))..))....))))........................................... |
| | .................((((((.((.............((.........................))....))..)))...))))................. {-15.30 = -15.30 + -0.00 d = 10.54} |
| 117 | (N)xUACUG(N)xGUCGAUCUAU(N)xCU(N)xAUCCGU(N)xAUAU(N)xAG(N)xAUGUAUGAUCCCACA(N) |
| | xUGC(N)xCCAUUU(N)xG(N)xUUUUGU(N)xGAA(N)xA(N)xAAAAAAUA(N)xAA(N)xA(N)xUU(N)xUCA |
| | UU(N)xCUCCCCCGAUU(N)xCC(N)xAU(N)x |
| | ...............................................((((....((.((.....((...............))....))..))(((((......))...))).... |
| | ....................................(((((........................))).))...........................))))................ (-8.10 = -8.10 + 0.00) |
| | ...............................................((((,,,,((.((.....((...............))....))..))(((((......))...)))................. |
| | .......................{{{{,........................,,...............................))))................ [-9.61] |
| | ...............................................((((....((.((.....((...............))....))..))(((((......))...)))..................... |
| | .................................................................................))))................ {-7.40 = -7.40 + -0.00 d = 8.97} |
| 118 | (N)xAA(N)xA(N)xUU(N)xUCAUU(N)xCUCCCCCGAUU(N)xCC(N)xAU(N)xGU(N)xACGAAU(N)xA(N)xA |
| | A(N)xGUG(N)xGUAAAAAA(N)xGGCAAUAAGAA(N)xGUCAU(N)xAUAG(N)xAAU(N)xCAAUGGAUUCA |
| | UAGUCAUGGUAAAAUC(N)xCCU |
| | ...............................................((.........((.((.................((....................................))............... |
| | (((...........)))..........................))))..))..................... (-4.00 = -4.00 + 0.00) |
| | ...............................................(({,.......((.((.................{{....................................,,...,,.......... |
| | (((.....,,,...}}\|,,........................}}}}.}}}...}}}}}))...,,,,........ [-6.11] |
| | ....................................................................................................................................... |
| | (((.........))).......................................................{-1.50 = -1.50 + -0.00 d = 14.58} |
| 119 | (N)xGUG(N)xGUAAAAAA(N)xGGCAAUAAGAA(N)xGUCAU(N)xAUAG(N)xAAU(N)xCAAUGGAUUCA |
| | UAGUCAUGGUAAAAUC(N)xCCUGUAUGAUU(N)xUA(N)xU(N)xUUUAGUAAAAUU(N)xUUUGACUG |
| | AUAGAGGGAUCA(N)xA(N)xAUGGUAUA(N)xGUUCAUUU(N)xGUU(N)xGGUAGCU |
| | ....................(((.................(((((.((((..((.....((.(((..........))))))).....................)))))))))....................... |
| | .....................(((((((((.((((.................................)))).)))......)))..))).))) (-14.70 = -14.70 + 0.00) |
| | ....................(((.................(((((.((((..((.....((,(((,,,.......))))))).....................)))))))))...................... |
| | ......................(((((((((.((((.................................)))).)))......)))..))).}}, [-17.20] |
| | ....................(((.................(((((.((((..((.....((.(((..........))))))).....................)))))))))..................... |
| | .......................(((((((((.((((.................................)))).)))......)))..))).)). {-10.40 = -10.40 + -0.00 d = 17.72} |
| 120 | UAUA(N)xGUUCAUUU(N)xGUU(N)xGGUAGCU(N)xUGGAGGAUUAAAAGC(N)xAUGACUCUUGCUU |
| | UCCAAUUGGCUGUUUUUGCAUUAAUUG |
| | .(((....................................((.((..............((..((.(((.....((((.................................................... |
| | .................... ...................................................................................)))).)))..))))))))))))..... |
| | .............. (-13.60 = -13.60 + 0.00) |
| | .(((....................................({,((..............((..((.(((.....((((..................................................... |
| | .......................................................................................................)))).)))..))))))))))) |
| | )................... [-14.52] |
| | .(((....................................((.((..............((..((.(((.....((((........................................................ |
| | ...................................................................................................)))).)))..))))))))))))........... |
| | ........ {-13.60 = -13.60 + -0.00 d = 2.98} |
| 121 | GGUACAUCAUUAUGGAUUGGAUUAGUCUUUCUG(N)xGUAGGUAUCCUUAAUUCUCUCAUC(N)xUC |
| | UUGA(N)xACCUAUUCGUCCCAGAUCCA(N)xAA(N)xAC(N)xCGAAAUG(N)xAC(N)xCCCCCC(N)xA |
| | ...........(((((((((((((.....)))...............((((((..................................................................................... |
| | ......................................................................................................................................... |
| | ..............................))))))..)))..))))))........................................................................................... |
| | ................................................................................................................... (-10.90 = -10.90 + 0.00) |
| | ...........(((((((((((((.....)))...............((((((................................................................................. |
| | ........................................................................................................................................ |
| | ...................................))))))..)))..))))))................................................................................ |
| | .............................................................................................................................. [-12.44] |
| | ...........(((((((((((((.....)))...............((((((............................................................................. |
| | ................................................................................................................................... |
| | ............................................))))))..)))..)))))).................................................................... |
| | ........................................................................................................................... |
| | ............... {-10.90 = -10.90 + -0.00 d = 12.20} |
| 122 | (N)xCCCCCC(N)xAACGCUU(N)xCU(N)xCGGUUGU(N)xAAA(N)xACACA(N)xU(N)xUAUAA(N)xUUC(N) |
| | xAAUACA(N)xA(N)xUA(N)xU(N)xA(N)xAG(N)xUUCCAAAA(N)xA(N)xUGCAU(N)xUAUAA(N)xUU(N)x |
| | C(N)xA(N)xAU(N)xACAAUAUAAG(N)xUUCCAAAAA(N)xUGCAA(N)xAUAAAGA(N)xAAA(N)xCAA(N)x |
| | A(N)xAAA(N)xUGAG(N)xAG(N)xGGG(N)xGG |
| | .............................................................................................................................(((( |
| | (.......((.((.......((..(((............)))............................................................................................. |
| | ...................................................................................................................((((........... |
| | .......................................................))))...................................))))..))......)))..)) (-10.87 = -10.87 + 0.00) |
| | .............................................................................................................................(((((... |
| | ....((.((.......({,,(((............},.............................................................................................. |
| | ...................................................................................................................((((............ |
| | .....................................................)))))...................................,)))..))......)))..)) [-13.28] |
| | .......................................................................................................................... |
| | ...(((((.......((.((.......(...................................................................................................... |
| | ................................................))...............)))..))......)))..)) {-8.26 = -8.26 + -0.00 d = 9.00} |
| 123 | A(N)xU(N)xUAUAA(N)xUUC(N)xAAUACA(N)xA(N)xUA(N)xU(N)xA(N)xAG(N)xUUCCAAAA(N)xA(N)x |
| | UGCAU(N)xUAUAA(N)xUU(N)xC(N)xA(N)xAU(N)xACAAUAUAAG(N)xUUCCAAAAA(N)xUGCAA(N)x |
| | AUAAAGA(N)xAAA(N)xCAA(N)xA(N)xAAA(N)xUGAG(N)xAG(N)xGGG(N)xGGU(N)xC(N)xA(N)xA(N)x |
| | ACUU(N)xCU(N)xUGUGCUUGAAUGA(N)xA(N)xAAAAAU(N)xG(N)x |
| | ..................................................................................................................................... |
| | ...................(((...((((((.......................................................((((.........................................)))) |
| | ....................................................((......(((.................)))....)).....)))).)))))............................. |
| | .......................... (-6.37 = -6.37 + 0.00) |
| | ................................................................................................................................ |
| | ........................(((...((((((.......................................................((((......................................... |
| | ))))....................................................((......(((.................)))....)).....)))).))))).................. |
| | ..................................... [-7.62] |
| | ................................................................................................................................... |
| | .....................(((...((((((.......................................................((((.........................................) |
| | )))....................................................((......(((.................)))....)).....)))).)))))...................... |
| | ................................. {-6.37 = -6.37 + -0.00 d = 4.46} |
| 124 | U(N)xAUU(N)xUAGGUAUUUC(N)xAAUUAAAAAACAAAGU(N)xC(N)xA(N)xAAAAAAUAAGAAUU(N)x |
| | GGGUUGCGCCAUAUAUAU(N)xGA(N)xAAGAGUAUACA(N)xAUA(N)xAUG(N)xA(N)xU |
| | ...........................(((.(((.((((.................................))))..))).))).........(((((.............. |
| | )))))......................... (-4.70 = -4.70 + 0.00) |
| | ...........................(((.(((.((((.................................))))..))).))).........(((((......... |
| | .....)))))......................... [-5.77] |
| | ...........................(((.(((.((((.................................))))..))).))).........(((((..............))))) |
| | ......................... {-3.60 = -3.60 + -0.00 d = 6.45} |
| 125 | (N)xAU(N)xU(N)xCUGAU(N)xUAGUUGA(N)xAAA(N)xUAU(N)xU(N)xA(N)xG(N)xUCUUAG(N)xU(N)x |
| | UAGGAA(N)xUUUUUU(N)xG(N)xAAAGAUU(N)xC(N)xCUGU(N)xGAAAAGUU(N)xGC(N)xAUUAACG |
| | CCUAAUU(N)xC(N)xAUGU(N)xCGAGUAGACC(N)xUU(N)x |
| | ........(((..(((........................................................((((......................................... |
| | ..))))((........................((((((.......((.........))...))))))))..((..................))))))))............... (-4.50 = -4.50 + 0.00) |
| | ........(((..(((.,,.....................................................,(((,.......................................... |
| | ))))((....,,,,,,..............((((((.......((.........))...))))))))..((..................))))))))............... [-6.58] |
| | ........(((..(((........................................................(((.............................................))). |
| | (.......................(((((((.......((.........))...))))))))..((..................))))))))............... |
| | {-1.40 = -1.40 + -0.00 d = 13.79} |
| 126 | AGUAGACC(N)xUU(N)xGU(N)xUGUUG(N)xUGAGA(N)xAUUAUUAA(N)xUU(N)xC(N)xAUG(N)xAG(N |
| | )xUUCUAGGGAGGGAUUU(N)xAUGUCACCACAAACAGAGACU(N)xAAAGCAAGUGCUGGAUUCAAAG |
| | CGGGCGUUAAAGAU(N)xUA |
| | ............................................................((..((....(((......(((((........................................................ |
| | ............................................................))).))...........(((...))))))))))....................... (-9.60 = -9.60 + 0.00) |
| | .................,.............,,..........,,((,................,(....((((.....(((((...................................................... |
| | ..............................................................))).)),,,.,((.....,((..,((((,..,),..),)))),),,,......... [-12.54] |
| | ...............................................................................(((((................................................... |
| | .................................................................))).)).................................................. |
| | {-3.40 = -3.40 + -0.00 d = 18.92} |
| 127 | GAGAUCAAAUUUGAA(N)xUUUGAAGCAAUGGAUACUUUGUAA(N)xUC(N)xCAGUAAG(N)xUACC(N) |
| | xG(N)xUUCG(N)xUUCUCUUA(N)xAUUGAAUUGC(N)xAA(N)xUU |
| | ....(((((..................)))).(((((((.(((((.......................................................................................... |
| | ...........................................................................................................................))).))))..... |
| | ...............................((.....................)))))))............. (-7.90 = -7.90 + 0.00) |
| | ....(((((..................)))).(((((((.(((((................................................................................. |
| | ....................................................................................................................................))).)))) |
| | ....................................((.....................)))))))............. [-9.60] |
| | ....(((((..................)))).(((((((.(((((......................................................................................... |
| | ............................................................................................................................))).))))...... |
| | .....................................................)))))))............. {-6.80 = 6.80 + -0.00 d = 9.07} |
| 128 | AA(N)xUC(N)xCAGUAAG(N)xUACC(N)xG(N)xUUCG(N)xUUCUCUUA(N)xAUUGAAUUGC(N)xAA(N)x |
| | UUAAAC(N)xUC(N)xGGC(N)xC(N)xCAAU(N)xCUUUU(N)xACUAA(N)xA(N)xA(N)xGGAU(N)xUGA(N)x |
| | GCCG |
| | ....................................................................................................................................... |
| | ...................................................................(((((((.................................))).........))))............... |
| | ...............................................(((.......((((........((......................)))...))......))). (-11.10 = -11.10 + 0.00) |
| | .................................................................................................................................... |
| | ......................................................................(((((((.................................))).........)))) |
| | ..............................................................(((.......((((........((......................)))...))......))). [-12.07] |
| | ...................................................................................................................................... |
| | ....................................................................(((((((.................................))).........))))................ |
| | ..............................................(((.......((((........((......................)))...))......))). {-8.50 = -8.50 + -0.00 d = 4.78} |
| 129 | CUGGGAG(N)xUUGAGACAGUUC(N)xCAUUUUACUAAAAUACUUUCAUUAGCUCCC(N)xGAGGUCUU |
| | AUAA(N)xAACGA(N)xGACCCUGAUAU(N)xG(N)xGUUA(N)xUAU(N)x |
| | .(((((...((((((.(((....................))).)).))))))))).............((((((.................................................................. |
| | ............................................................................................................................................... |
| | .................................................................................................))....))))................................... |
| | .................. (-21.60 = -21.60 + 0.00) |
| | .(((((...((((((.(((....................))).)).))))))))).............((((((................................................................... |
| | ............................................................................................................................................ |
| | ...................................................................................................))....))))................................ |
| | ..................... [-22.49] |
| | .(((((...((((((.(((....................))).)).))))))))).............((((((................................................................... |
| | ................................................................................................................................................ |
| | ...............................................................................................))....))))................................... |
| | .................. {-21.60 = -21.60 + -0.00 d = 3.38} |
| 130 | AAAAGAAUAAAUAUAGUGAUAAUUUGAUUCUUCGUCGCCGUAGUAAAUAG(N)xG |
| | ..........(((.(((((..............))))).)))......................................................................................... |
| | .......................................................................................................................................... |
| | .......................................................................................................................................... |
| | ........................................................................................................................................... |
| | .......................................................................................................................................... |
| | .......................................................................................................................... |
| | ....................................................................................................... (-3.00 = -3.00 + 0.00) |
| | ...,,,,..(((.(((((....,,)....),)))))).)),..................................................................................................... |
| | ............................................................................................................................................. |
| | ......................................................................................................................................... |
| | ........................................................................................................................................ |
| | .......................................................................................................................................... |
| | ........................................................................................................................................... |
| | ..............................................................................[-4.34] |
| | .........(..(((((....,,)....))))......................................................................................................... |
| | .......................................................................................................................................... |
| | ......................................................................................................................................... |
| | ............................................................................................................................................ |
| | ........................................................................................................................................ |
| | ........................................................................................................................................ |
| | .................................................................................... {0.20 = 0.20 + -0.00 d = 8.27} |
| 131 | CUUG(N)xCAAG(N)xAACG(N)xGAC(N)xUAACAUGG(N)xUCAGCUAC(N)xCCAGC(N)xCAACCUUCAUA |
| | AU(N)xAAAAUACCG(N)xUUACUG(N)xUAUA(N)xGGUAG(N)xAU(N)xCUCA |
| | ......................((....(((...........))).............))...........((((............................))))....... |
| | .................................................................... (-5.50 = -5.50 + 0.00) |
| | .,(..,,......,,((.....((....(((...........))).............))...........,,,(,.........),..),.....)).....)),,......... |
| | .................................................................. [-6.83] |
| | .,....................((....(((...........))).............))....................................................... |
| | ................................................................... {-2.70 = -2.70 + -0.00 d = 8.55} |
| 132 | (N)xCAACCUUCAUAAU(N)xAAAAUACCG(N)xUUACUG(N)xUAUA(N)xGGUAG(N)xAU(N)xCUCA(N)x |
| | UUGUGAAAGACCU(N)xAUUACU(N)xGGAUA(N)xAUUCAU(N)xGGGUAGAGCCAAA(N)xGAAUGUGA |
| | AC(N)xUAUACAAGUU |
| | ............................((((((..........((((................................))))................................. |
| | .........................(((((...((.....................))...........)))))...((.......)).....................))))))........... |
| | (-10.10 = - 10.10 + 0.00) |
| | ............................((((((..........((((................................)))).............................................. |
| | ............,(((,...((.....................))...........)))))...((.......)).....................))))))........... [-12.48] |
| | ............................((((((..........((((................................)))).................................................... |
| | ...............(.....................))...........)))))...((.......)).....................))))))........... {-2.68 = -2.68 + -0.00 d = 14.98} |
| 133 | GUAAAGGCUCCGGUGUA(N)xUAGAGAAGACCUCACCGUUUAAGAA(N)xGUAA(N)xCCAUAGAAACG |
| | AUGGAAUCCACUAUUUCUUUAU |
| | ................(((((....................)))))...(((((........(((......(((......)))).......))))))))... (-10.30 = - 10.30 + 0.00) |
| | ................(((((....................)))))...(((((........(((......(((......)))).......))))))))... [-11.37] |
| | ................(((((....................)))))...(((((........(((......(((......)))).......))))))))... {-9.60 = - 9.60 + -0.00 d = 8.44} |
| 134 | (N)xUUU(N)xU(N)xU(N)xGAUAC(N)xCUA(N)xAUAGAAUACAACUU(N)xCUUAU(N)xUUCG(N)xAA(N) |
| | xGUGAAAUGCC(N)xUAGAAAAAAGAAAAAAUUGU(N)xGGUUGGGAA(N)xG(N)xGUUAUA(N)xGUAG |
| | C(N)xCAA |
| | .............................((((......)).....))(((.(((...........................................))))))((........(((....((........)).. |
| | ..)))))........................(((((...)))))..................... (-5.90 = - 5.90 + 0.00) |
| | .............................((((......)).....))(((.(((...........................................))))))((........(((....((........))... |
| | .)))))........................(((((...)))))..................... [-8.13] |
| | .............................((((......)).....))(((.(((...........................................))))))((........(((....((........)).. |
| | ..)))))........................(((((...)))))..................... {-1.80 = -1.80 + -0.00 d = 14.84} |
| 135 | UA(N)xCAUUGGA(N)xAAAU(N)xAUGUUUUGU(N)xUAUUAAUAGACUAGAAAAGGGACAAAGAAUAA |
| | (N)xC(N)xUGAAAGAA(N)xGGGA(N)xAAUCAAUUCGGUAUUCGUCAAAGUUUCAUUUAUUCA(N)xAU |
| | GACCAGAACUAAACGGG |
| | ..................(((.(((.........((.......)).....((((((.......................(((((.........................(((.......................... |
| | (((.....))).)))..............))))).))))))................................................................................................. |
| | ........................................................................................................................................... |
| | .......................................................................))))).)))))........ (-11.50 = -11.50 + 0.00) |
| | ..................(((((((.........((.......)).....((((((.......................(((((.........................(((................. |
| | .........(((.....))).)))..............))))).))))))........................................................................................... |
| | ............................................................................................................ |
| | ............................................................................................................))))).)))))........ [-13.31] |
| | ..................(((.(((.........((.......)).....((((((.......................(((((.........................(((........... |
| | ...............(((.....))).)))..............))))).))))))........................................................................... |
| | ...............................................................))))..))))) .... ..{-6.20 = -6.20 + -0.00 d = 11.16} |
| 136 | AGUA(N)xGGAGUUAAACUUU(N)xAUGUUCGAGAAGAGACAAACA(N)xGAAGUAUACGCUUUAGGUC |
| | AACAU(N)xAUAGCU(N)xAUGUCUGCGCACAAAGCGCGAAGAGUA(N)xAUUGAUC |
| | ..........((((((((((................................................................................................................ |
| | ............................................................................((.....))(((........(((((....)))))..))).((((.................... |
| | )))).(((((.....))))).)))))........))))). (-13.60 = -13.60 + 0.00) |
| | ..........((((((((((................................................................................................................ |
| | ............................................................................((.....))(((........(((((....)))))..))).((((.................... |
| | )))).(((((.....))))).)))))..)))........))))), [-16.70] |
| | ....................................................................................................................................... |
| | ..........................................................................((.....))(((........(((((....)))))..))).((((.................... |
| | )))).(((((.....))))).)))))......... {-4.00 = -4.00 + -0.00 d = 17.89} |
| 137 | GUGU(N)xGGU(N)xAUAU(N)xUCCUAUCAUAAUAUAGGAACAGUAAGAACUAGC(N)xAUUCU(N)xUA |
| | UUGAG(N)xACU(N)xAGAACU(N)xCAUAGGGA(N)xAUCCAAUAG(N)xAUUU(N)xAUGGAUGGAAUC |
| | ...................................((((((......)))))).(((((((((..................))))...............)))))........................... |
| | ...................................(((((................................................................................................... |
| | ........................................................................................................................................ |
| | ............................................................................)))))....... (-16.20 = -16.20 + 0.00) |
| | ...................................((((((......)))))).(((((((((..................))))...............)))))................................ |
| | ..............................(((((............................................................................................................ |
| | ..................................................................................................................................... |
| | ......................................................................)))))....... [-17.94] |
| | ...................................((((((......)))))).(((((((((..................))))...............)))))................................... |
| | ...........................(((((......................................................................................................... |
| | ..................................................................................................................................... |
| | .........................................................................)))))....... {-16.20 = -16.20 + -0.00 d = 12.50} |
| 138 | U(N)xUAUUGAG(N)xACU(N)xAGAACU(N)xCAUAGGGA(N)xAUCCAAUAG(N)xAUUU(N)xAUGGAUG |
| | GAAUCAAAUAU(N)xGCA(N)xGUAUUUACAGACAAA(N)xAGU |
| | .................((((.....................))...................))....((......((((.((............................................... |
| | .......................................................................................................................................... |
| | ................................................................................................................................ |
| | ))..)))).)).............................................................. (-5.90 = -5.90 + 0.00) |
| | .................((((.....................))...................))....((......((((.((.............................................. |
| | .................................................................................................................................... |
| | ..................................................................................................................... |
| | ..................))..)))).)).............................................................. [-7.71] |
| | .................((((.....................))...................))....((......((((.((.............................................. |
| | .......................................................................................................................................... |
| | ................................................................................................................................ |
| | .))..)))).)).............................................................. {-5.20 = -5.20 + -0.00 d = 10.55} |
| 139 | UAGG(N)xAAAGAAUGAUUGGUUUUG(N)xAACAAUAGA(N)xUGUCUUU(N)xCACAU(N)xCCAA(N)xC |
| | UAAAACA(N)xAUG(N)xAAUAACC(N)xUCUUCAUUUUUAA(N)xAUGGCAGU |
| | ........(((((((((((...............))))(((.....((..((.((.........................))..))..))................. |
| | ))).))))))).................................................................. (-6.80 = -6.80 + 0.00) |
| | ........(((((((((((...............))))(((.....((..((.((.........................))..))..))................. |
| | ))).))))))).................................................................. [-8.45] |
| | ........(((((((((((...............))))(((.....((..((.((.........................))..))..))................ |
| | .))).))))))).................................................................. {-0.51 = -0.51 + -0.00 d = 15.82} |
| 140 | CAUUUUUAA(N)xAUGGCAGUUCCGAAAAAACGU(N)xACUUCUAUA(N)xUCAAAAAAGCGU(N)xAUU( |
| | N)xCGUAAAAAUAUUUGGAAGGGGAAG(N)xGGA |
| | .................................................................................................................((((((....... |
| | ................(((..............)))..........)))))).................. (-6.40 = - 6.40 + 0.00) |
| | .................................................................................................................((((((....... |
| | ................(((..............)))..........)))))).................. [-7.79] |
| | .................................................................................................................((((((.... |
| | ...................(((..............)))..........)))))).................. {-6.40 = -6.40 + -0.00 d = 10.31} |
| 141 | GGG(N)xAAUUCAAAAAGUUUUUUUGUACGACAAACAAAUAAGUCAUAA(N)xAACGUUAGAA(N)xUA |
| | AUCCCAAU(N)xUGAU(N)xUUGA(N)xCUGUCUGAUUUCAUUGUUAAUCUAAAAA(N)xUAAAAUGAAU |
| | AAUUCCCAUU(N)xC(N)xCCUAUUCAUUGGCC(N)xGAA(N)xAACAAUA(N)xGGGAAUGGAAGUAGA(N) |
| | xA(N)xUAAAAAUGC(N)xU(N)xUCUGUU(N)xUACUAAAUUUCUAAAUUAAAAAAAU(N)xAAUUUU(N) |
| | xU(N)xUUG(N)xA(N)xAAAUAAAGAC(N)xAA(N)xGAUACA(N)xAAGUUUU(N)xACC(N)xU(N)xUUC(N)x |
| | UU(N)xUUUAGUAGAUUUU(N)xAUCA(N)xUUU(N)xC(N)xG(N)xAGGGC(N)xGGGG(N)xUCUUA(N)xU |
| | UUUCCC(N)xC(N)xAU(N)xC(N)xA(N)xACC(N)xUAU(N)xU(N)xUAUCUAU(N)xAAUA(N)xAGA(N)xG(N) |
| | xAAA(N)xU(N)xGGG(N)xUUU |
| | (((...................................((((((((((....))))))...(((...(((..................))).............. |
| | .......................................................................................................................... |
| | ........................................................................................................................................ |
| | ..............((((((........................(((.........................................................................................)) |
| | )...........))))))((((...........................................)))))).))))))).........((((((...................))))........................ |
| | ........))))))..................................................................((((......(((..............(((((((.........))))... |
| | .............................((((((..................................................................((((......(((..............)))).. |
| | .......)))))))).........))....)))..................................................................((((......(((..............((. |
| | ..............................(((......)))..............)).............. (-35.80 = - 35.80 + 0.00) |
| | (((...................................((((((((((....))))))...(((...(((..................)))................................................... |
| | .......................................................)))...........))))))((((...........................................)))))).)))))))..... |
| | ....((((((.................................................................................................................................. |
| | .........................................................................................................((((((........................(((.... |
| | .....................................................................................)))...........))))))((((............................... |
| | ............)))))).))))))).........((((((.........(((...................................((((((((((....))))))...(((...(((.......... |
| | ........)))...................................................................)))))))).........))....))).................................. |
| | ................................((((......(((..............((....................... |
| | ........(((......)))..............)).............. [-42.99] |
| | (((.............(....................................................................................................................... |
| | ............................................................................................................................. |
| | ........................................................................................................................... |
| | .................................................................................................................................... |
| | ............((((((........................(((.....................................................))))................................)))))) |
| | ..................................................................((((......(((..............((((((................................ |
| | ...........................................((((((........................(((........................................... |
| | .......)))))))).........))....)))..................................................................((((......(((..............((. |
| | ..............................(((......)))..............)).............. {-24.12 = -24.12 + -0.00 d = 44.10} |
| 142 | UA(N)xAUUGAUUA(N)xUUG(N)xAUAUUGAUU(N)xGC(N)xAGA(N)xGAAAU(N)xC(N)xUUUCA(N)xU( |
| | N)xA(N)xAUUGAAU(N)xU(N)xUCAAGUG(N)xAGU(N)xCAC(N)xUUCUAUUUU(N)xUCC(N)xUU(N)xC |
| | CU(N)xUUCUU(N)xCUUCUUC(N)xGU(N)xUUC |
| | ..........((((........)))).((..(((....((((............))))...........(((((.......))).))................................ |
| | ................................................)))))...)).... (-5.30 = -5.30 + 0.00) |
| | .........{{{,,............}}}},.,,..((.(((.............((((..............))))...................(((((...............))).))... |
| | ..........................................,}}}}...,,.... [-7.22] |
| | .........{{{,,............}}}},.,,..((.(((.............((((..............))))...................(((((...............))).))........ |
| | ......................................)............. {0.80 = 0.80 + -0.00 d = 11.10} |
| 143 | CUAUUUU(N)xUCC(N)xUU(N)xCCU(N)xUUCUU(N)xCUUCUUC(N)xGU(N)xUUCGGAUCGAAA(N)xA |
| | UAGA(N)xA(N)xGCGUU(N)xGA(N)xGUCAAUC(N)xAAA(N)xAA(N)xUCCAA(N)xGGG(N)xAGGUUC(N) |
| | xAUGG |
| | ........................................(((..(((((....(((.......((((...)))).................)))...................((..((............ |
| | ...........))......))).))).............................................................................................................. |
| | ....................))...)).......... (-8.90 = -8.90 + 0.00) |
| | ........................................(((..(((((....(((.......((((...)))).................)))...................((..((................ |
| | .))...))..................))......))).)))............................................................................................... |
| | ................................. [-10.67] |
| | ........................................(((..(((((....(((.......((((...)))).................)))...................((..((............. |
| | ....))...))..................))......))).)))........................................................................................ |
| | ...................................... {-6.20 = -6.20 + -0.00 d = 13.95} |
| 144 | AUUUGUUCCAACCCAAGACAUAAACAACGACAAGGAUAA(N)xUCAUAUUC(N)xGUUAAAAGAC(N)xC |
| | CGA(N)xUGUA(N)xCA(N)xA(N)xAUAAAAC(N)xGAGG |
| | ................(((((....((.......................................................................................................... |
| | .....................................))....))...............)))............................... (-2.50 = -2.50 + 0.00) |
| | ..,(({{....,,....,,{{..,{{....................................................................................................... |
| | .........................................))....))...............))).......................,,,.,,.. [-4.59] |
| | ........................................................................................................................ |
| | ........................................................................................................... {0.00 = 0.00 + -0.00 d = 10.56} |
| 145 | CAAUGAUCU(N)xGAUCAAA(N)xUA(N)xAGAUUA(N)xCU(N)xAUGGUUCAAGAGAAAGUAAGAGUA(N |
| | )xUCUACUCGAACA(N)xCUAGAGUGGAAGUGCGUUGAAUCAAGAGCAGA |
| | ...((((((....................)))))).................................................................................................... |
| | ...................((((((((.........................(((((((.................)))))))......))))))))................ (-17.00 = -17.00 + 0.00) |
| | ...((((((....................))))))............................................................................................ |
| | ...........................{(((((({,..............{{{.......(((((((.................)))))))..))).)))))))}................ [-18.61] |
| | ...((((((....................)))))).................................................................................................. |
| | .....................((((((((...............(((.......(((((((.................)))))))..)))..)))))))................ |
| | {-16.90 = -16.90 + -0.00 d = 8.07} |
| 146 | (N)xCAUUUCGCA(N)xAUUGAUUUACCG(N)xAAAAAUCAGUUUUAA(N)xAU(N)xUC(N)xAUU(N)xCGA |
| | U(N)xUUCUUUCCGCUUUUUUUAG(N)xA(N)xA(N)xAAA(N)xAAGAA(N)xACGG |
| | ................................((((((((...............))))))))............................................................................ |
| | ...................................................................................................................................... |
| | ................................................................................................................................ |
| | ........................................................................................................................... |
| | .......(((.(((((((.................))..)))))...))) (-9.60 = -9.60 + 0.00) |
| | ................................((((((((...............)))))))).......................................................................... |
| | ........................................................................................................................................... |
| | ................................................................................................................................... |
| | ............................................................................................................................ |
| | (((.(((((((.................))..)))))...))) [-10.17] |
| | ................................((((((((...............))))))))............................................................................ |
| | ............................................................................................................................................. |
| | ........................................................................................................................ |
| | ................................................................................................................................... |
| | (((.(((((((.................))..)))))...))) {-9.60 = -9.60 + -0.00 d = 3.12} |
| 147 | U(N)xU(N)xGACAUAAA(N)xAU(N)xGGAUAU(N)xAUC(N)xCAU(N)xAGAUUU(N)xCUGA(N)xC(N)xUC |
| | AUA(N)xUUUAUGAGAUGAUAAA(N)xAU(N)xAUGGCAAAAGUUAUACCGAGAAUUGGUUCA(N)xCGU |
| | (N)x |
| | .................................(((..(((...(((......(((................)))..)))))).)))............................................... |
| | ..........................................................(((((...)))))(((((..)))))....................... (-7.20 = -7.20 + 0.00) |
| | .................................(((..(((...(((......(((................)))..)))))).)))................................................ |
| | .........................................................(((((...)))))(((((..)))))....................... [-9.12] |
| | .................................(((..(((...(((......(((................)))..)))))).)))............................................ |
| | .............................................................(((((...)))))(((((..)))))....................... {-6.60 = -6.60 + -0.00 d = 11.70} |
| 148 | CUUUCAUACGUGAUGUAACCCCUCUGCCACAUAAUGGCUGUAGACCUCCGAAAAAAAGACGUGUGU |
| | AG(N)xAAAUAAACAUUGAA(N)xGAAA(N)xUUUCA(N)xAGAGAAACAAGAGAAAUAAA |
| | ((.(((((((............(((((((.....))))..)))...................))))))).))........................................................... |
| | ..........................................((((........))))................................. (-11.30 = -11.30 + 0.00) |
| | ((.(((((((............(((((((.....))))..)))...................))))))).))........................................................... |
| | ..........................................((((........))))................................. [-13.45] |
| | .......................((((......))))............................................................................................. |
| | ............................................................................................ {-4.40 = -4.40 + -0.00 d = 17.13} |
| 149 | AUA(N)xUUU(N)xC(N)xUAC(N)xAUCUAGGAUC(N)xCGACUUGUAUUA(N)xUGGAUA(N)xC(N)xUAAU |
| | AACUA(N)xA(N)xU(N)xAGGAGCUGAAC(N)xCA(N)xU(N)xU(N)xAUGGCAAGAAAAAGUUUGA |
| | ......................(((.((...........)))))..............((((.............((((..................................................... |
| | ................................))))..,..))))... (-3.20 = -3.20 + 0.00) |
| | ......................(((.((...........)))))..............((((.............((((..................................................... |
| | ...................................................))))..,..))))... [-5.69] |
| | ...........................................................................(((...................................................... |
| | ...................-0.80 = -0.80 + -0.00 d = 11.29} |
| 150 | GUCACAUA(N)xCUUCGUGAAAUGGUUCAUGCAUGUUUGUUGCCAGGGGCGACAAGAUCAAGUUGG |
| | UAA(N)xG(N)xG(N)xA(N)xUU(N)xAAA(N)xAUAU(N)xC(N)xCUUUCAU(N)xUUCA(N)xU(N)xU(N)xUC |
| | UAU(N)xGAUC(N)xGACGAUC(N)xAUAG(N)xAAGAC(N)xCCCUUUACCAUUCUG(N)xUAUAA(N)xA |
| | .((((..............))))((((((..........(((((((((...)))))))))((((...))))........................................................ |
| | .................................................................................................................................... |
| | .......((((.....((((.....................))))......))))........................)))))).............. (-25.00 = -25.00 + 0.00) |
| | .((((..............))))((((((..........(((((((((...)))))))))((((...))))........................................................... |
| | ..................................................................................................................................... |
| | ...((((.....((((.......................))......))))........................)))))).............. [-28.13] |
| | .((((..............))))((((((..........(((((((((...)))))))))((((...)))).............................................................. |
| | ..................................................................................................................................... |
| | ((((.....((((...................))))))......))))........................)))))).............. {-20.00 = -20.00 + -0.00 d = 16.84} |
| 151 | C(N)xCCCUUUACCAUUCUG(N)xUAUAA(N)xA(N)xUGGGCUAUUAUA(N)xUUU(N)xGUA(N)xCAGAU |
| | AU(N)xGGUAGAGG(N)xGGCA(N)xCACUCAAUCCUUGUUUGUCUAU |
| | ............((((((((((((((........(((((......((..........))..)).................))).)))).))......)))))))).................................... |
| | .......................................................................................................((((..((............)).))))... |
| | (-27.00 = -27.00 + 0.00) |
| | ............((((((((((((((........(((((......((..........))..)).................))).)))).))......)))))))).................................. |
| | .........................................................................................................((((..((............)).))))... [-27.54] |
| | ............((((((((((((((........(((((......((..........))..)).................))).)))).))......))))))))..................................... |
| | ......................................................................................................((((..((............)).))))... |
| | {-27.00 = -27.00 + -0.00 d = 1.70} |
| 152 | GCAAG(N)xGCUCAUAACCUUGA(N)xGGUCACGGGUUC(N)xAAAUCCCGU(N)xCUCCGCACUACCCCC |
| | U(N)xUUU(N)xGUCCAAAACUUUAGG(N)xUUUG(N)xACU(N)xCUGGUAACUAG(N)xUA(N)x |
| | .((((............))))....((..(((((........)))))....))....((((.........((.(((........))))............))........))))................. |
| | .....................(-14.40 = -14.40 + 0.00) |
| | .((((............))))....((..(((((........)))))....))....((((.........((.(((........))))............))........)))).................... |
| | .................[-17.20] |
| | ....................((..(((((....)))))....))..........................((.((.(((...)).))...........))................................ |
| | ..................((-9.20 = - 9.20 + -0.00 d = 12.43} |
| 153 | (N)xUUAAUUGAUCAGUUGAAUAUUCGA(N)xAAAUAG(N)xAA(N)xUACGUU(N)xAUA(N)xAA(N)xGA |
| | AUUAAUU(N)xGAUC(N)xAGUUGAAUAUUC(N)xGAGAAACAAAAGUUGAUUG(N)xAG(N)xA |
| | ................................................................((((((((((((((((..(((......((((............................................. |
| | ............................................................................................................................................ |
| | .............................................................................................................................. |
| | ...............................))..............)).........)))))))))......)))).)))))).............((..(((....)))..))...... (-15.50 = -15.50 + 0.00) |
| | ................................................................((((((((((((((((..(((......((((.................................... |
| | ............................................................................................................................................. |
| | ........................................................................................................................................ |
| | .............................))..............)).........)))))))))......)))).)))))).............((..(((....)))..))...... [-17.42] |
| | ................................................................((((((((((((((((..(((......((((.................................. |
| | ................................................................................................................................................ |
| | ................................................................................................................................................ |
| | ....................))..............)).........)))))))))......)))).))))))..................................... {-15.40 = -15.40 + -0.00 d = 6.15} |
| 154 | UUUC(N)xUCAACAUUUAUAUUGA(N)xCA(N)xACAGC(N)xAU(N)xAUCGAACAAAUA(N)xUAAUUC(N) |
| | xAUUG(N)xUGAUAA(N)xGCGG(N)xAUUU(N)xUUUGA(N)xUUUACG(N)xUCCCAUG(N)xGGU(N)xUU |
| | G(N)xGUUUUU(N)xAUC(N)xUUA(N)xC(N)xUUCAUUCAAAU(N)x |
| | ..((..(((...(((((((..................................)))).......))).......)))......)).((..(((..(((.....((.........))...)))..))) |
| | ......................)).................................................... (-4.00 = -4.00 + 0.00) |
| | ..((..(((...(((((((..................................)))).......))).......)))......)).((..(((..(((.....((.........))...)))..))) |
| | .......................).................................................... [-6.54] |
| | ............(((((((..................................)))).......))................................................. |
| | ........................................................................................ {1.80 = 1.80 + -0.00 d = 17.41} |
| 155 | UCUUUUCUAAAAUUCAAAUUCAUUUUAGAUUCGACCGAAAU(N)xAGG(N)xA(N)xUU(N)xUU(N)xGG |
| | GGAU(N)xAAGG(N)xAA(N)xUAAA(N)xC(N)xUAAACAAA(N)xCC(N)xGUGGCUAAAUCCAAGCAACCCU |
| | UU(N)x |
| | .....((((((((........)))))))).....((((((............))......)).)).................................................................... |
| | ........................................................................................................................................... |
| | ............................................................................................................................................. |
| | ............................................................................................................................................. |
| | ...................................................................................................................................... |
| | ..........................................................((.(((.......))).)).......................................................... |
| | ...................................................................................(-10.40 = -10.40 + 0.00) |
| | .....((((((((........)))))))).....((((((............))......)).))................................................................ |
| | ........................................................................................................................................ |
| | ........................................................................................................................................ |
| | ............................................................................................................................................. |
| | ............................................................................................................................................ |
| | ................................................................((.(((.......))).))................................................... |
| | ............................................................................................ [-12.57] |
| | .....((((((((........)))))))).....((((((............))......)).))........................................................................ |
| | .................................................................................................................................... |
| | .................................................................................................................................... |
| | ..............................................(((.......))).......................................................................... |
| | ......................................................................{-9.70 = -9.70 + -0.00 d = 8.66} |
| 156 | GGAU(N)xAAGG(N)xAA(N)xUAAA(N)xC(N)xUAAACAAA(N)xCC(N)xGUGGCUAAAUCCAAGCAACCCU |
| | UU(N)xCUGAAAUCCAAGCAAUCUUUUCGUAGGCGUUUG(N)xCCCCCAA |
| | ((((...........((..................................................................................................................... |
| | .......................................................................................................................................... |
| | .............................................................................................................................................. |
| | ............................................................................................................................................ |
| | .....................................................................................................................))..)))............... |
| | ........................................................................................................................................ |
| | ........................(((((..(((......))).))))).................. (-5.10 = -5.10 + 0.00) |
| | {(.............({.......................................................................................................................... |
| | .............................................................................................................................................. |
| | .......................................................................................................................................... |
| | .............................................................................................................................................. |
| | ..............................................................................................................{{.(({ ....... })).,,........... |
| | ............................................................................................................................................. |
| | ................(((((..,((......))).)))))........ ,,.. [-7.39] |
| | .................................................................................................................................... |
| | ............................................................................................................................................. |
| | ............................................................................................................................... |
| | ............................................................................................................................................... |
| | ................................................................................................................................(((......))). |
| | ............................................................................................................................................ |
| | ..................(((((...((......))..)))))............................ {-3.20 = -3.20 + -0.00 d = 10.35} |
| 157 | A(N)xAAUUCCA(N)xA(N)xU(N)xCCGAACUCAA(N)xACG(N)xCAAAU(N)xUGAGGUU(N)xU(N)xUG(N)x |
| | CU(N)xC(N)xGAA(N)xAAA(N)xUCCGAG(N)xAAU(N)xC(N)xUAGAU(N)xUUGA(N)xU(N)xU(N)xA(N)xU |
| | C(N)xGU(N)xGUCG(N)xU(N)xAA(N)xGA(N)xAAAA |
| | .............................................((((..............))))............................................................. |
| | ............................................................................. (-2.30 = -2.30 + 0.00) |
| | .............................................((((..............))))................................................................. |
| | .................................................))...................... [-3.66] |
| | .............................................((((..............))))..................................................................... |
| | ..................................................................... {-2.30 = -2.30 + -0.00 d = 4.17} |
| 158 | UCCAAGGA(N)xCACGCA(N)xAAAAGC(N)xGAUAAUAGAUCGCGUCGUUAA(N)xGA(N)xAAUGUUAAU |
| | AAAAAU(N)x |
| | ..............................((................(((.....)))))...(((((............................................................... |
| | ...........................................................................................................)))))........ (-6.00 = -6.00 + 0.00) |
| | ..............................((................(((.....)))))...(((((............................................................... |
| | ...........................................................................................................)))))........ [-6.83] |
| | ..............................((................(((.....)))))...(((((.................................................................... |
| | ........................................................................................................... |
| | ........ {-5.50 = -5.50 + -0.00 d = 4.12} |
| 159 | U(N)xUUAUUUUUAUUU(N)xCAGAGUA(N)xC(N)xCUUG(N)xUCUCU(N)xUUU(N)xUUUAAC(N)xA(N) |
| | xAGG(N)xGGG(N)xGGAG(N)xAA(N)xAUGACCAGAAGGUAUUGGAACAUCAAUUUGGAAGA |
| | ...................(((..((((..........(((..........(((((...............)).)))................)))........................................ |
| | ................................................................................................................................... |
| | ....................................................................................................................... |
| | ...............................................(((....))).(((.......))))))))).. (-10.20 = -10.20 + 0.00) |
| | ..............(((..((((...............(((............(((((,(,(..,,........,)).))).......,)))......................................... |
| | ....................................................................................................................................... |
| | ............................................................................................................ |
| | ............................................................(((,...))).(((,....,}},))))))}.. [-12.64] |
| | ..............(((..((((...............(((............(((((,(,(..,,........,)).))).......,))).......................................... |
| | ........................................................................................................................................ |
| | ....................................................................................................................................... |
| | ...............................(((....)))..........)))))).. {-7.50 = - 7.50 + -0.00 d = 17.49} |
| 160 | UCAAUCCGAUUAAUUCUUAACAAAUUAGUAUUCGCAAUUUGUGAGGGCCGUUCUAGCUAU(N)xAU |
| | AAGAAAUCCGUAA(N)xUUA(N)xAU(N)xAAU(N)xAA(N)xGAA |
| | .......((((...((((((((((((.((...)))))))))...(((......)))..................)))))))))................................................. |
| | .................................................................................................................................. |
| | ............................... (-7.00 = -7.00 + 0.00) |
| | .......((((...((((((((((((.((...)))))))))...(((......)))..................)))))))))................................................. |
| | ................................................................................................................................ |
| | .................................. [-9.59] |
| | ...................(((((((.((...))))))))............................................................................................. |
| | ................................................................................................................................. |
| | ............................... {-2.50 = -2.50 + -0.00 d = 14.62} |
| 161 | CUAU(N)xAUAAGAAAUCCGUAA(N)xUUA(N)xAU(N)xAAU(N)xAA(N)xGAAAAAUAACUUCUUUUGG |
| | AA(N)xACUUCA(N)xUAGA(N)xU(N)xUU(N)xAU(N)xGGAAUC(N)xCG(N)xC(N)x |
| | ......................(((((((................................................................................................... |
| | ......................................................................................((...(((...........))))).((((.... |
| | ))))..........................)).))...))).................................... (-4.80 = -4.80 + 0.00) |
| | ......................(((((((............................................................................................... |
| | ..........................................................................................((...(((...........))))).((((.... |
| | )))).......................... ....................... [-6.56] |
| | ......................(((((((................................................................................................... |
| | ......................................................................................((...(((...........))))).((((....)))).......... |
| | ....................................... {-5.10 = -5.10 + -0.00 d = 5.90} |
| 162 | UAGGCAUCAAAAUUUGGAUAUUUAUAGAGGGGGAAUAA(N)xUAAAGCU(N)xUUACUUG(N)xU(N)x |
| | UU(N)xUU(N)xCU(N)xG(N)xUCUU(N)xA(N)xU(N)xUG(N)xAGU(N)xG(N)xAUGA(N)xAA(N)xCAAAAA |
| | A |
| | ...........................(((((..((((.((((....................................................................................... |
| | ................................................................................................................................ |
| | ....................................))...))....))))......))....)))...................... (-7.70 = -7.70 + 0.00) |
| | ...........................(((((..((((.((((....................................................................................... |
| | ....................................................................................................................................... |
| | .............................))...))....))))......))....)))...................... [-8.91] |
| | ...........................(((((..((((.((((............................................................................................ |
| | ................................................................................................................................ |
| | ...............................))...))....))))......))....)))...................... {-7.70 = -7.70 + -0.00 d = 7.05} |
| 163 | U(N)xAUAU(N)xCAAAA(N)xUUUUGA(N)xG(N)xGCAAAAAAAAAU(N)xUGA(N)xAU(N)xUCAUC(N)xA |
| | UGGGUAGAGAUACUAUUGCUGACAUAAUAACCUCUAUACGAAAUGCUGAUAU |
| | ...((((..............((...(((((....................................................((((.................................. |
| | ................................................................................................................))))((((((....))))).. |
| | )))))).)))))...)))))) (-7.50 = -7.50 + 0.00) |
| | ...((((..............((...(((((....................................................((((................................................... |
| | ...............................................................................................))))((((((....)))))..)))))).)))) |
| | )...)))))) [-9.76] |
| | ...((((..............((............................................................((((............................................... |
| | ....................((((............................................................................(((()....((((.....)))..))))...... |
| | )))))) {-3.47 = -3.47 + -0.00 d = 11.12} |
| 164 | UUGAACAAAAAGCUGCUGAAUUGGCCUCUUUCUUGCGUGUACCAAUUGAAGUAUUUUGA(N)xGAA |
| | AUG(N)xGGCUGAAA(N)xAUGAACGCUU(N)xUCUUAGCGGGA(N)xGGGAA(N)xA(N)xA(N)xAAUUC(N |
| | )xA(N)xA(N)xAAAUCCU(N)xC(N)xUUUC(N)xUUUUUU(N)xCUAUA(N)xA(N)xC |
| | ...............................(((((....(((((((((((((..((.((..(((..((................................................................ |
| | ...................))...)).))))..........................)))))).......))).........))))))................................................ |
| | ....................... (-17.40 = -17.40 + 0.00) |
| | ...............................(((((....(((((((((((((..((.((..(((..((.................................................................... |
| | .......................................................................................................................................... |
| | .................))....)))..))...)).))))..........................)))))).......))).........))))))..................................... |
| | .................................. [-18.65] |
| | ...............................(((((....(((((((((((((..((.((..(((..((.................................................................... |
| | .............................................................................................................................................. |
| | .............))....)))..))...)).))))..........................)))))).......))).........))))))..................................... |
| | .................................. {-16.30 =-16.30 + -0.00 d = 11.57} |
| 165 | UA(N)xAUU(N)xCUAAUC(N)xA(N)xUUU(N)xUAU(N)xU(N)xUA(N)xUACGACUUC(N)xUCUUA(N)xAU |
| | (N)xAC(N)xA(N)xAAA(N)xUAAG(N)xAU(N)xAGGAAA(N)xAGUA(N)xA(N)xCAAUCUU(N)xCA(N)xC(N) |
| | xCUUUAUAUGUAAAA(N)xCUC(N)xU(N)x |
| | ..........................................(((..(((...((((............................))))...........)))............)))............... |
| | .......................................................................................................................... |
| | ........................ (-1.90 = -1.90 + 0.00) |
| | ..........................................(((..(((...((((............................))))...........)))............))).............. |
| | .......................................................................................................................... |
| | ......................... [-3.12] |
| | .....................................................((((............................))))............................................ |
| | ................................................................................................................................... |
| | ............... {-0.60 = -0.60 + -0.00 d = 8.77} |
| 166 | CUUG(((((UUCAUAGAAACAAAUGA(N)xACG(N)xU(N)xU(N)xUAGUU(N)xUUGUUCC(N)xU(N)xUGUAAA( |
| | N)xAAAA |
| | .(((((......)))))..................................................................................................................... |
| | ......................................................................................................................................... |
| | .................................................................................................................................. |
| | ........................................................................................................................................ |
| | ...................................................................................................................................... |
| | ............................................................................................................................................... |
| | ...................................................................................................(-1.40 = -1.40 + 0.00) |
| | .,{{{{,....}}\|\|\|.............................................................................................................. |
| | ..................................................................................................................................... |
| | ............................................................................................................................................... |
| | .......................................................................................................................................... |
| | ........................................................................................................................................... |
| | ....................................................................................................................................... |
| | ..............................................................................,,,.................. [-2.55] |
| | ........................................................................................................................ |
| | ....................................................................................................................... |
| | ................................................................................................................................................. |
| | ................................................................................................................................................. |
| | ................................................................................................................................................. |
| | ...................................................................................................................................... |
| | ............. ............................................................................................ {0.00 = 0.00 + -0.00 d = 6.19} |

Examples of four consensus structures can be seen in FIGS. 10A-D. FIG. 10A is the consensus structure (structure #102) that appears in the gene psbC in the 5′ UTR, its product is photosystem II CP43 chlorophyll apoprotein. The start position of the structure is nucleotide 806 upstream of the start codon, its length is 75 nt, its local folding energy is-11.9 kcal/mol, its frequency is 0.9994 (which mean that 99% of the sequence's copies have the specific structure, which is considered very high), and its diversity is 12.65 (which means that there are 12.65 different structures present in the sequence's copies).

FIG. 10B shows the consensus structure (structure #42) that appears in the gene infA in the 5′ UTR, its product is translation initiation factor IF-1. The start position of the structure is at nucleotide 244 upstream of the start codon, its length is 288 nt, its local folding energy is-18.6 kcal/mol, the frequency is 0.9988 (very high), and the diversity is 20.5.

FIG. 10C shows the consensus structure (structure #5) that appears in the gene atpB in the 3′ UTR, the product of this gene is ATP synthase CFI subunit beta. The start position of the structure is at nucleotide 82 upstream of the stop codon, its length is 85 nt, its local folding energy is-10.9 kcal/mol, the frequency is 0.9992 (very high), and the diversity is 24.47.

FIG. 10D shows the consensus structure (structure 133) that appears in the gene rp120 in the 5′ UTR, the product of this gene is the ribosomal protein L20. The start position of the structure is at nucleotide 880 upstream of the start codon, its length is 94 nt, its local folding energy is-10.3 kcal/mol, the frequency is 0.9996 (very high), and the diversity is 13.19.

There is no generic large-scale computational model of translation initiation in chloroplasts that can be used for all genes and all organisms. Therefore, the general aim of this study was to develop novel quantitative models that connect mRNA translation to mRNA and rRNA local folding in chloroplasts; these models help in understanding the evolution and biophysics of chloroplasts. The translation mechanism in the chloroplasts genome was studied by conducting an energy-based model (ETIP) that efficiently predicts protein levels in different ortholog orthologous groups that are composed of chloroplasts genes. Based on the ETIP, the local folding of the mRNA and the local interaction between the mRNA and the 16S rRNA of the small ribosomal subunit were studied. In addition, functional secondary structures that have a wide consensus in genes that belong to the same ortholog group of different chloroplasts genomes were inferred. The models were validated via the analysis of PA values and ribosomal profiling values of Chlamydomonas reinhardtii's genes. This is a green unicellular alga that is widely used as a model system for studying fundamental aspects of chloroplasts and is also widely used as a model in biotechnology. These results demonstrate the biotechnological promise of the models. In addition, for the first time, there was created a database of 77 orthologous groups out of 4,300 different chloroplasts genomes.

The predictive energy-based model of translation efficiency is based on the local folding of the mRNA and the local mRNA-rRNA hybridization; it has different parameters for different orthologous groups. Based on this analysis, for each ortholog group there was inferred the typical local energy parameters that are expected to fit to translation initiation regulation. The optimized correlation across all genomes between the energy model and the Z-scored CAI values is r=−0.63 with Pv<10\hat{ }−324.

The model also efficiently predicts the Z-scored CAI values when a correlation is computed for each genome separately (median correlation of r=−0.64 and Pv=6×10\hat{ }−10 for the genome with the median correlation) which supports the conjecture that this model is generic and universal for all genes in all chloroplasts. Moreover, CAI scores are known to be highly correlated with PA values and it was shown that adding the energy-based model to the CAI scorescan further improved the correlation with PA values. In all cases comparisons to the null model were provided that support the conjecture that the results are meaningful.

The model is composed out of four variables, that describe the local structure, and a parameter (the C constant) whose purpose is to deal with and correct second-order aspects of translation regulation that are not directly considered in the model. Although it has been well analyzed and shown that PPR proteins support translation by interacting with the 5′ UTR of some chloroplasts' UTRs, with the current available data it is impossible to specifically add one (or more) proteins (such as PPR) to our models due to the following reasons: 1) First, there is no quantitative data that measure how PPR proteins interact with the mRNA. Note that these interactions are gene and organisms specific. Without such data we cannot infer parameters of a relevant model. 2) The aim of the model we developed here is to enable a generic predictive power but without getting into specific details of the many factors involve in translation (since we do not have data related to all of them). Thus, all these aspects are inferred indirectly via the C constant. 3) There are many additional factors (not only PPR) related to translation and considering only it will yield a non-generic model with nonoptimal performances.

The parameters related to the ETIP vary among the different orthologous groups, suggesting that indeed different gene families in chloroplasts use different translation initiation mechanisms. Recently it was discovered, that in some chloroplasts, translation initiation regulation of some genes is based on SD interaction while for other genes it is not. This model suggests that the typical translation regulation in chloroplasts is not SD dependent; however, there are some gene families that still rely on this interaction. The analysis herein shows that 64% of gene families have the typical translation initiation mechanism with the typical optimized parameters of mRNA window size of 85 nt that starts at the position of 26 nt upstream of the start codon with a 16S rRNA window size of 22 nt from its 3′ end, and with an ETIP constant of 7.

In accordance with prior studies, genes that were found not to be dependent on the SD (e.g., petD, atpB, atpE, rps4, rps7, rbcL, rpl2 and rpls16) were found in the typical groups while genes that require the aSD-SD interaction during translation initiation (e.g., psbA, psbD, psbC, atpH and rps14) were found in the non-typical groups. Furthermore, it was suggested that highly expressed genes in Chlamydomonas reinhardtii rely on SD interaction in their translation initiation regulation. It was shown that genes belonging to the non-typical groups are significantly highly expressed in comparison to the rest of the genes (Pv=0.01); in addition, it was shown that SD interaction at Chlamydomonas reinhardtii's genes in the non-typical groups seems to be stronger than other genes and to appear at the typical positions according to prokaryotes (i.e., positions of 16-8 nt upstream of the start codon). This is not the case for the typical groups, which are lowly expressed compared to other genes, their aSD-SD interaction tends to be weaker than other genes and it appears at positions 35-30 nt upstream of the start codon). Thus, this study supports the conjecture that the typical translation regulation in chloroplasts tends to not rely on a SD motif while some genes families still use it for translation.

Herein is also created a database containing 166 predicted functional mRNA structures that are specific to different orthologous groups in chloroplasts. Mutation of these structures allows for the tuning of translation in chloroplasts.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of predicting translation initiation efficiency of an mRNA comprising a 5′ untranslated region (UTR) and a coding region in a chloroplast, the method comprising:
a. calculating a free folding energy for a region of said 5′ UTR to produce a target free folding energy;
b. calculating a free folding energy for a region of a16S rRNA of said chloroplast to produce an rRNA free folding energy; and
c. calculating a free folding energy for said 5′ UTR region hybridized to said 16S rRNA region to produce a combined free folding energy, wherein a lower combined free folding energy as compared to a sum of said target free folding energy and said rRNA free folding energy indicates translation initiation and the magnitude by which said combined free folding energy is lower is proportionate to translation initiation efficiency;
thereby predicting translation initiation efficiency.

2. The method of claim 1, comprising performing steps a-c for a plurality of regions within said 5′ UTR and selecting the region with the lowest combined free folding energy.

3. The method of claim 1, for predicting protein expression from said mRNA in said chloroplast, wherein the predicted translation initiation efficiency is proportional to said predicted protein expression.

4. The method of claim 1, further comprising confirming said prediction by comparing said predicted translation initiation efficiency to received expression levels of said mRNA in said chloroplast.

5. The method of claim 1, wherein said method is a method of predicting expression level of a protein encoded by said mRNA in said chloroplast and wherein the greater the difference between said combined free folding energy and said sum of said target free folding energy and said rRNA free folding energy the greater the expression level of said protein in said chloroplast.

6. The method of claim 5, further comprising receiving a measure of protein expression levels in said chloroplast of a protein translated from said mRNA and correlating predicted expression levels to said measure.

7. The method of claim 4, wherein said received expression levels are approximated by the codon adaptation index (CAI) in said mRNA.

8. The method of claim 6, further comprising optimizing said correlation, wherein said optimizing comprises providing a plurality of mRNAs of proteins expressed in said chloroplast, selecting a subgroup of said plurality as a training set and a subgroup of said plurality as a test set, selecting a parameter that optimizes correlation between said predicted expression levels in said training set to said measure of protein expression and validating said parameter in said test set.

9. The method of claim 8, wherein said parameter is selected from 5′ UTR region length, 5′ UTR region start position, 16S rRNA region length and a correction factor applied to said sum of said target free folding energy and said rRNA free folding energy.

10. A method of determining a region regulating translation or secondary mRNA structures regulating translation in an mRNA comprising a 5′ UTR, a coding region and a 3′ UTR in a chloroplast, the method comprising:
a. providing a database of sequences of said mRNA in chloroplast in a plurality of species;
b. aligning said sequences based on sequence similarity to produce a multisequence alignment (MSA);
c. calculating a free folding energy for a region of said mRNA and for each sequence aligned with said region in said MSA to produce a target free folding energy;
d. selecting a region in which said target free folding energy is lower than a free folding energy in a null model;
e. performing a method of claim 1 to predict translation initiation efficiency for said selected region of step (d); and
f. selecting a region as a region initiating translation and/or a region whose secondary mRNA structure regulates translation if said combined free folding energy is lower than a sum of said target free folding energy and said rRNA free folding energy and a region as a region terminating translation is said combined free folding energy is higher than said sum;
thereby determining a region regulating translation.

11. (canceled)

12. The method of claim 10, wherein said database comprises sequences from at least 10 different species.

13. The method of claim 1, wherein said region is a window of 25-50 nucleotides.

14. The method of claim 10, wherein said region is within said 5′ untranslated region (UTR) and said regulating translation is initiating translation or said region is within said 3′ UTR and said regulating translation is terminating translation, said method comprises evaluating all possible regions within said mRNA or both.

15. (canceled)

16. The method of claim 1, comprising evaluating all possible regions within said mRNA or both and combining any adjacent regions that all initiated translation or terminate translation to produce a complete initiating region, a complete terminating region or both.

17. The method of claim 1, wherein at least one of:
a. said lower is lower by more than a predetermined threshold, said higher is higher by more than a predetermined threshold or both;
b. said calculating free folding energy comprises calculating relative free folding energy and comprises calculating free folding energy for a null model of said sequence of said region or aligned sequence, wherein said relative free folding energy is the difference between the free folding energy of said region or aligned sequence and said null model; and
c. said region does not comprise a Shine Dalgarno sequence or comprises a Shine Dalgarno sequence at a location that is not between position −1 and −16 with respect to the translational start site of said mRNA.

18. (canceled)

19. (canceled)

20. The method of claim 2, wherein at least one of:
a. said selecting further comprises selecting a region or combined region with relative free folding energy that is below a predetermined threshold, thus selecting a region with a conserved structure;
b. said selecting further comprises selecting a region or combined region with relative free folding energy that is below a predetermined threshold, thus selecting a region with a conserved structure in all species of said plurality of species; and
c. said selecting comprises selecting a region or combined region comprising a relative free folding energy that is significantly lower than the relative free folding energy of both the adjacent upstream and downstream regions.

21. (canceled)

22. (canceled)

23. A method of modulating translation of a target mRNA in a chloroplast, the method comprising determining a region regulating translation in said chloroplast by a method of claim 10; and
i. generating said determined region in said target mRNA which is not an mRNA that naturally comprises said determined region; or
ii. abolishing said determined region in said target mRNA which is an mRNA that naturally comprises said determined region;
thereby modulating translation of a target mRNA.

24. A method of modulating translation of a target mRNA in a chloroplast, the method comprising generating in said target mRNA a region folding into a secondary structure selected from those provided in Table 3 or abolishing in said target mRNA a secondary structure selected from those provided in Table 3; thereby modulating translation of a target mRNA.

25. The method of claim 23, wherein:
a. said generating comprises insertion of said determined region or a region folding into said secondary structure into said mRNA or mutation of a region of said target mRNA to produce said determined region;
b. said generating comprises insertion of said determined region or a region folding into said secondary structure into a DNA encoding said target mRNA or mutation of a region of a DNA encoding said target mRNA to produce said determined region;
c. said abolishing comprises deleting said determined region or region folding into said secondary structure;
d. said abolishing comprises mutating said determined region or region folding into said secondary structure; or
e. said abolishing comprising mutating said determined region or region folding into said secondary structure, wherein said mutating changes the local folding energy of said determined region in said mRNA by at least a predetermined threshold.

26. (canceled)

27. (canceled)

28. The method of claim 23, wherein said generated is at a location in said target mRNA that corresponds to the location of said determined region or region folding into said secondary structure in the mRNA from which it was determined; optionally wherein said determined region or region folding into said secondary structure is located in its original mRNA in a 5′ UTR and is generated in a 5′ UTR of said target mRNA or is located in its original mRNA in a 3′ UTR and is generated in a 3′ UTR of said target mRNA.

29. (canceled)

30. (canceled)

31. (canceled)
